(19) United States
(12) Patent Application Publication
(10) Pub. No.: US 2024/0279171 A1
(43) Pub. Date: Aug. 22, 2024

(54) METHODS FOR PREPARING CRYSTALLINE FORMS OF AMISULPRIDE

(71) Applicant: SUNOVION PHARMACEUTICALS INC., Marlborough, MA (US)

(72) Inventors: Robert Joseph PRYTKO, Marlborough, MA (US); John R. SNOONIAN, Malborough, MA (US); Harold Scott WILKINSON, Malborough, MA (US); Anna BALANOV, Marlborough, MA (US)

(21) Appl. No.: 18/565,618

(22) PCT Filed: Jun. 2, 2022
(86) PCT No.: PCT/US22/31891

§ 371 Date: Nov. 30, 2023

Related U.S. Application Data
(60) Provisional application No. 63/196,591, filed on Jun. 3, 2021.

(51) Int. Cl. C07D 207/09; A61K 31/40

(52) U.S. Cl. CPC C07D 207/09; A61K 31/40

(57) ABSTRACT

Provided are methods for making crystalline forms of (S)-(−)-amisulpride and (R)-(+)-amisulpride. Also provided are pharmaceutical compositions comprising the crystalline forms of (S)-(−)-amisulpride and (R)-(+)-amisulpride and methods of using the crystalline forms.

### FIELD OF THE INVENTION

The present disclosure relates to methods for preparing crystalline forms of enantiomerically pure amisulpride.

### BACKGROUND

Amisulpride is a member of the chemical class benzamide, and has the chemical name 4-amino-N-[(1-ethylpyrrolidin-2-yl)methyl]-5-ethylsulfonyl-2-methoxybenzamide. The chemical structure of amisulpride is as follows:

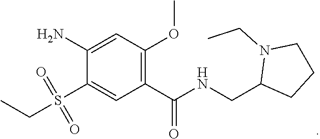 [SMILES: CCN1CCCC1CNC(=O)c1cc(S(=O)(=O)CC)c(N)cc1OC]

Drug substances are most frequently administered orally by means of solid dosage forms such as tablets. Tablets remain popular because of the advantages afforded both to the manufacturer (e.g., simplicity and economy of preparation, stability and convenience in packaging, shipping and dispensing) and to the patient (e.g., accuracy of dosage, compactness, portability, and ease of administration). The preparation of tablets typically requires that the active pharmaceutical ingredient (API) be a solid. In the manufacture of solid APIs, it is necessary to obtain products with reproducible properties, including chemical purity and composition. For crystalline solid enantiomeric APIs, it is important to produce the desired crystalline solid with high chemical and enantiomeric purity. There is a continued need for reliable, reproducible process for preparing pure crystalline amisulpride enantiomers.

Amisulpride has a single asymmetric center and as a result exists in two enantiomeric forms: (R)-4-Amino-N-[(1-ethyl-2-pyrrolidinyl)methyl]-5-(ethylsulfonyl)-2-methoxybenzamide (also referred to as: (R)-(+)-4-amino-N-[(1-ethylpyrrolidin-2-yl)methyl]-5-(ethylsulfonyl)-2-methoxybenzamide, and under the IUPAC name as 4-amino-5-(ethanesulfonyl)-N-{[(2R)-1-ethylpyrrolidin-2-yl]methyl}-2-methoxybenzamide), abbreviated herein as (R)-(+)-amisulpride or (R)-amisulpride; and (S)-4-Amino-N-[(i-ethyl-2-pyrrolidinyl)methyl]-5-(ethylsulfonyl)-2-methoxybenzamide (also referred to as: (S)-(−)-4-amino-N-[(1-ethylpyrrolidin-2-yl)methyl]-5-(ethylsulfonyl)-2-methoxybenzamide, and tinder the IUPAC name as 4-amino-5-(ethanesulfonyl)-N-{[(2S)-1-ethylpyrrolidin-2-yl]methyl}-2-methoxybenzamide), abbreviated herein as (S)-(−)-amisulpride or (S)-amisulpride. These two enantiomeric forms have the following chemical structures:

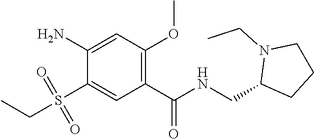 [SMILES: CCN1CCC[C@@H]1CNC(=O)c1cc(S(=O)(=O)CC)c(N)cc1OC]

### (R)-4-Amino-N-[(1-ethyl-2-pyrrolidinyl)methyl]-5-(ethyl sulfonyl)-2-methoxybenzamide (R)-amisulpride

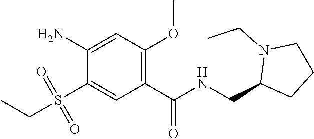 [SMILES: CCN1CCC[C@H]1CNC(=O)c1cc(S(=O)(=O)CC)c(N)cc1OC]

### (S)-4-Amino-N-[(l-ethyl-2-pyrrolidinyl)methyl]-5-(ethylsulfonyl)-2-methoxybenzamide (S)-amisulpride

### SUMMARY

Provided herein is a method of preparing an enantiomerically pure crystalline form of (R)-(+)-amisulpride comprising the steps of:
(a) coupling, in the presence of a tertiary amine and an acid activating reagent, 4-amino-5-(ethylsulfonyl)-2-methoxybenzoic acid and (R)-(1-ethylpyrrolidin-2-yl) methanamine to form a reaction mixture; and
(b) isolating from the reaction mixture of step (a), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°; wherein step (b) does not comprise formation of a solvate of (R)-(+)-amisulpride.

Also provided herein is a method of preparing an enantiomerically pure crystalline form of (S)-(−)-amisulpride comprising the steps of:
(a) coupling, in the presence of a tertiary amine and an acid activating reagent, 4-amino-5-(ethylsulfonyl)-2-methoxybenzoic acid and (S)-(1-ethylpyrrolidin-2-yl) methanamine to form a reaction mixture; and
(b) isolating from the reaction mixture of step (a), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°; wherein step (b) does not comprise formation of a solvate of (S)-(−)-amisulpride.

Provided herein is a method of preparing an enantiomerically pure crystalline form of (R)-(+)-amisulpride comprising the steps of:
(a) reacting 4-amino-5-(ethylsulfonyl)-2-methoxybenzoic acid with a tertiary amine and an acid activating reagent to form a reaction mixture;
(b) adding a (R)-(1-ethylpyrrolidin-2-yl)methanamine salt to the reaction mixture to form a mixture of step (b); and
(c) isolating from the mixture of step (b), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2°.

Also provided herein is a method of preparing an enantiomerically pure crystalline form of (S)-(−)-amisulpride comprising the steps of:
(a) reacting 4-amino-5-(ethylsulfonyl)-2-methoxybenzoic acid with a tertiary amine and an acid activating reagent to form a reaction mixture;
(b) adding a (S)-(1-ethylpyrrolidin-2-yl)methanamine salt to the reaction mixture of step (a) to form a mixture of step (b); and
(c) isolating from the mixture of step (b), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ), at least at 7.0±0.2° and 9.7±0.2°, and further peaks at 15.4±0.2° and/or 19.4±0.2°.

Also provided herein is a method of preparing an enantiomerically pure crystalline form of (R)-(+)-amisulpride comprising the steps of:
(a) heating (R)-(+)-amisulpride (e.g., crude, amorphous, or Form A of (R)-(+)-amisulpride) in the presence of an isolation reagent to a first elevated temperature to form a crystallization mixture;
(b) adding a seed amount of Form A of (R)-(+)-amisulpride to the crystallization mixture to form a seeded mixture;
(c) cooling the seeded mixture to a first reduced temperature over a first period of time;
(d) cooling the seeded mixture from step (c) to a second reduced temperature over a second period of time; and
(e) filtering the seeded mixture from step (d) to provide an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°.

Also provided herein is a method of preparing an enantiomerically pure crystalline form of (S)-(−)-amisulpride comprising the steps of:
(a) heating (S)-(−)-amisulpride (e.g., crude, amorphous, or Form A′ of (S)-(−)-amisulpride) in the presence of an isolation reagent to a first elevated temperature to form a crystallization mixture;
(b) adding a seed amount of a Form A′ of (S)-(−)-amisulpride to the crystallization mixture to form a seeded mixture;
(c) cooling the seeded mixture to a first reduced temperature over a first period of time;
(d) cooling the seeded mixture from step (c) to a second reduced temperature over a second period of time; and
(e) filtering the seeded mixture from step (d) to provide an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°.

The methods provided herein demonstrate good scalability (e.g., from gram to kilogram scales) and manufacturing convenience, providing crystalline products which are stable, with high yields of enantiomerically pure (R)-amisulpride and (S)-amisulpride. Further, the provided methods are safe, cost-effective, simplified, environmentally-friendly, and efficient (in both time and atom).

Provided herein is a pharmaceutical composition comprising the enantiomerically pure crystalline form of (R)-(+)-amisulpride or (S)-(−)-amisulpride prepared by a method disclosed herein, and a pharmaceutically acceptable carrier.

Provided herein is a method of treating a psychiatric disorder in a subject comprising administering to the subject an enantiomerically pure crystalline form of (R)-(+)-amisulpride or (S)-(−)-amisulpride prepared by a method disclosed herein, or an enantiomerically pure crystalline form of (R)-(+)-amisulpride or (S)-(−)-amisulpride disclosed herein, or a pharmaceutical composition disclosed herein. Provided herein is a method of treating a psychiatric disorder in a subject comprising administering to the subject an enantiomerically pure crystalline form of (R)-(+)-amisulpride and/or (S)-(−)-amisulpride prepared by a method disclosed herein, or an enantiomerically pure crystalline form of (R)-(+)-amisulpride and/or (S)-(−)-amisulpride disclosed herein, or a pharmaceutical composition disclosed herein.

### BRIEF DESCRIPTION OF THE DRAWINGS

In the accompanying drawings like reference numerals indicate like elements and features in the various figures. For clarity, not every element may be labeled in every figure. In addition, the drawings are not necessarily complete when viewed without reference to the text, emphasis instead being placed upon illustrating the principles of the inventions.

FIGS. 1A and 1B present various analytical data and images for crystalline (R)-amisulpride of Form A taken from and made according to U.S. Pat. No. 10,800,738 B2 (the '738 patent) where they respectively appear as FIGS. 2B and 2C. FIG. 1A presents an XRPD pattern for crystalline (R)-amisulpride of Form A. FIG. 1B presents a polarized light microscopy image of crystalline (R)-amisulpride of Form A. In FIG. 1A the 2-theta angle in degrees (x-axis) is plotted against peak intensity in terms of the count rate per second (y-axis).

FIGS. 2A and 2B present various analytical data and images for crystalline (S)-amisulpride of Form A′, taken from and made according to the '738 patent where they respectively appear as FIGS. 3B and 3C. FIG. 2A presents an XRPD pattern for crystalline (S)-amisulpride of Form A′. FIG. 2B presents a polarized light microscopy image an XRPD pattern for crystalline (S)-amisulpride of Form A′. In FIG. 2A the 2-Theta angle in degrees (x-axis) is plotted against peak intensity in terms of the count rate per second (y-axis).

FIG. 3 presents particle size distribution data (PSD) for the (R)-amisulpride of Form A and (S)-amisulpride of Form A′ of Example 9 after delumping.

FIG. 4A presents a polarized light microscopy (PLM) image of (S)-amisulpride of Form A′ of Example 9. FIG. 4B presents a PLM image of (R)-amisulpride of Form A of Example 9.

FIGS. 5A, 5B, 5C and 5D present scanning electron microscopy (SEM) images of the (S)-amisulpride of Form A′ and the (R)-amisulpride of Form A of Example 9. FIG. 5A presents an SEM image of (S)-amisulpride of Form A′ at ×100. FIG. 5B presents an SEM image of (R)-amisulpride of Form A at ×100. FIG. 5C presents an SEM image of (S)-amisulpride of Form A′ at ×200. FIG. 5D presents an SEM image of (R)-amisulpride of Form A at ×200.

FIG. 6A presents the XRPD spectrum for (R)-amisulpride of Form A prepared from Example 5. FIG. 6B presents the XRPD spectrum for (S)-amisulpride of Form A′ prepared from Example 4. The XRPD peaks are shown in Table 8 in Example 9.

FIG. 7A presents the overlay of dynamic vapor sorption (DVS) isotherms for (R)-amisulpride of Form A and (S)-amisulpride of Form A′ of Example 9. FIG. 7B presents the DVS isotherms for (R)-amisulpride of Form A. FIG. 7C presents the DVS isotherms for (S)-amisulpride of Form A′.

FIG. 8 presents differential scanning calorimetry (DSC) thermograms for (R)-amisulpride of Form A and (S)-amisulpride of Form A′ of Example 9.

FIG. 9 presents the XRPD spectrum for (R)-amisulpride of Form A prepared from Example 8.

FIG. 10 presents a plot of the cooling profile comparison between the cooling profile X, using the recrystallization procedure of Example 16, with cooling profile Y, using the recrystallization procedure of Example 4 (Step 2).

FIG. 11 presents a plot of the cumulative frequency (%) versus particle size in the tablet manufacturing process.

FIG. 12 presents a summary of the physical properties of amisulpride core tablets.

FIG. 13A presents a plot of the correlation between tablet thickness and hardness of 200 mg core tablets comprising (R)-(+)-amisulpride and (S)-(−)-amisulpride. FIG. 13B presents a plot of the correlation between tablet thickness and hardness of 100 mg core tablets comprising (R)-(+)-amisulpride and (S)-(−)-amisulpride.

### DETAILED DESCRIPTION

All published documents cited herein are hereby incorporated herein by reference in their entirety.

Reference in the specification to “one embodiment,” “an embodiment,” “one aspect,” or “an aspect” means that a particular, feature, structure or characteristic described in connection with the embodiment or aspect is included in at least one embodiment or aspect of the teachings.

As used herein, the singular forms “a”, “an” and “the” are intended to include the plural forms as well, unless the context clearly indicates otherwise. As used herein, the terms “comprising” and “including” or grammatical variants thereof are to be taken as specifying the stated features, integers, steps or components but do not preclude the addition of one or more additional features, integers, steps, components or groups thereof.

As used herein, and unless otherwise specified, the term “about”, when used in connection with a numeric value or range of values which is provided to describe a particular solid form (e.g., a specific temperature or temperature range, such as describing a melting, dehydration, or glass transition; a mass change, such as a mass change as a function of temperature or humidity; a solvent or water content, in terms of, for example, mass or a percentage; or a peak position, such as in analysis by, for example, ¹³C NMR, DSC, TGA and XRPD), indicate that the value or range of values may deviate to an extent deemed reasonable to one of ordinary skill in the art while still describing the particular solid form. For example, temperature readings in connection with DSC, TGA, or other thermal experiments can vary about ±3° C. depending on the instrument, particular settings, sample preparation, etc. The term “about”, when used in reference to a degree 2-theta value refers to ±0.2 degrees 2-theta. The term “about”, when used in reference to a temperature, refers to a temperature of 3° C. The term “about”, when used in other context, indicates that the numeric value or range of values may vary by ±5%, 4%, 3%, 2%, 1%, 0.9%, 0.8%, 0.7%, 0.6%, 0.5%, 0.4%, 0.3%, 0.2% or 0.1% of the recited value or range of values. For example, the term “about,” when used in the context of, e.g., mass, weight, or PSD values, indicates that the numeric value or range of values may vary by ±20%, 15%, 10%, 5%, 4%, 3%, 2%, 1%, 0.9%, 0.8%, 0.7%, 0.6%, 0.5%, 0.4%, 0.3%, 0.2% or 0.1% of the recited value or range of values.

As used herein, the term “substantially” when referring to a characteristic figure of a crystal form, such as an XRPD pattern, a DSC thermogram, a TGA thermogram, or the like, means that a subject figure can be non-identical to the reference depicted herein, but it falls within the limits of experimental error and thus can be deemed as derived from the same crystal form as disclosed herein, as judged by a person of ordinary skill in the art. For example, the term “substantially” as used in the context of XRPD herein is meant to encompass variations disclosed herein and known in the art.

In various embodiments the methods herein provide, (R)-amisulpride and (S)-amisulpride that are substantially crystalline. The term “substantially crystalline,” means a majority of the weight of a sample or preparation (e.g., of (R)-amisulpride or (S)-amisulpride) is crystalline and the remainder of the sample is a non-crystalline form (e.g., amorphous form) of the same compound. In various embodiments, a substantially crystalline sample has at least about 95% crystallinity and less than about 5% of the non-crystalline form of the same compound; at least about 96% crystallinity and less than about 4% of the non-crystalline form of the same compound; at least about 97% crystallinity and less than about 3% of the non-crystalline form of the same compound; at least about 98% crystallinity and less than about 2% of the non-crystalline form of the same compound; at least about 99% crystallinity and less than about 1% of the non-crystalline form of the same compound; and about 100% crystallinity and less than about 0% of the non-crystalline form of the same compound. The term “fully crystalline” means at least about 99% or at least about 100% crystallinity. In various embodiments, a substantially crystalline sample has at least about 95% crystallinity by weight and less than about 5% by weight of the non-crystalline form of the same compound; at least about 96% crystallinity by weight and less than about 4% by weight of the non-crystalline form of the same compound; at least about 97% crystallinity by weight and less than about 3% by weight of the non-crystalline form of the same compound; at least about 98% crystallinity by weight and less than about 2% of the non-crystalline form by weight of the same compound; at least about 99% crystallinity by weight and less than about 1% by weight of the non-crystalline form of the same compound; or about 100% crystallinity by weight of the non-crystalline form of the same compound. The term “fully crystalline” means at least about 99% by weight or at least about 100% crystallinity by weight.

The term “crystalline form,” or grammatical variants thereof, is meant herein to refer to a certain lattice configuration of a crystalline substance. Different crystalline forms of the same substance typically have different crystalline lattices (e.g., unit cells), typically have different physical properties attributed to their different crystalline lattices. The different crystalline lattices can be identified by solid state characterization methods such as XRPD. Other characterization methods such as differential scanning calorimetry (DSC), thermogravimetric analysis (TGA), dynamic vapor sorption (DVS), and the like further help identify the crystalline form as well as help determine stability and solvent/water content.

The term “solvate” refers to a crystal form where a stoichiometric or non-stoichiometric amount of solvent, or mixture of solvents, is incorporated into the crystal structure. In certain embodiments, the solvate is a hydrate. The term “hydrate” refers to a crystal form where a stoichiometric or non-stoichiometric amount of water is incorporated into the crystal structure. In some embodiments, the solvate is an ethyl acetate solvate.

The term “amorphous” or “amorphous form” is intended to mean that the substance, component, or product in question is not crystalline as determined, for instance, by XRPD or where the substance, component, or product in question, for example is not birefringent when viewed microscopically. For example, amorphous means essentially without regularly repeating arrangement of molecules or lacks the long range order of a crystal, i.e., amorphous form is non-crystalline. An amorphous form does not display a defined x-ray diffraction pattern with sharp maxima. In certain embodiments, a sample comprising an amorphous form of a substance can be substantially free of other amorphous forms and/or crystalline forms. For example, an amorphous substance can be identified by an XRPD spectrum having an absence of readily distinguishable reflections.

The terms “% crystallinity” and “crystalline purity” are used interchangeably and refer to the weight % that is the specified crystalline form. For example, when a crystalline (R)-amisulpride of Form A is characterized as having greater than 95% crystalline purity, that means that greater than 95% by weight of the substance is crystalline (R)-amisulpride of Form A and less than 5% by weight of any other crystalline form or amorphous form of (R)-amisulpride.

The terms “chiral purity” and “enantiomeric purity” are used interchangeably and refer to a measurement of purity for a chiral compound as the weight % that is the specified enantiomer. The measurement can be determined by methods well-known in the art, e.g., by specific optical rotation, chiral column chromatography, NMR spectroscopy, and the like. For example, when a (R)-amisulpride containing substance (such as a compound or crystal form) is characterized as having greater than 90% enantiomeric purity, that means that greater than 90% by weight of the amisulpride in the substance is the (R)-amisulpride and less than 10% by weight is in any other enantiomeric form of amisulpride.

The term “chemical purity” refers to the weight % that is the specified chemical entity, including specified crystalline form. For example, when a crystalline amisulpride Form A′ is characterized as having greater than 95% chemical purity, that means that greater than 95% by weight of the substance is crystalline amisulpride Form A′ and less than 5% by weight of other compounds.

As used herein, the term “crystalline (R)-amisulpride Form A,” “crystalline form of (R)-(+)-amisulpride,” “crystalline Form A of (R)-amisulpride,” “Form A,” and “(R)-amisulpride Form A” are used interchangeably. Further, the term “crystalline (S)-amisulpride Form A′,” “crystalline form of (S)-(−)-amisulpride,” “crystalline Form A′ of (S)-amisulpride,” “Form A′,” and “(S)-amisulpride Form A′” are used interchangeably.

For example, when a crystalline (R)-amisulpride Form A is characterized as having greater than 99% chemical purity and greater than 97% chiral purity, that means greater than 97% by weight of the substance is of enantiomeric form (R)-amisulpride Form A and less than 3% by weight of any other amisulpride enantiomer, and that greater than 99% by weight of the substance is amisulpride and less than 1% by weight of other compounds. For example, when a crystalline (R)-amisulpride Form A is characterized as having greater than 99% chemical purity, greater than 97% chiral purity and greater than 95% crystalline purity, that means that greater than 95% by weight of the substance is crystalline (R)-amisulpride of Form A and less than 5% by weight of any other crystalline or amorphous form of (R)-amisulpride, greater than 97% by weight of the substance is of enantiomeric form (R)-amisulpride and less than 3% by weight of any other amisulpride enantiomer, and that greater than 99% by weight of the substance is amisulpride and less than 1% by weight of other compounds.

For example, when a crystalline (S)-amisulpride Form A′ is characterized as having greater than 99% chemical purity and greater than 97% chiral purity, that means greater than 97% by weight of the substance is of enantiomeric form (S)-amisulpride Form A′ and less than 3% by weight of any other amisulpride enantiomer, and that greater than 99% by weight of the substance is amisulpride and less than 1% by weight of other compounds. For example, when a crystalline (S)-amisulpride Form A′ is characterized as having greater than 99% chemical purity, greater than 97% chiral purity and greater than 95% crystalline purity, that means that greater than 95% by weight of the substance is crystalline (S)-amisulpride of Form A′ and less than 5% by weight of any other crystalline or amorphous form of (S)-amisulpride, greater than 97% by weight of the substance is of enantiomeric form (S)-amisulpride and less than 3% by weight of any other amisulpride enantiomer, and that greater than 99% by weight of the substance is amisulpride and less than 1% by weight of other compounds.

Crystal forms of amisulpride, enantiomeric amisulpride, and crystalline forms of their salts, hydrates and solvates, may be characterized and differentiated using a number of conventional analytical techniques, including but not limited to XRPD patterns, NMR spectra, Raman spectra, Infrared (IR) absorption spectra, dynamic vapor sorption (DVS), differential scanning calorimetry (DSC), and melting point. Chemical purity may be characterized using a number of conventional analytical techniques, including but not limited to high performance liquid chromatography (HPLC), gas chromatography (GC), mass spectrometry (MS), NMR, and HPLC/MS. Chiral purity (also known as enantiomeric purity) may be characterized using a number of conventional analytical techniques, including but not limited to chiral chromatography, chiral HPLC, and NMR.

In various embodiments, the crystal forms of racemic amisulpride provided by the methods herein are characterized by XRPD. XRPD is a technique of characterizing a powdered sample of a material by measuring the diffraction of X-rays by the material. The result of an XRPD experiment is a diffraction pattern. Each crystalline solid produces a distinctive diffraction pattern containing sharp peaks as a function of the scattering angle 20 (2-theta). Both the positions (corresponding to lattice spacing) and the relative intensity of the peaks in a diffraction pattern are indicative of a particular phase and material. This provides a “fingerprint” for comparison to other materials. In contrast to a crystalline pattern comprising a series of sharp peaks, amorphous materials (liquids, glasses etc.) produce a broad background signal in a diffraction pattern. As used herein, the term “peak” or “characteristic peak,” in the context of an XRPD pattern, refers to a reflection having a relative height/intensity of at least about 5% of the maximum peak height/intensity.

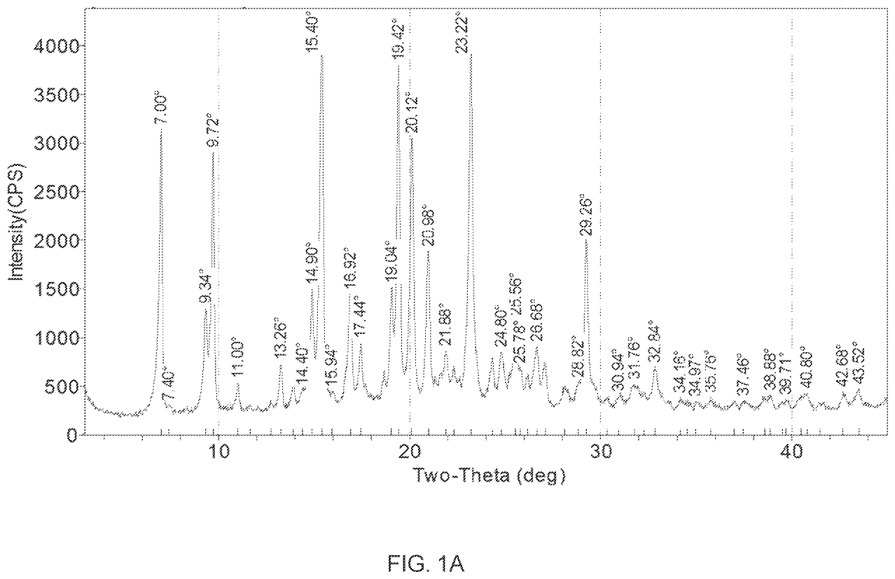

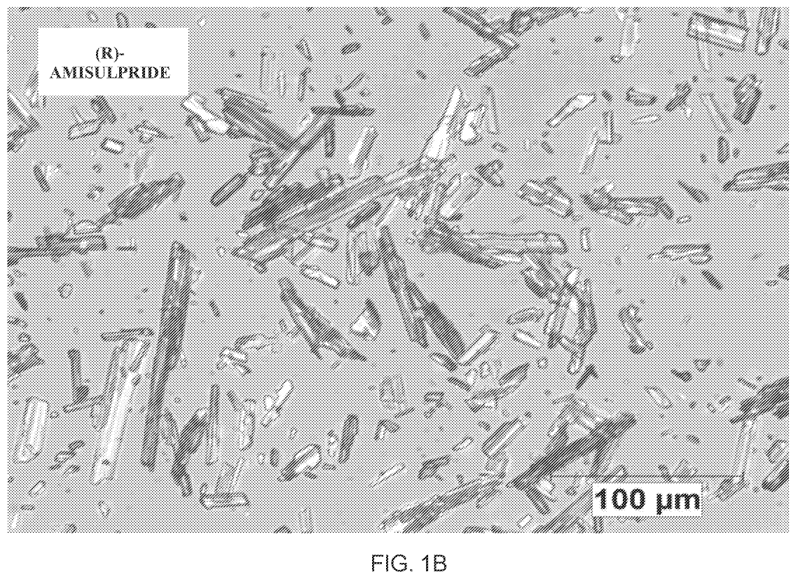

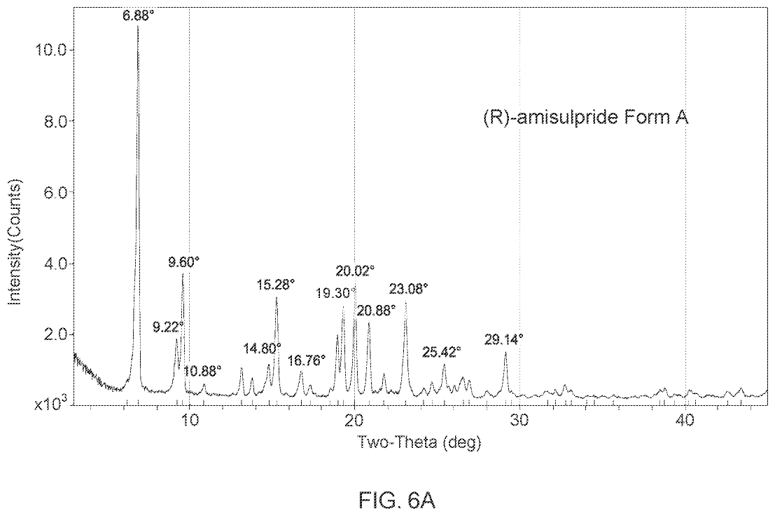

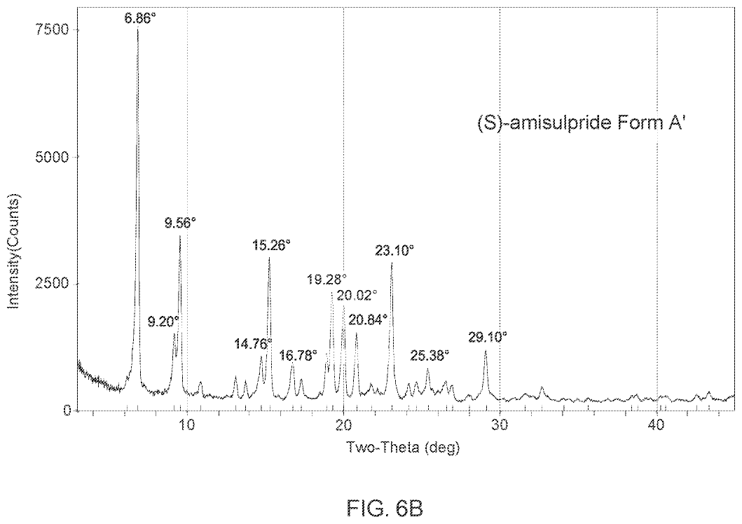

It is to be understood that with the XRPD apparatus employed, humidity, temperature, orientation of the powder crystals, filters used, and other parameters involved in obtaining an XRPD pattern may cause some variability in the appearance, intensities, and positions of the lines in the diffraction pattern. An XRPD pattern that is “substantially in accord with” that of a FIG. provided herein (e.g., FIG. 1A, FIG. 6A, FIG. 6B, etc.) is an XRPD pattern that would be considered by one skilled in the art to represent a compound possessing the same crystal form as the compound that provided the XRPD pattern of that FIG. That is, the XRPD pattern may be identical to that of the FIG., or more likely it may be somewhat different. Such an XRPD pattern may not necessarily show each of the lines of the diffraction patterns presented herein, and/or may show a slight change in appearance, intensity, or a shift in position of said lines resulting from differences in the conditions involved in obtaining the data. Moreover, instrument variation and other factors can affect the 2-theta (2θ) values. Thus, peak assignments, such as those reported herein, can vary by plus or minus (±) about 0.2° (2-theta). A person skilled in the art is capable of determining if a sample of a crystalline compound has the same form as, or a different form from, a form disclosed herein by comparison of their XRPD patterns.

For example, one skilled in the art could use HPLC to determine the enantiomeric identity of an amisulpride sample and if, for example, the sample is identified as (R)-amisulpride, one skilled in the art can overlay an XRPD pattern of the amisulpride sample with FIG. 1A and/or the appropriate FIG. 6A or FIG. 6B, and using expertise and knowledge in the art, readily determine whether the XRPD pattern of the sample is substantially in accordance with the XRPD pattern of crystalline (R)-amisulpride of Form A presented in FIG. 1A and/or FIG. 6A. If, for example, HPLC identifies the sample as being (R)-amisulpride and the sample XRPD pattern is substantially in accord with FIG. 1A and/or FIG. 6A, the sample can be readily and accurately identified as (R)-amisulpride of Form A.

In various embodiments, the crystal forms of enantiomeric amisulpride can be further distinguished from amorphous amisulpride by melting point, in addition to XRPD. Melting points were determined by conventional methods such as capillary tube and may exhibit a range over which complete melting occurs, or in the case of a single number, a melting point of that temperature±3° C.

In various embodiments, the hygroscopicity of a compound is characterized by dynamic vapor sorption (DVS). DVS is a gravimetric technique that measures how much of a solvent is absorbed by a sample by varying the vapor concentration surrounding the sample (e.g., relative humidity) and measuring the change in mass. DVS was used herein to generate water sorption isotherms, which represent the equilibrium amount of water vapor adsorbed and/or absorbed as a function of steady state relative water vapor pressure at a constant temperature.

The acid activating reagent is a reagent to facilitate the coupling of carboxylic acid with the desired amine. The carboxylate reacts with the activating reagent to form a reactive intermediate (e.g., a carboxylic anhydride). Examples of suitable acid activating reagents include, but are not limited to, alkyl chloroformate, acid halides (e.g., chloride, fluoride), triazines, anhydrides, or carbodiimides. In certain embodiments, the acid activating reagent is an alkyl chloroformate (e.g., methyl chloroformate, and ethyl chloroformate). In certain embodiments, the acid activating reagent is of the formula

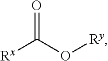

wherein R^{x} is halogen and R^{y} is C₁₋₅ alkyl.

The term “substantially non-hygroscopic” refers to a compound exhibiting less than a 1% maximum mass change in water sorption isotherms, at 25° C. scanned over 0 to 95% relative humidity, as measured by dynamic vapor sorption (DVS).

The following abbreviations are used herein. The abbreviation DSC refers to differential scanning calorimetry; the abbreviation XRPD refers to x-ray powder diffraction, the abbreviation NMR refers to nuclear magnetic resonance, the abbreviation DVS refers to, dynamic vapor sorption, the abbreviation HPLC refers to high performance liquid chromatography, the abbreviation GC refers to gas chromatography, the abbreviation SSA refers to specific surface area, and the abbreviation TGA refers to thermogravimetric analysis. The abbreviations (R)-(+)-amisulpride and (R)-amisulpride refer to (R)-4-Amino-N-[(L-ethyl-2-pyrrolidinyl)methyl]-5-(ethylsulfonyl)-2-methoxybenzamide. The abbreviations (S)-(−)-amisulpride and (S)-amisulpride refer to (S)-4-Amino-N-[(1-ethyl-2-pyrrolidinyl)methyl]-5-(ethylsulfonyl)-2-methoxybenzamide. Other abbreviations not explicitly described herein, and unless defined otherwise, all technical and scientific terms used herein have the same meaning as commonly understood by one of ordinary skill in the art.

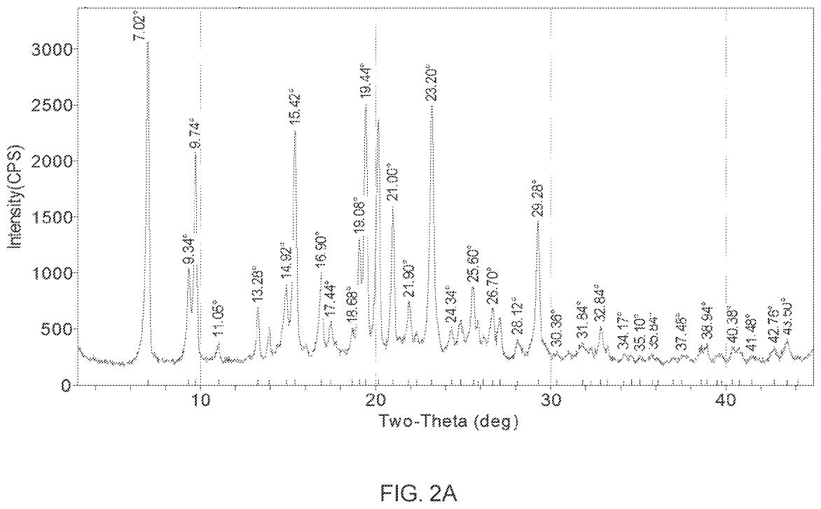

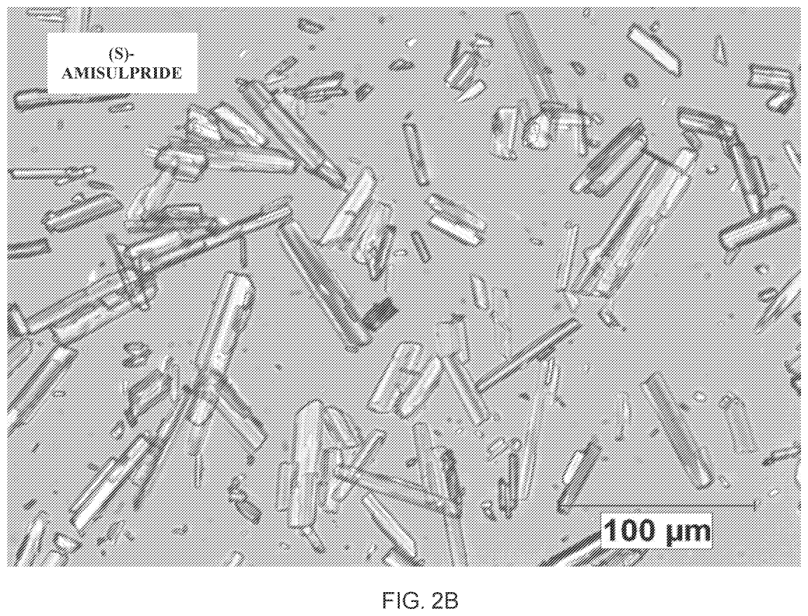

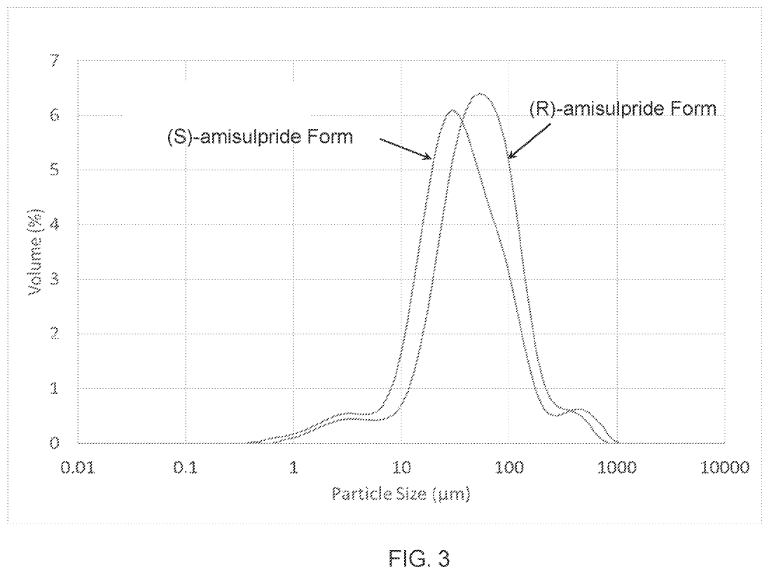

The disclosures herein provide new methods for the preparation of crystalline forms of enantiomeric amisulpride of Form A and Form A′. Forms A and A′ have been previously characterized and are described in U.S. Pat. No. 10,800,738 B2 (the '738 patent). Table 1 summarizes various previously determined properties and data on Form A crystals of (R)-amisulpride and Form A′ crystals of (S)-amisulpride found in '738 patent. The FIG. references in Table 1 are to figures in the present application, where FIGS. 1A and 1B in the present application appear as FIGS. 2A and 2C in the '738 patent, and FIGS. 2A and 2B in the present application appear as FIGS. 3A and 3C in the '738 patent.

TABLE 1

Compound

| | (R)-amisulpride | (S)-amisulpride |
|---|---|---|
| Physical Property | Form A | Form A′ |
| Melting Point, ° C. | 102 | 102 |
| XRPD Pattern | FIG. 1A | FIG. 2A |
| Micrograph Image | FIG. 1B | FIG. 2B |

Provided herein are methods for preparing amisulpride and crystalline forms thereof, including methods for making (R)-amisulpride and (S)-amisulpride, independently, in a free base crystal form, and thus substantially without any water or solvent incorporated into the crystal structure. In various aspects, the methods provide enantiomerically pure (S)-amisulpride and (R)-amisulpride, and crystalline forms thereof, with high chemical purity.

Crystalline forms of enantiomerically pure amisulpride and methods for making the same, are described in U.S. Pat. No. 10,800,738 B2 (the '738 patent) the entirety of which is incorporated herein by reference. The '738 patent describes several crystalline forms of amisulpride including enantiomerically pure free base crystalline forms of (R)-amisulpride and (S)-amisulpride, and thus crystalline forms without any water or solvent incorporated into the crystal structure. These crystalline forms, which for the sake of concise identification therein, and herein, are referred to as Form A for the free base crystalline form of (R)-amisulpride, and Form A′ for the free base crystalline form of (S)-amisulpride.

In the '738 patent these free base crystalline Forms A and A′ were shown to have several advantageous physical properties including being substantially non-hygroscopic, exhibiting less than a 0.5% maximum mass change in water sorption isotherms, at 25° C. scanned over 0 to 95% relative humidity, as measured by dynamic vapor sorption (DVS).

Producing high yields of a specific crystalline form, and thus high purity of that crystalline form, is often limited by the formation of amorphous products and other crystalline forms that may, for example, be kinetically favored. The '738 patent discovered that making crystalline enantiomeric amisulpride was complicated by the fact that traditional methods resulted in non-crystalline (amorphous) enantiomeric amisulpride.

The methods provided in the '738 patent for making Form A and Form A′ of enantiomeric amisulpride required formation of an intermediate amisulpride solvate (for example, an ethyl acetate solvate) followed by conversion to the free base to obtain the crystalline Forms A and A′. In contrast to the methods described in the '738 patent, the methods provided herein, among other things, do not require the formation of an amisulpride solvate as described in the '738 patent. For example, the methods provided herein do not generate an intermediate amisulpride solvate (e.g., an intermediate solvate of (R)-amisulpride such as an ethyl acetate solvate of (R)-amisulpride, or an intermediate solvate of (S)-amisulpride such as an ethyl acetate solvent of (S)-amisulpride). Accordingly, the methods provided herein avoid the extra step of converting the solvated form of amisulpride to the non-solvated form. Therefore, the methods provided herein demonstrate good scalability (e.g., from gram to kilogram scales) and manufacturing convenience, providing crystalline products which are stable, with high yields of enantiomerically pure (R)-amisulpride and (S)-amisulpride. Further, the provided methods are safe, cost-effective, simplified, environmentally-friendly, and efficient (in both time and atom).

The methods provided herein for making Form A and Form A′ also includes formation of the crystalline solid having certain particle size that provides a narrower and smaller particle size distribution (PSD) compared to other crystallization procedures. Having a narrower and smaller PSD can improve the manufacturability, controllability, and production workflows of formulation manufacturing (e.g., tablet manufacturing). In particular, although milling and/or sizing processes can be used to control pharmaceutical product size in general, amisulpride cannot be easily milled because it has a relatively low melting point and can melt during the milling process, sticking to milling equipment. Thus, by having a small particle size distribution to start, manufacturing is facilitated. In addition, it was found that tablets containing Form A and Form A′ having smaller PSD have higher hardness values, compared to tablets containing crystalline Form A and Form A′ having larger PSD.

Moreover, certain methods provided herein provide a recrystallization procedure using a solvent system that is a mixture of an isolation reagent (e.g., isopropyl acetate) and a solvent, S5, wherein (R) and/or (S)-amisulpride is less soluble in S5 than in the isolation reagent. The inclusion of S5 in the solvent system can allow for better mixing without loss of yield in the final product compared to recrystallization procedures that do not use S5, thereby increasing manufacturing ease and convenience.

As used in the context of the methods described herein, the term “Form A” refers to a crystalline form of (R)-(+)-amisulpride having a powder x-ray crystal pattern comprising peaks, when measured using CuKα radiation, in terms of 2-theta, at least at 7.0±0.2° and 9.7±0.2°, and further peaks at 15.4±0.2° and/or 19.4±0.2°; and optionally with additional peaks, when measured using CuKα radiation, in terms of 2-theta, at two or more of: 9.3±0.2°, 14.9±0.2°, 16.9±0.2°, 19.0±0.2°, 19.4±0.2°, 20.1±0.2°, 21.0±0.2°, 23.2±0.2°, and 29.3±0.2°; and in various embodiments an powder x-ray crystal pattern substantially in accord with one or more of FIG. 1A and FIG. 6A.

As used in the context of the methods described herein, the term “Form A′” refers to a method that produces a crystalline form of (S)-(−)-amisulpride having a powder x-ray crystal pattern comprising peaks, when measured using CuKα radiation, in terms of 2-theta, at least at 7.0±0.2° and 9.7±0.2°, and further peaks at 15.4±0.2° and/or 19.4±0.2°; and optionally with additional peaks, when measured using CuKα radiation, in terms of 2-theta, at two or more of: 9.3±0.2°, 14.9±0.2°, 16.9±0.2°, 19.0±0.2°, 19.4±0.2°, 20.1±0.2°, 21.0±0.2°, 23.2±0.2°, and 29.3±0.2°; and in various embodiments an powder x-ray crystal pattern substantially in accord with one or more of FIG. 2A and FIG. 6B.

Various embodiments of the methods described herein provide crystalline forms of amisulpride enantiomers of Form A and Form A′, that have high crystal form, enantiomeric and/or chemical purity.

Tables 2-5 present further information from the '738 patent providing details on XRPD patterns of FIGS. 1A and 2A herein (FIGS. 2B and 3B respectively in the '738 patent) to facilitate comparisons and characterization of the crystalline forms of enantiomeric amisulpride prepared by the new methods provided herein to the Form A and Form A′ crystalline forms described in the '738 patent. Tables 2, 3, 4 and 5 herein are adapted, respectively, from Tables 2, 3, 4 and 5 of the '738 patent.

In addition, Example 9 presents information and data on crystalline forms of enantiomeric amisulpride prepared according to various embodiments of the methods provided herein.

### Crystals of (R)-Amisulpride Form A

TABLE 2
(R)-amisulpride Form A Single Crystal
Data and Data Collection Parameters

| Empirical formula | C₁₇H₂₇N₃O₄S |
|---|---|
| Molecular weight (g mol⁻¹) | 369.47 |
| Crystal system | triclinic |
| Space group | P1 |
| Unit cell parameters | |
| a = 12.3348(4) Å | α = 64.033(4)° |
| b = 12.8343(6) Å | β = 73.431(3)° |
| c = 14.1403(6) Å | γ = 75.881(3)° |
| Unit cell volume (Å³) | 1910.47(15) |
| Cell formula units, Z | 4 |
| Calculated density (g cm⁻³) | 1.285 |
| Absorption coefficient (mm⁻¹) | 1.728 |
| F(000) | 792 |
| Crystal size (mm³) | 0.25 × 0.04 × 0.02 |

TABLE 3
(R)-amisulpride Form A XRPD (FIG. 1A) Peak List

| 2-Theta | Relative Height |
|---|---|
| 7.00 | 75 |
| 7.42 | 1.6 |
| 9.34 | 26.9 |
| 9.72 | 68.3 |
| 9.95 | 1.5 |
| 11.00 | 6.7 |
| 11.66 | 1.2 |
| 12.72 | 2.3 |
| 13.26 | 11.3 |
| 13.90 | 5.2 |
| 14.41 | 4.8 |
| 14.72 | 13.5 |
| 14.90 | 31 |
| 15.40 | 100 |
| 15.94 | 4 |
| 16.64 | 7.9 |
| 16.92 | 28 |
| 17.44 | 14.8 |
| 17.70 | 4 |
| 18.66 | 7.5 |
| 19.04 | 29.3 |
| 19.42 | 87 |
| 20.12 | 63.7 |
| 20.98 | 34.8 |
| 21.62 | 3.5 |
| 21.88 | 7.8 |
| 22.32 | 3.8 |
| 22.61 | 2.5 |
| 23.22 | 89.3 |
| 24.34 | 8.1 |
| 24.80 | 8.7 |
| 25.26 | 3 |
| 25.56 | 17 |
| 25.78 | 4.3 |
| 26.20 | 3.2 |
| 26.68 | 15.8 |
| 27.10 | 11.3 |
| 28.12 | 3.5 |
| 28.28 | 2.6 |
| 28.82 | 5.2 |
| 29.26 | 42.2 |
| 29.56 | 5.9 |
| 29.76 | 3.7 |
| 30.32 | 1.9 |
| 30.92 | 1.7 |
| 31.02 | 2.6 |
| 31.70 | 4.3 |
| 31.94 | 3.8 |
| 32.26 | 2.2 |
| 32.84 | 8.9 |
| 33.22 | 2.7 |
| 34.16 | 2.7 |
| 34.55 | 2.2 |
| 34.97 | 1.7 |
| 35.24 | 1.1 |
| 35.48 | 0.9 |
| 35.76 | 2.9 |
| 37.00 | 1.9 |
| 37.44 | 1.3 |
| 38.58 | 3.2 |
| 38.88 | 3.4 |
| 39.50 | 1.6 |
| 39.76 | 2.1 |
| 40.38 | 2.5 |
| 40.80 | 3.7 |
| 41.39 | 1.4 |
| 41.68 | 1.5 |
| 42.68 | 3.7 |
| 43.28 | 2.8 |
| 43.52 | 4.7 |

### Crystals of (S)-Amisulpride Form A′

TABLE 4
(S)-amisulpride Form A′ Single Crystal
Data and Data Collection Parameters

| Empirical formula | C₁₇H₂₇N₃O₄S |
|---|---|
| Formula weight (g mol⁻¹) | 369.47 |
| Crystal system | triclinic |
| Space group | P1 |
| Unit cell parameters | |
| a = 12.3795(4) Å | α = 64.246(3)° |
| b = 12.7526(4) Å | β = 73.598(3)° |
| c = 14.1438(4) Å | γ = 75.797(3)° |
| Unit cell volume (Å³) | 1909.71(11) |
| Cell formula units, Z | 4 |
| Calculated density (g cm⁻³) | 1.285 |
| Absorption coefficient (mm⁻¹) | 1.728 |
| F(000) | 792 |
| Crystal size (mm³) | 0.2 × 0.04 × 0.02 |

TABLE 5
(S)-amisulpride Form A′ XRPD (FIG. 2A) Peak List

| 2-Theta | Relative Height |
|---|---|
| 7.02 | 100 |
| 9.34 | 28 |
| 9.74 | 62 |
| 11.05 | 5.6 |
| 13.28 | 15.2 |
| 13.94 | 7.8 |
| 14.92 | 20 |
| 15.42 | 66.2 |
| 16.90 | 23.9 |
| 17.44 | 8.9 |
| 18.68 | 7.4 |
| 19.08 | 34.2 |
| 19.44 | 74.4 |
| 20.16 | 70 |
| 21.00 | 41.2 |
| 21.90 | 12 |
| 22.36 | 3.1 |
| 23.20 | 72.1 |
| 24.34 | 5.7 |
| 24.87 | 7 |
| 25.60 | 16.9 |
| 25.84 | 6.2 |
| 26.17 | 2.3 |
| 26.70 | 14.8 |
| 27.12 | 12.1 |
| 28.12 | 5.2 |
| 29.28 | 40.4 |
| 30.36 | 2.2 |
| 31.84 | 3.8 |
| 32.30 | 2.4 |
| 32.84 | 9 |
| 33.26 | 3.7 |
| 34.17 | 2.5 |
| 34.64 | 2 |
| 35.10 | 1.8 |
| 35.84 | 2.8 |
| 36.14 | 1.6 |
| 37.00 | 1.6 |
| 37.48 | 2.1 |
| 38.60 | 4.8 |
| 38.94 | 5.2 |
| 39.52 | 1.6 |
| 39.75 | 2.1 |
| 40.38 | 4.1 |
| 40.76 | 4.2 |
| 41.48 | 1.8 |
| 42.76 | 3.6 |
| 43.50 | 5.7 |
| 44.12 | 1.1 |

The term “reacting,” “contacting” or “treating” when describing a certain process is used as known in the art and generally refers to the bringing together of chemical reagents in such a manner so as to allow their interaction at the molecular level to achieve a chemical or physical transformation. In some embodiments, the reacting involves two reagents, wherein one or more equivalents of second reagent are used with respect to the first reagent. The reacting steps of the processes described herein can be conducted for a time and under conditions suitable for preparing the identified product.

The reactions of the processes described herein can be carried out in air or under an inert atmosphere. Typically, reactions containing reagents or products that are substantially reactive with air can be carried out using air-sensitive synthetic techniques that are well known to the skilled artisan.

The term “seeding” refers to providing a seed crystal (e.g., a small amount of the desired product) and/or nucleation center. It is to be understood that it is preferred that the seed crystal be of the same enantiomeric confirmation as the desired product to minimize introduction of chiral impurity into the desired product. Further, the seed crystal can be of the same crystalline form as the desired product. In various embodiments, a nucleation center is sufficient as a seed to induce appropriate crystallization although one of ordinary skill in the art will understand that such nucleation centers are to be used in the lowest amounts practicable to minimize introduction of impurities in the final desired product. Without being bound to a particular theory, seeding is used to control the initial supersaturation consumption rate during crystallization, which could stimulate growth and minimize secondary nucleation risk leading to a final product with unimodal particle size distribution and improved product purity. The term “seeded mixture” refers to a mixture that has been seeded, e.g., a mixture in which seed crystals have been added in order to induce crystallization. The term “seed amount” can refer to the lowest amounts practicable to allow for seeding but minimize introduction of impurities in the final desired product. In some embodiments, the term “seed amount” refers to a total weight of the seed that is about 0.001 wt % to about 25 wt % of the mixture to which the seed is added, about 0.01 wt % to about 10 wt % of the mixture to which the seed is added, or about 0.01 wt % to about 5 wt % of the mixture to which the seed is added. In some embodiments, the term “seed amount” refers to a total weight of the seed that is about 0.4 wt %, 1.4 wt %, or about 2.0 wt % of the mixture to which the seed is added.

In some embodiments, the term “seed amount” refers to a total weight of the seed that is about 0.001 wt % to about 10 wt % of the expected yield of the (R)- or (S)-amisulpride present in the mixture to which the seed is added, about 0.001 wt % to about 5 wt % of the expected yield of the (R)- or (S)-amisulpride present in the mixture to which the seed is added, or about 0.01 wt % to about 5 wt % of the expected yield of the (R)- or (S)-amisulpride present in the mixture to which the seed is added. In some embodiments, the term “seed amount” refers to a total weight of the seed that is about 0.01 wt % to about 2.0 wt % of the expected yield of the (R)- or (S)-amisulpride present in the mixture to which the seed is added, about 0.01 wt % to about 10 wt % of the expected yield of the (R)- or (S)-amisulpride present in the mixture to which the seed is added. In some embodiments, the term “seed amount” refers to a total weight of the seed that is about 0.01 wt % to about 0.75 wt % of the expected yield of the (R)- or (S)-amisulpride present in the mixture to which the seed is added. In some embodiments, the term “seed amount” refers to a total weight of the seed that is about 0.4 wt %, 1.4 wt %, or about 2.0 wt % of the expected yield of the (R)- or (S)-amisulpride present in the mixture to which the seed is added. In some embodiments, the term “seed amount” refers to a total weight of the seed that is about 0.4 wt %, 0.75%, 1.4 wt %, or about 2.0 wt % of the expected yield of the (R)- or (S)-amisulpride present in the mixture to which the seed is added. In some embodiments, the term “seed amount” refers to a total weight of the seed that is about 0.75 wt % of the expected yield of the (R)- or (S)-amisulpride present in the mixture to which the seed is added.

The term “isolating” refers to the concentration and/or extraction of a compound within or from a solution, mixture, and/or phase. For example, isolating can include extraction of compound from one phase of a mixture (e.g. an organic phase in a mixture with water and organic phases), which alternatively may be viewed as removing one phase form the mixture (e.g. removal of water). Isolating may and can include, for example, distillation, precipitation, phase separation, crystallization, recrystallization, and the like.

The process and reactions of the methods described herein can be carried out in a range of solvents. A given reaction can be carried out in one solvent or a mixture of more than one suitable solvents. Depending on the particular reaction step, suitable solvents for a particular reaction step can be selected. Further, recrystallization can be carried out in one solvent or a mixture of more than one suitable solvents.

The term “halogenated solvents” includes, but is not limited to, solvents such as carbon tetrachloride, bromodichloromethane, dibromochloromethane, bromoform, chloroform, bromochloromethane, dibromomethane, dichloromethane (methylene chloride), butyl chloride, tetrachloroethylene, trichloroethylene, 1,1,1-trichloroethane, 1,1,2-trichloroethane, 1,1-dichloroethane, 2-chloropropane, 1,1,1-trifluorotoluene, 1,2-dichloroethane, 1,2-dibromoethane, hexafluorobenzene, 1,2,4 trichlorobenzene, 1,2-dichlorobenzene, chlorobenzene, fluorobenzene, mixtures thereof and the like.

The term “ether solvents” includes, but is not limited to, solvents such as dimethoxymethane, 1,3-dioxane, 1,4-dioxane, furan, tetrahydrofuran (THF), diethyl ether, ethylene glycol dimethyl ether, ethylene glycol diethyl ether, diethylene glycol dimethyl ether (diglyme), diethylene glycol diethyl ether, triethylene glycol dimethyl ether, anisole, tert-butyl methyl ether, mixtures thereof and the like.

The term “protic solvents” (e.g., polar protic solvents) includes, but is not limited to, solvents such as water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, iso-butyl alcohol, tert-butyl alcohol, 2-ethoxyethanol, diethylene glycol, 1-, 2-, or 3-pentanol, neo-pentyl alcohol, tert-pentyl alcohol, diethylene glycol monomethyl ether, diethylene glycol monoethyl ether, cyclohexanol, benzyl alcohol, phenol, glycerol, mixtures thereof and the like.

The term “aprotic solvents” includes, but is not limited to, solvents such as N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, isopropyl acetate, hexachloroacetone, acetone, ethyl methyl ketone, ethyl acetate, sulfolane, N,N-dimethylpropionamide, tetramethylurea, nitromethane, nitrobenzene, hexamethylphosphoramide, mixtures thereof and the like.

The term “hydrocarbon solvents” includes, but is not limited to, solvents such as benzene, cyclohexane, pentane, hexane, toluene, cycloheptane, methylcyclohexane, heptane, ethylbenzene, m-, o-, or p-xylene, octane, indane, nonane, naphthalene, mixtures thereof and the like.

The processes described herein can be monitored according to any suitable method known in the art. For example, product formation can be monitored by spectroscopic means, such as nuclear magnetic resonance (NMR) spectroscopy (e.g., ¹H or ¹³C), infrared spectroscopy, spectrophotometry (e.g., UV-visible), mass spectrometry (MS); by chromatography such as high performance liquid chromatography (HPLC) and/or HPLC/MS. The purity of the compounds, in general, can be determined by, for example, polarimetry, NMR, HPLC/MS, and XRPD.

### Free Base Methods

Provided herein is a method of preparing an enantiomerically pure crystalline form of (R)-(+)-amisulpride comprising the steps of:
(a) coupling, in the presence of a tertiary amine and an acid activating reagent, 4-amino-5-(ethylsulfonyl)-2-methoxybenzoic acid and (R)-(1-ethylpyrrolidin-2-yl) methanamine to form a reaction mixture; and
(b) isolating from the reaction mixture of step (a), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°; wherein step (b) does not comprise formation of a solvate of (R)-(+)-amisulpride.

In some embodiments, the coupling of step (a) comprises the steps of:
(a1) reacting 4-amino-5-(ethylsulfonyl)-2-methoxybenzoic acid with the tertiary amine and the acid activating reagent to form a first reaction mixture; and
(a2) adding (R)-(1-ethylpyrrolidin-2-yl)methanamine to the first reaction mixture.

Also provided herein is a method of preparing an enantiomerically pure crystalline form of (S)-(−)-amisulpride comprising the steps of:
(a) coupling, in the presence of a tertiary amine and an acid activating reagent, 4-amino-5-(ethylsulfonyl)-2-methoxybenzoic acid and (S)-(1-ethylpyrrolidin-2-yl) methanamine to form a reaction mixture; and
(b) isolating from the reaction mixture of step (a), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°; wherein step (b) does not comprise formation of a solvate of (S)-(−)-amisulpride.

In some embodiments, the coupling of step (a) comprises the steps of:
(a1) reacting 4-amino-5-(ethylsulfonyl)-2-methoxybenzoic acid with the tertiary amine and the acid activating reagent to form a first reaction mixture; and
(a2) adding (S)-(1-ethylpyrrolidin-2-yl)methanamine to the first reaction mixture.

In some embodiments, the coupling of step (a) of preparing an enantiomerically pure crystalline form of (R)-(+)-amisulpride or (S)-(−)-amisulpride is carried out in the presence of S1, which is a solvent. In some embodiments, S1 is an aprotic solvent. In some embodiments, S1 is acetone. In some embodiments, S1 is a mixture of aprotic solvents.

In some embodiments, step (b) of the above methods comprises step (b1): concentrating the reaction mixture of step (a) to give a mixture of step (b1).

In some embodiments, step (b) further comprises step (b2): treating the mixture of step (b1) with a base and the isolation reagent to form a step (b2) mixture. In some embodiments, the base is an inorganic base. In some embodiments, the base is an alkali metal carbonate or an alkali metal hydroxide. In some embodiments, the base is an alkali metal carbonate (e.g., sodium carbonate, potassium carbonate, lithium carbonate, etc.). In some embodiments, the base is an alkali metal hydroxide (e.g., sodium hydroxide, potassium hydroxide, lithium hydroxide, etc.). In some embodiments, the base is potassium carbonate. In some embodiments, the base is an aqueous solution of an inorganic base. In some embodiments, the base is an aqueous solution of an alkali metal carbonate. In some embodiments, the base is an aqueous solution of potassium carbonate.

In some embodiments, step (b) further comprises step (b3): separating the step (b2) mixture to obtain an organic phase. The separating of step (b3) can comprise allowing the step (b2) mixture to partition into an aqueous phase and an organic phase, and removing the aqueous phase.

In some embodiments, step (b) further comprises step (b4): concentrating the organic phase to a first concentrated solution having less than about 5.0 wt % water. In some embodiments, the first concentrated solution has less than about 2.0 wt % water. In some embodiments, the first concentrated solution has about 5.0 wt % to about 0.001 wt % water. In some embodiments, the first concentrated solution has about 1.0 wt % to about 0.001 wt % water. In some embodiments, the first concentrated solution has about 1.0 wt % to about 0.01 wt % water. In some embodiments, the first concentrated solution has about 0.5 wt % to about 0.01 wt % water. The water content of the first concentrated solution can be determined by, e.g., Karl Fischer titration.

In some embodiments, step (b) further comprises step (b5): concentrating the first concentrated solution to a second concentrated solution, wherein the second concentrated solution has a total weight that is about 15 wt % to about 65 wt % of the first concentrated solution. In some embodiments, the second concentrated solution has a total weight that is about 35 wt % to about 40 wt % of the first concentrated solution. In some embodiments, the second concentrated solution has a total weight that is about 33 wt % to about 38 wt % of the first concentrated solution. In some embodiments, the second concentrated solution has a total weight that is about 35 wt % of the first concentrated solution.

In some embodiments, step (b) can further comprise step (b5): concentrating the first concentrated solution to a second concentrated solution, wherein the second concentrated solution has a total weight of (R)-(+)-amisulpride or (S)-(−)-amisulpride that is about 15 wt % to about 65 wt % of the second concentrated solution. In some embodiments, the second concentrated solution has a total weight of (R)-(+)-amisulpride or (S)-(−)-amisulpride that is about 35 wt % to about 45 wt % of the second concentrated solution. In some embodiments, the second concentrated solution has a total weight of (R)-(+)-amisulpride or (S)-(−)-amisulpride that is about 33 wt % to about 38 wt % of the second concentrated solution. In some embodiments, the second concentrated solution has a total weight of (R)-(+)-amisulpride or (S)-(−)-amisulpride that is about 35 wt % to about 40 wt % of the second concentrated solution. In some embodiments, the second concentrated solution has a total weight of (R)-(+)-amisulpride or (S)-(−)-amisulpride that is about 35 wt % of the second concentrated solution.

In some embodiments, step (b5) can further comprise adding S5, wherein S5 is a solvent, to the second concentrated solution. In some embodiments, S5 is an ether solvent. In some embodiments, S5 is methyl tert-butyl ether.

In some embodiments, step (b) can further comprise step (b6): adding a seed amount of a crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2° to the second concentrated solution to form a seeded mixture.

Alternatively, In some embodiments, step (b) can further comprise step (b6): adding a seed amount of a crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2° to the second concentrated solution to form a seeded mixture.

In some embodiments of step (b6), the seed amount has a total weight that is about 0.001 wt % to about 15 wt % of the second concentrated solution. In some embodiments, the seed amount has a total weight that is about 0.1 wt % to about 10 wt % of the second concentrated solution. In some embodiments, the seed amount has a total weight that is about 0.1 wt % to about 5 wt % of the second concentrated solution. In some embodiments, the seed amount has a total weight that is about 0.1 wt % to about 2.0 wt % of the second concentrated solution. In some embodiments, the seed amount has a total weight that is about 0.4 wt %, about 1.4 wt %, or about 2.0 wt % of the second concentrated solution. In some embodiments, the seed amount has a total weight that is about 0.4 wt % of the second concentrated solution. In some embodiments, the seed amount has a total weight that is about 1.4 wt % of the second concentrated solution. In some embodiments, the seed amount has a total weight that is about 2.0 wt % of the second concentrated solution.

In some embodiments of step (b6), the seed amount has a total weight that is about 0.001 wt % to about 15 wt % of the expected yield of the (R)- or (S)-amisulpride present in the second concentrated solution. In some embodiments, the seed amount has a total weight that is about 0.1 wt % to about 10 wt % of the expected yield of the (R)- or (S)-amisulpride present in the second concentrated solution. In some embodiments, the seed amount has a total weight that is about 0.1 wt % to about 5 wt % of the expected yield of the (R)- or (S)-amisulpride present in the second concentrated solution. In some embodiments, the seed amount has a total weight that is about 0.1 wt % to about 2.0 wt % of the expected yield of the (R)- or (S)-amisulpride present in the second concentrated solution. In some embodiments, the seed amount has a total weight that is about 0.1 wt % to about 1.0 wt % of the expected yield of the (R)- or (S)-amisulpride present in the second concentrated solution. In some embodiments, the seed amount has a total weight that is about 0.4 wt %, about 1.4 wt %, or about 2.0 wt % of the expected yield of the (R)- or (S)-amisulpride present in the second concentrated solution. In some embodiments, the seed amount has a total weight that is about 0.4 wt % of the expected yield of the (R)- or (S)-amisulpride present in the second concentrated solution. In some embodiments, the seed amount has a total weight that is about 1.4 wt % of the expected yield of the (R)- or (S)-amisulpride present in the second concentrated solution. In some embodiments, the seed amount has a total weight that is about 2.0 wt % of the expected yield of the (R)- or (S)-amisulpride present in the second concentrated solution. In some embodiments, the seed amount has a total weight that is about 0.75 wt % of the expected yield of the (R)- or (S)-amisulpride present in the second concentrated solution.

In some embodiments, step (b) further comprises step (b7): filtering the seeded mixture of step (b6) to obtain a product solid.

In some embodiments, step (b) further comprises step (b8): drying the product solid to obtain a crude product.

In some embodiments, step (b) further comprises step (b9): recrystallizing the crude product in the presence of the isolation reagent. In some embodiments, the recrystallizing comprises (i) dissolving the crude product with the isolation reagent to form a recrystallization solution, (ii) filtering and concentrating the recrystallization solution, and (iii) adding a seed amount of a crystalline form of (R)-(+)-amisulpride or a crystalline form of (S)-(−)-amisulpride. In some embodiments, the dissolving comprises heating the recrystallization solution to a temperature between about 40° C. and about 70° C., or between about 45° C. and about 55° C., or about 50° C.

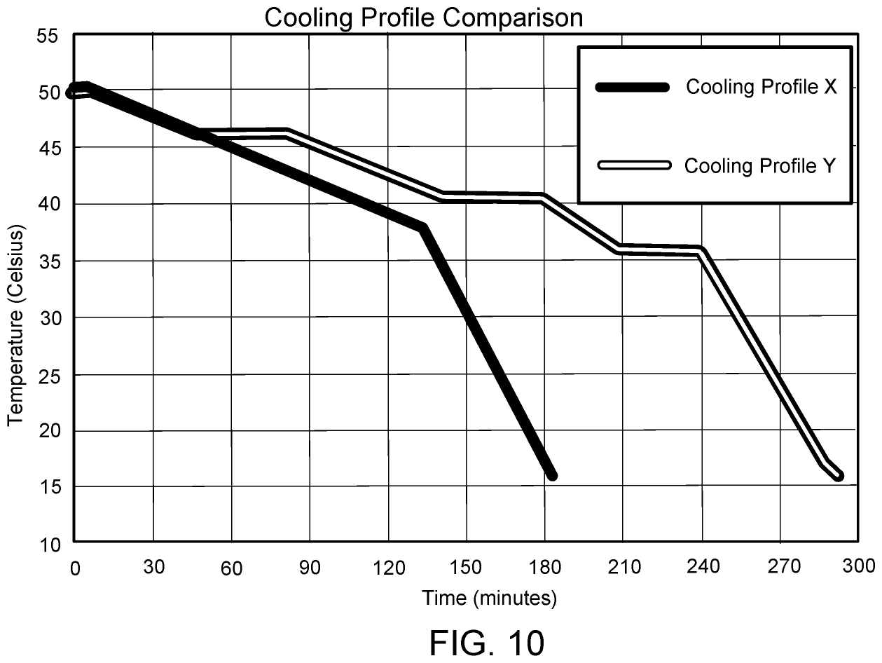

In some embodiments, step (b) further comprises step (b9): recrystallizing the crude product in the presence of the isolation reagent. In some embodiments, the recrystallizing comprises (i) heating the crude product in the presence of an isolation reagent at a first elevated temperature to form a recrystallization solution, (ii) filtering and concentrating the recrystallization solution to form a concentrated recrystallization solution; (iii) adding S5, wherein S5 is a solvent, to the concentrated recrystallization solution; (iv) adding a seed amount of a crystalline form of (R)-(+)-amisulpride or a crystalline form of (S)-(−)-amisulpride to the concentrated recrystallization solution of step (iii) to form a seeded recrystallization solution; and (v) cooling the seeded recrystallization solution. In some embodiments, the first elevated temperature is from about 45° C. to about 60° C. In some embodiments, the first elevated temperature is from about 50° C. to about 55° C. In some embodiments, the cooling comprises cooling to a first reduced temperature over a first period of time, and then cooling to a second reduced temperature over a second period of time. In some embodiments, the cooling corresponds substantially to the cooling profile shown for the plot of Example 16 in FIG. 10. In some embodiments, the first reduced temperature is from about 38° C. to about 42° C. In some embodiments, the first period of time is about 120 min. In some embodiments, the first period of time is from about 90 min to about 150 min. In some embodiments, the second reduced temperature is from about 10° C. to about 15° C. In some embodiments, the second period of time is about 60 min. In some embodiments, the second period of time is about 30 min to about 90 min. In some embodiments, the cooling over the first period of time is performed at a first cooling rate. In some embodiments, the cooling over the second period of time is performed at a second cooling rate. In some embodiments, the second cooling rate is greater than the first cooling rate. In some embodiments, the first cooling rate is about 0.1° C./min. In some embodiments, the second cooling rate is about 0.5° C./min. In some embodiments, S5 is an ether solvent. In some embodiments, S5 is methyl tert-butyl ether. The recrystallization procedures using the cooling profile of Example 16 can provide for the formation of crystalline solids having narrower and smaller particle size distribution (PSD) compared to other crystallization procedures (see, e.g., Example 16).

In some embodiments, the tertiary amine is of the formula

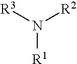

wherein:
(i) R¹, R² and R³ are each independently C₁₋₆ alkyl, C₃₋₆ cycloalkyl, 3-10 membered monocyclic or bicyclic heterocycloalkyl, or 5-10 membered monocyclic heteroaryl; or
(ii) R¹ is C₁₋₆ alkyl, C₃₋₆ cycloalkyl, 3-10 membered monocyclic or bicyclic heterocycloalkyl, or 5-10 membered monocyclic heteroaryl, and R² and R³ together with the N atom to which they are attached form a 3-10 membered monocyclic or bicyclic heterocycloalkyl or a 5-10 membered monocyclic heteroaryl.

In some embodiments, R¹, R² and R³ are each independently C₁₋₆ alkyl, C₃₋₆ cycloalkyl, 3-10 membered monocyclic or bicyclic heterocycloalkyl, or 5-10 membered monocyclic heteroaryl. In some embodiments, the tertiary amine is triethyl amine.

In some embodiments, R² and R³ together with the N atom to which they are attached form a 3-10 membered monocyclic or bicyclic heterocycloalkyl or a 5-10 membered monocyclic heteroaryl. In some embodiments, the tertiary amine is 4-methylmorpholine.

In some embodiments, the acid activating reagent is of the formula

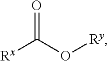

wherein R^{x} is halogen and R^{y} is C₁₋₅ alkyl. In some embodiments, R^{x} is chloro. In some embodiments, R^{y} is ethyl. In some embodiments, the acid activating reagent is an alkyl chloroformate. In some embodiments, the acid activating reagent is methyl chloroformate or ethyl chloroformate. In some embodiments, the acid activating reagent is ethyl chloroformate.

As used herein, the term “isolation reagent” refers to a compound that facilitates the precipitation of crystalline products but does not participate in a chemical transformation. In some embodiments, the isolation reagent is of the formula

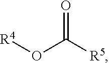

wherein R⁴ is C₁₋₆ alkyl; and R⁵ is C₁₋₅ alkyl or C₁₋₅ alkoxide. In some embodiments, the isolation reagent is of the formula

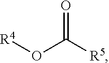

wherein R⁴ is C₃₋₆ alkyl; and R⁵ is C₁₋₅ alkyl. In some embodiments, the isolation reagent is not ethyl acetate. In some embodiments, the isolation reagent is isopropyl acetate, n-propyl acetate, t-butyl acetate, isobutyl acetate, or n-butyl acetate. In some embodiments, the isolation reagent is isopropyl acetate.

Unless otherwise specified, alkyl (or alkylene) is intended to include linear or branched saturated hydrocarbon structures and combinations thereof. Alkyl refers to alkyl groups from 1 to 20 carbon atoms. Examples of alkyl groups include methyl, ethyl, propyl, isopropyl, n-butyl, s-butyl, t-butyl and the like. Furthermore, as used herein, the term “Cᵢ₋ⱼ,” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

Unless otherwise specified, “alkoxide” refers to an —O—C₁₋₂₀ alkyl group. Alkoxide is intended to include linear or branched structures and combinations thereof. Examples of alkyl groups include methoxy, ethoxy, isopropoxy, and the like.

In some embodiments, the isolation reagent is of the formula

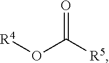

wherein R⁴ is C₁₋₆ alkyl; and R⁵ is C₁₋₅ alkoxide. In some embodiments, the isolation reagent is diethyl carbonate or dimethyl carbonate.

In some embodiments, the isolation reagent is of the formula R⁶COR⁷, where each of R⁶ and R⁷ is independently C₁₋₅ alkyl. In some embodiments, the isolation reagent is 2-butanone or 3-pentanone. In some embodiments, the isolation reagent is 3-pentanone.

In some embodiments, the method can further comprise recrystallizing the enantiomerically pure crystalline form of (R)-(+)-amisulpride or the enantiomerically pure crystalline form of (S)-(−)-amisulpride in the presence of isopropyl acetate.

The seed amount of a crystalline form of (R)-(+)-amisulpride can have a greater than about 95% chemical purity and a greater than about 95% enantiomeric purity. In some embodiments, the seed amount of a crystalline form of (R)-(+)-amisulpride has a greater than about 98% chemical purity and a greater than about 98% enantiomeric purity. In some embodiments, the enantiomerically pure crystalline form of (R)-(+)-amisulpride has a greater than about 95% chemical purity. In some embodiments, the enantiomerically pure crystalline form of (R)-(+)-amisulpride has a greater than about 98% chemical purity. In some embodiments, the enantiomerically pure crystalline form of (R)-(+)-amisulpride has a greater than about 99% chemical purity. In some embodiments, the enantiomerically pure crystalline form of (R)-(+)-amisulpride has a greater than about 95% enantiomeric purity. In some embodiments, the enantiomerically pure crystalline form of (R)-(+)-amisulpride has a greater than about 98% enantiomeric purity. In some embodiments, the enantiomerically pure crystalline form of (R)-(+)-amisulpride has a greater than about 99% enantiomeric purity.

The seed amount of a crystalline form of (S)-(−)-amisulpride can have a greater than about 95% chemical purity and a greater than about 95% enantiomeric purity. In some embodiments, the seed amount of a crystalline form of (S)-(−)-amisulpride has a greater than about 98% chemical purity and a greater than about 98% enantiomeric purity. In some embodiments, the enantiomerically pure crystalline form of (S)-(−)-amisulpride has a greater than about 95% chemical purity. In some embodiments, the enantiomerically pure crystalline form of (S)-(−)-amisulpride has a greater than about 98% chemical purity. In some embodiments, the enantiomerically pure crystalline form of (S)-(−)-amisulpride has a greater than about 99% chemical purity. In some embodiments, the enantiomerically pure crystalline form of (S)-(−)-amisulpride has a greater than about 95% enantiomeric purity. In some embodiments, the enantiomerically pure crystalline form of (S)-(−)-amisulpride has a greater than about 98% enantiomeric purity. In some embodiments, wherein the enantiomerically pure crystalline form of (S)-(−)-amisulpride has a greater than about 99% enantiomeric purity.

Also provided herein is a method of preparing an enantiomerically pure crystalline form of (R)-(+)-amisulpride comprising the steps of:
(a1) reacting 4-amino-5-(ethylsulfonyl)-2-methoxybenzoic acid with the tertiary amine and the acid activating reagent to form a first reaction mixture;
(a2) adding (R)-(1-ethylpyrrolidin-2-yl)methanamine to the first reaction mixture;
(b) isolating from the reaction mixture of step (a2), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°; wherein step (b) does not comprise formation of a solvate of (R)-(+)-amisulpride.

Also provided herein is a method of preparing an enantiomerically pure crystalline form of (R)-(+)-amisulpride consisting essentially of the following steps:
(a1) reacting 4-amino-5-(ethylsulfonyl)-2-methoxybenzoic acid with the tertiary amine and the acid activating reagent to form a first reaction mixture;
(a2) adding (R)-(1-ethylpyrrolidin-2-yl)methanamine to the first reaction mixture;
(b) isolating from the reaction mixture of step (a2), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°; wherein step (b) does not comprise formation of a solvate of (R)-(+)-amisulpride.

Also provided herein is a method of preparing an enantiomerically pure crystalline form of (R)-(+)-amisulpride consisting of the following steps:
(a1) reacting 4-amino-5-(ethylsulfonyl)-2-methoxybenzoic acid with the tertiary amine and the acid activating reagent to form a first reaction mixture;
(a2) adding (R)-(1-ethylpyrrolidin-2-yl)methanamine to the first reaction mixture;
(b) isolating from the reaction mixture of step (a2), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°; wherein step (b) does not comprise formation of a solvate of (R)-(+)-amisulpride.

Also provided herein is a method of preparing an enantiomerically pure crystalline form of (S)-(−)-amisulpride comprising the steps of:
(a1) reacting 4-amino-5-(ethylsulfonyl)-2-methoxybenzoic acid with the tertiary amine and the acid activating reagent to form a first reaction mixture;
(a2) adding (S)-(1-ethylpyrrolidin-2-yl)methanamine to the first reaction mixture;
(b) isolating from the reaction mixture of step (a2), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°; wherein step (b) does not comprise formation of a solvate of (S)-(−)-amisulpride.

Also provided herein is a method of preparing an enantiomerically pure crystalline form of (S)-(−)-amisulpride consisting essentially of the following steps:
(a1) reacting 4-amino-5-(ethylsulfonyl)-2-methoxybenzoic acid with the tertiary amine and the acid activating reagent to form a first reaction mixture;
(a2) adding (S)-(1-ethylpyrrolidin-2-yl)methanamine to the first reaction mixture;
(b) isolating from the reaction mixture of step (a2), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°; wherein step (b) does not comprise formation of a solvate of (S)-(−)-amisulpride.

Also provided herein is a method of preparing an enantiomerically pure crystalline form of (S)-(−)-amisulpride consisting of the following steps:
(a1) reacting 4-amino-5-(ethylsulfonyl)-2-methoxybenzoic acid with the tertiary amine and the acid activating reagent to form a first reaction mixture;
(a2) adding (S)-(1-ethylpyrrolidin-2-yl)methanamine to the first reaction mixture;
(b) isolating from the reaction mixture of step (a2), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°; wherein step (b) does not comprise formation of a solvate of (S)-(−)-amisulpride.

### Salt Methods

Provided herein is a method of preparing an enantiomerically pure crystalline form of (R)-(+)-amisulpride comprising the steps of.
(a) reacting 4-amino-5-(ethylsulfonyl)-2-methoxybenzoic acid with a tertiary amine and an acid activating reagent to form a reaction mixture;
(b) adding a (R)-(1-ethylpyrrolidin-2-yl)methanamine salt to the reaction mixture to form a mixture of step (b); and
(c) isolating from the mixture of step (b), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2°.

In some embodiments, step (c) does not comprise formation of a solvate of (R)-(+)-amisulpride. In some embodiments, step (c) does not comprise formation of an ethyl acetate solvate of (R)-(+)-amisulpride.

In some embodiments, the isolating of step (c) can further comprise:
(d1) adding the isolation reagent to the mixture of step (b), followed by an aqueous base solution to form a mixture of step (d1),
(d2) separating the mixture of step (d1) to obtain an organic phase;
(d3) concentrating the organic phase of step (d2) to produce a product solution having about 5.0 wt % to about 0.001 wt % water;
(d4) adding to the product solution a seed amount of a crystalline form of (R)-(+)-amisulpride, characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (R)-(+)-amisulpride.

In some embodiments, the method further comprises recrystallizing the enantiomerically pure crystalline form of (R)-(+)-amisulpride of step (d6). In some embodiments, the recrystallizing comprises (i) heating the enantiomerically pure crystalline form of (R)-(+)-amisulpride of step (d6) in the presence of an isolation reagent at a first elevated temperature to form a recrystallization solution, (ii) filtering and concentrating the recrystallization solution to form a concentrated recrystallization solution; (iii) adding S5, wherein S5 is a solvent, to the concentrated recrystallization solution; (iv) adding a seed amount of a crystalline form of (R)-(+)-amisulpride to the concentrated recrystallization solution of step (iii) to form a seeded recrystallization solution; and (v) cooling the seeded recrystallization solution. In some embodiments, the cooling comprises cooling to a first reduced temperature over a first period of time, and then cooling to a second reduced temperature over a second period of time. In some embodiments, the first elevated temperature is from about 45° C. to about 60° C. In some embodiments, the first elevated temperature is from about 50° C. to about 55° C. In some embodiments, the cooling corresponds substantially to the cooling profile shown for the plot of Example 16 in FIG. 10. In some embodiments, the first reduced temperature is from about 38° C. to about 42° C. In some embodiments, the first period of time is about 120 min. In some embodiments, the first period of time is from about 90 min to about 150 min. In some embodiments, the second reduced temperature is from about 10° C. to about 15° C. In some embodiments, the second period of time is about 60 min. In some embodiments, the second period of time is about 30 min to about 90 min. In some embodiments, the cooling over the first period of time is performed at a first cooling rate. In some embodiments, the cooling over the second period of time is performed at a second cooling rate. In some embodiments, the second cooling rate is greater than the first cooling rate. In some embodiments, the first cooling rate is about 0.1° C./min. In some embodiments, the second cooling rate is about 0.5° C./min. In some embodiments, S5 is an ether solvent. In some embodiments, S5 is methyl tert-butyl ether. The recrystallization procedures using the cooling profile of Example 16 can provide for the formation of crystalline solids having narrower and smaller particle size distribution (PSD) compared to other crystallization procedures (see, e.g., Example 16).

Also provided herein is a method of preparing an enantiomerically pure crystalline form of (S)-(−)-amisulpride comprising the steps of:
(a) reacting 4-amino-5-(ethylsulfonyl)-2-methoxybenzoic acid with a tertiary amine and an acid activating reagent to form a reaction mixture;
(b) adding a (S)-(1-ethylpyrrolidin-2-yl)methanamine salt to the reaction mixture of step (a) to form a mixture of step (b); and
(c) isolating from the mixture of step (b), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ), at least at 7.0±0.2° and 9.7±0.2°, and further peaks at 15.4±0.2° and/or 19.4±0.2°.

In some embodiments, step (c) does not comprise formation of a solvate of (S)-(−)-amisulpride. In some embodiments, step (c) does not comprise formation of an ethyl acetate solvate of (S)-(−)-amisulpride.

In some embodiments, the isolating of step (c) can comprise:
(d1) adding the isolation reagent to the mixture of step (b), followed by an aqueous base solution to form a mixture of step (d1);
(d2) separating the mixture of step (d1) to obtain an organic phase;
(d3) concentrating the organic phase of step (d2) to produce a product solution with about 5.0 wt % to about 0.01 wt % water;
(d4) adding to the product solution of step (d3) with a seed amount of a crystalline form of (S)-(−)-amisulpride characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (S)-(−)-amisulpride.

In some embodiments, the method further comprises recrystallizing the enantiomerically pure crystalline form of (S)-(−)-amisulpride of step (d6). In some embodiments, the recrystallizing comprises (i) heating the enantiomerically pure crystalline form of (S)-(−)-amisulpride of step (d6) in the presence of an isolation reagent at a first elevated temperature to form a recrystallization solution, (ii) filtering and concentrating the recrystallization solution to form a concentrated recrystallization solution; (iii) adding S5, wherein S5 is a solvent, to the concentrated recrystallization solution; (iv) adding a seed amount of a crystalline form of (S)-(−)-amisulpride to the concentrated recrystallization solution of step (iii) to form a seeded recrystallization solution; and (v) cooling the seeded recrystallization solution. In some embodiments, the first elevated temperature is from about 45° C. to about 60° C. In some embodiments, the first elevated temperature is from about 50° C. to about 55° C. In some embodiments, the cooling comprises cooling to a first reduced temperature over a first period of time, and then cooling to a second reduced temperature over a second period of time. In some embodiments, the cooling corresponds substantially to the cooling profile shown for the plot of Example 16 in FIG. 10. In some embodiments, the first reduced temperature is from about 38° C. to about 42° C. In some embodiments, the first period of time is about 120 min. In some embodiments, the first period of time is from about 90 min to about 150 min. In some embodiments, the second reduced temperature is from about 10° C. to about 15° C. In some embodiments, the second period of time is about 60 min. In some embodiments, the second period of time is about 30 min to about 90 min. In some embodiments, the cooling over the first period of time is performed at a first cooling rate. In some embodiments, the cooling over the second period of time is performed at a second cooling rate. In some embodiments, the second cooling rate is greater than the first cooling rate. In some embodiments, the first cooling rate is about 0.1° C./min. In some embodiments, the second cooling rate is about 0.5° C./min. In some embodiments, S5 is an ether solvent. In some embodiments, S5 is methyl tert-butyl ether.

In some embodiments, the reacting of step (a) is performed in the presence of S3, wherein S3 is a solvent. In some embodiments S3 is a polar aprotic solvent. In some embodiments, S3 is acetone.

In some embodiments, step (b) further comprises adding an additional amount of the tertiary amine of step (a) to the mixture of step (b).

In some embodiments, the product solution of step (d3) has about 0.001 wt % to about 0.5 wt % water. In some embodiments, the product solution of step (d3) has about 0.001 wt % to about 0.2 wt % water. In some embodiments, the product solution of step (d3) has about 0.001 wt % to about 0.1 wt % water. In some embodiments, the product solution of step (d3) has about 0.01 wt % to about 0.2 wt % water.

In some embodiments, the tertiary amine is of the formula

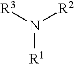

wherein:
(i) R¹, R² and R³ are each independently C₁₋₆ alkyl, C₃₋₆ cycloalkyl, 3-10 membered monocyclic or bicyclic heterocycloalkyl, or 5-10 membered monocyclic heteroaryl; or
(ii) R¹ is C₁₋₆ alkyl, C₃₋₆ cycloalkyl, 3-10 membered monocyclic or bicyclic heterocycloalkyl, or 5-10 membered monocyclic heteroaryl, and R² and R³ together with the N atom to which they are attached form a 3-10 membered monocyclic or bicyclic heterocycloalkyl or a 5-10 membered monocyclic heteroaryl.

In some embodiments, R¹, R² and R³ are each independently C₁₋₆ alkyl, C₃₋₆ cycloalkyl, 3-10 membered monocyclic or bicyclic heterocycloalkyl, or 5-10 membered monocyclic heteroaryl. In some embodiments, the tertiary amine is triethyl amine.

In some embodiments, R² and R³ together with the N atom to which they are attached form a 3-10 membered monocyclic or bicyclic heterocycloalkyl or a 5-10 membered monocyclic heteroaryl. In some embodiments, the tertiary amine is 4-methylmorpholine.

In some embodiments, the acid activating reagent is of the formula

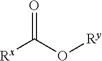

wherein R^{x} is halogen and R^{y} is C₁₋₅ alkyl. In some embodiments, R^{x} is chloro. In some embodiments, R^{y} is ethyl. In some embodiments, the acid activating reagent is an alkyl chloroformate. In some embodiments, the acid activating reagent is methyl chloroformate or ethyl chloroformate. In some embodiments, the acid activating reagent is ethyl chloroformate.

In some embodiments, the isolation reagent is of the formula

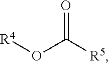

wherein R⁴ is C₁₋₆ alkyl; and R⁵ is C₁₋₅ alkyl or C₁₋₅ alkoxide. In some embodiments, the isolation reagent is of the formula

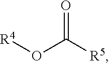

wherein R⁴ is C₃₋₆ alkyl; and R⁵ is C₁₋₅ alkyl. In some embodiments, the isolation reagent is not ethyl acetate. In some embodiments, the isolation reagent is isopropyl acetate, methyl acetate, n-propyl acetate, t-butyl acetate, isobutyl acetate, or n-butyl acetate. In some embodiments, the isolation reagent is isopropyl acetate.

In some embodiments, the isolation reagent is of the formula

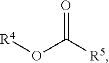

wherein R⁴ is C₁₋₆ alkyl; and R⁵ is C₁₋₅ alkoxide. In some embodiments, the isolation reagent is diethyl carbonate or dimethyl carbonate.

In some embodiments, the isolation reagent is of the formula R⁶COR⁷, where each of R⁶ and R⁷ is independently C₁₋₅ alkyl. In some embodiments, the isolation reagent is 2-butanone or 3-pentanone. In some embodiments, the isolation reagent is 3-pentanone.

In some embodiments, the (R)-(1-ethylpyrrolidin-2-yl)methanamine salt is a bis tartrate salt of (R)-(1-ethylpyrrolidin-2-yl)methanamine. In some embodiments, the (R)-(1-ethylpyrrolidin-2-yl)methanamine salt is a bis L-tartrate salt of (R)-(1-ethylpyrrolidin-2-yl)methanamine.

In some embodiments, the (S)-(1-ethylpyrrolidin-2-yl)methanamine salt is a bis tartrate salt of (S)-(1-ethylpyrrolidin-2-yl)methanamine. In some embodiments, the (S)-(1-ethylpyrrolidin-2-yl)methanamine salt is a bis D-tartrate salt of (S)-(1-ethylpyrrolidin-2-yl)methanamine.

The method can further comprise recrystallizing the enantiomerically pure crystalline form of (R)-(+)-amisulpride or the enantiomerically pure crystalline form of (S)-(−)-amisulpride in the presence of isopropyl acetate. The method can further comprise recrystallizing the enantiomerically pure crystalline form of (R)-(+)-amisulpride in the presence of an isolation reagent. The method can further comprise recrystallizing the enantiomerically pure crystalline form of (S)-(−)-amisulpride in the presence of an isolation reagent. In some embodiments, the isolation reagent is isopropyl acetate.

The seed amount of a crystalline form of (R)-(+)-amisulpride can have a greater than about 95% chemical purity and a greater than about 95% enantiomeric purity. The seed amount of a crystalline form of (R)-(+)-amisulpride can have a greater than about 98% chemical purity and a greater than about 98% enantiomeric purity.

In some embodiments, the enantiomerically pure crystalline form of (R)-(+)-amisulpride has a greater than about 95% chemical purity. In some embodiments, the enantiomerically pure crystalline form of (R)-(+)-amisulpride has a greater than about 98% chemical purity. In some embodiments, the enantiomerically pure crystalline form of (R)-(+)-amisulpride has a greater than about 99% chemical purity. In some embodiments, the enantiomerically pure crystalline form of (R)-(+)-amisulpride has a greater than about 95% enantiomeric purity. In some embodiments, the enantiomerically pure crystalline form of (R)-(+)-amisulpride has a greater than about 98% enantiomeric purity. In some embodiments, the enantiomerically pure crystalline form of (R)-(+)-amisulpride has a greater than about 99% enantiomeric purity.

The method can further comprise recrystallizing the enantiomerically pure crystalline form of (R)-(+)-amisulpride in the presence of an isolation reagent. In some embodiments, the isolation reagent is isopropyl acetate. In some embodiments, the recrystallizing comprises dissolving (R)-(+)-amisulpride in isopropyl acetate to form a solution of (R)-(+)-amisulpride in isopropyl acetate, adding a seed amount of (R)-(+)-amisulpride to the solution of (R)-(+)-amisulpride in isopropyl acetate, and cooling the solution of (R)-(+)-amisulpride in isopropyl acetate to precipitate the crystalline form of (R)-(+)-amisulpride. In some embodiments, the dissolving comprises heating the solution to a temperature between about 40° C. and about 70° C., or between about 45° C. and about 55° C., or about 50° C.

The seed amount of a crystalline form of (S)-(−)-amisulpride can have a greater than about 95% chemical purity and a greater than about 95% enantiomeric purity. The seed amount of a crystalline form of (S)-(−)-amisulpride can have a greater than about 98% chemical purity and a greater than about 98% enantiomeric purity.

In some embodiments, the enantiomerically pure crystalline form of (S)-(−)-amisulpride has a greater than about 95% chemical purity. In some embodiments, the enantiomerically pure crystalline form of (S)-(−)-amisulpride has a greater than about 98% chemical purity. In some embodiments, the enantiomerically pure crystalline form of (S)-(−)-amisulpride has a greater than about 99% chemical purity. In some embodiments, the enantiomerically pure crystalline form of (S)-(−)-amisulpride has a greater than about 95% enantiomeric purity. In some embodiments, the enantiomerically pure crystalline form of (S)-(−)-amisulpride has a greater than about 98% enantiomeric purity. In some embodiments, wherein the enantiomerically pure crystalline form of (S)-(−)-amisulpride has a greater than about 99% enantiomeric purity.

Provided herein is a method of preparing an enantiomerically pure crystalline form of (R)-(+)-amisulpride comprising one or more steps selected from:
(a) reacting 4-amino-5-(ethylsulfonyl)-2-methoxybenzoic acid with a tertiary amine and an acid activating reagent to form a reaction mixture;
(b) adding a (R)-(1-ethylpyrrolidin-2-yl)methanamine salt to the reaction mixture to form a mixture of step (b);
(d1) adding an isolation reagent to the mixture of step (b), followed by an aqueous base solution to form a mixture of step (d1);
(d2) separating the mixture of step (d1) to obtain an organic phase;
(d3) concentrating the organic phase of step (d2) to produce a product solution having about 5.0 wt % to about 0.001 wt % water;
(d4) adding to the product solution a seed amount of a crystalline form of (R)-(+)-amisulpride, characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (R)-(+)-amisulpride.

Provided herein is a method of preparing an enantiomerically pure crystalline form of (R)-(+)-amisulpride consisting essentially of the following steps:
(a) reacting 4-amino-5-(ethylsulfonyl)-2-methoxybenzoic acid with a tertiary amine and an acid activating reagent to form a reaction mixture;
(b) adding a (R)-(1-ethylpyrrolidin-2-yl)methanamine salt to the reaction mixture to form a mixture of step (b);
(d1) adding an isolation reagent to the mixture of step (b), followed by an aqueous base solution to form a mixture of step (d1);
(d2) separating the mixture of step (d1) to obtain an organic phase;
(d3) concentrating the organic phase of step (d2) to produce a product solution having about 5.0 wt % to about 0.001 wt % water;
(d4) adding to the product solution a seed amount of a crystalline form of (R)-(+)-amisulpride, characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (R)-(+)-amisulpride.

Provided herein is a method of preparing an enantiomerically pure crystalline form of (R)-(+)-amisulpride consisting of the following steps:
(a) reacting 4-amino-5-(ethylsulfonyl)-2-methoxybenzoic acid with a tertiary amine and an acid activating reagent to form a reaction mixture;
(b) adding a (R)-(1-ethylpyrrolidin-2-yl)methanamine salt to the reaction mixture to form a mixture of step (b);
(d1) adding an isolation reagent to the mixture of step (b), followed by an aqueous base solution to form a mixture of step (d1);
(d2) separating the mixture of step (d1) to obtain an organic phase;
(d3) concentrating the organic phase of step (d2) to produce a product solution having about 5.0 wt % to about 0.001 wt % water;
(d4) adding to the product solution a seed amount of a crystalline form of (R)-(+)-amisulpride, characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (R)-(+)-amisulpride.

Provided herein is a method of preparing an enantiomerically pure crystalline form of (S)-(−)-amisulpride comprising one or more steps selected from:
(a) reacting 4-amino-5-(ethylsulfonyl)-2-methoxybenzoic acid with a tertiary amine and an acid activating reagent to form a reaction mixture;
(b) adding a (S)-(1-ethylpyrrolidin-2-yl)methanamine salt to the reaction mixture to form a mixture of step (b);
(d1) adding an isolation reagent to the mixture of step (b), followed by an aqueous base solution to form a mixture of step (d1);
(d2) separating the mixture of step (d1) to obtain an organic phase;
(d3) concentrating the organic phase of step (d2) to produce a product solution having about 5.0 wt % to about 0.001 wt % water;
(d4) adding to the product solution a seed amount of a crystalline form of (S)-(−)-amisulpride, characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (S)-(−)-amisulpride.

Provided herein is a method of preparing an enantiomerically pure crystalline form of (S)-(−)-amisulpride consisting essentially of the following steps:
(a) reacting 4-amino-5-(ethylsulfonyl)-2-methoxybenzoic acid with a tertiary amine and an acid activating reagent to form a reaction mixture;
(b) adding a (S)-(1-ethylpyrrolidin-2-yl)methanamine salt to the reaction mixture to form a mixture of step (b);
(d1) adding an isolation reagent to the mixture of step (b), followed by an aqueous base solution to form a mixture of step (d1);
(d2) separating the mixture of step (d1) to obtain an organic phase;
(d3) concentrating the organic phase of step (d2) to produce a product solution having about 5.0 wt % to about 0.001 wt % water;
(d4) adding to the product solution a seed amount of a crystalline form of (S)-(−)-amisulpride, characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (S)-(−)-amisulpride.

Provided herein is a method of preparing an enantiomerically pure crystalline form of (S)-(−)-amisulpride consisting of the following steps:
(a) reacting 4-amino-5-(ethylsulfonyl)-2-methoxybenzoic acid with a tertiary amine and an acid activating reagent to form a reaction mixture;
(b) adding a (S)-(1-ethylpyrrolidin-2-yl)methanamine salt to the reaction mixture to form a mixture of step (b);
(d1) adding an isolation reagent to the mixture of step (b), followed by an aqueous base solution to form a mixture of step (d1);
(d2) separating the mixture of step (d1) to obtain an organic phase;
(d3) concentrating the organic phase of step (d2) to produce a product solution having about 5.0 wt % to about 0.001 wt % water;
(d4) adding to the product solution a seed amount of a crystalline form of (S)-(−)-amisulpride, characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (S)-(−)-amisulpride.

### Recrystallization Methods

Provided herein is a method of preparing an enantiomerically pure crystalline form of (R)-(+)-amisulpride comprising the steps of:
(a) heating (R)-(+)-amisulpride (e.g., crude, amorphous, or Form A of (R)-(+)-amisulpride) in the presence of an isolation reagent to a first elevated temperature to form a crystallization mixture;
(b) adding a seed amount of Form A of (R)-(+)-amisulpride to the crystallization mixture to form a seeded mixture;
(c) cooling the seeded mixture to a first reduced temperature over a first period of time;
(d) cooling the seeded mixture from step (c) to a second reduced temperature over a second period of time; and
(e) filtering the seeded mixture from step (d) to provide an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°.

In some embodiments, the (R)-(+)-amisulpride of step (a) is crude (R)-(+)-amisulpride. In some embodiments, the (R)-(+)-amisulpride of step (a) is amorphous (R)-(+)-amisulpride. In some embodiments, the (R)-(+)-amisulpride of step (a) is Form A of (R)-(+)-amisulpride.

Also provided herein is a method of preparing an enantiomerically pure crystalline form of (S)-(−)-amisulpride comprising the steps of:
(a) heating (S)-(−)-amisulpride (e.g., crude, amorphous, or Form A′ of (S)-(−)-amisulpride) in the presence of an isolation reagent to a first elevated temperature to form a crystallization mixture;
(b) adding a seed amount of Form A′ of (S)-(−)-amisulpride to the crystallization mixture to form a seeded mixture;
(c) cooling the seeded mixture to a first reduced temperature over a first period of time;
(d) cooling the seeded mixture of step (c) to a second reduced temperature over a second period of time; and
(e) filtering the seeded mixture of step (d) to provide an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°.

In some embodiments, the (S)-(−)-amisulpride of step (a) is crude (S)-(−)-amisulpride. In some embodiments, the (S)-(−)-amisulpride of step (a) is amorphous (S)-(−)-amisulpride. In some embodiments, the (S)-(−)-amisulpride of step (a) is Form A′ of (S)-(−)-amisulpride.

In some embodiments, the method further comprises drying the enantiomerically pure crystalline form of (S)-(−)amisulpride of step (e).

In some embodiments, the cooling corresponds substantially to the cooling profile shown for the plot of Example 16 in FIG. 10. The recrystallization procedures using the cooling profile of Example 16 can provide for the formation of crystalline solids having narrower and smaller particle size distribution (PSD) compared to other crystallization procedures (see, e.g., Example 16). In some embodiments, the first elevated temperature is from about 45° C. to about 60° C. In some embodiments, the first elevated temperature is from about 50° C. to about 55° C. In some embodiments, the first reduced temperature is from about 38° C. to about 42° C. In some embodiments, the first period of time is about 120 min. In some embodiments, the first period of time is from about 90 min to about 150 min. In some embodiments, the second reduced temperature is from about 10° C. to about 15° C. In some embodiments, the second period of time is about 60 min. In some embodiments, the second period of time is about 30 min to about 90 min. In some embodiments, the cooling over the first period of time is performed at a first cooling rate. In some embodiments, the cooling over the second period of time is performed at a second cooling rate. In some embodiments, the second cooling rate is greater than the first cooling rate. In some embodiments, the first cooling rate is about 0.1° C./min. In some embodiments, the second cooling rate is about 0.5° C./min. In some embodiments, the heating of step (a) is further conducted in the presence of S5, wherein S5 is a solvent. In some embodiments, step (b) further comprises adding S5, wherein S5 is a solvent. In some embodiments, S5 is an ether solvent. In some embodiments, S5 is methyl tert-butyl ether.

In some embodiments, the isolation reagent is of the formula

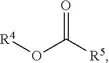

wherein R⁴ is C₁₋₆ alkyl; and R⁵ is C₁₋₅ alkyl or C₁₋₅ alkoxide. In some embodiments, the isolation reagent is of the formula

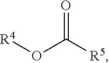

wherein R⁴ is C₃₋₆ alkyl; and R⁵ is C₁₋₅ alkyl. In some embodiments, the isolation reagent is not ethyl acetate. In some embodiments, the isolation reagent is isopropyl acetate, methyl acetate, n-propyl acetate, t-butyl acetate, isobutyl acetate, or n-butyl acetate. In some embodiments, the isolation reagent is isopropyl acetate.

The seed amount of a crystalline form of (S)-(−)-amisulpride can have a greater than about 95% chemical purity and a greater than about 95% enantiomeric purity. The seed amount of a crystalline form of (S)-(−)-amisulpride can have a greater than about 98% chemical purity and a greater than about 98% enantiomeric purity. In some embodiments, the enantiomerically pure crystalline form of (S)-(−)-amisulpride has a greater than about 95% chemical purity. In some embodiments, the enantiomerically pure crystalline form of (S)-(−)-amisulpride has a greater than about 98% chemical purity. In some embodiments, the enantiomerically pure crystalline form of (S)-(−)-amisulpride has a greater than about 99% chemical purity. In some embodiments, the enantiomerically pure crystalline form of (S)-(−)-amisulpride has a greater than about 95% enantiomeric purity. In some embodiments, the enantiomerically pure crystalline form of (S)-(−)-amisulpride has a greater than about 98% enantiomeric purity. In some embodiments, wherein the enantiomerically pure crystalline form of (S)-(−)-amisulpride has a greater than about 99% enantiomeric purity.

The seed amount of a crystalline form of (R)-(+)-amisulpride can have a greater than about 95% chemical purity and a greater than about 95% enantiomeric purity. The seed amount of a crystalline form of (R)-(+)-amisulpride can have a greater than about 98% chemical purity and a greater than about 98% enantiomeric purity. In some embodiments, the enantiomerically pure crystalline form of (R)-(+)-amisulpride has a greater than about 95% chemical purity. In some embodiments, the enantiomerically pure crystalline form of (R)-(+)-amisulpride has a greater than about 98% chemical purity. In some embodiments, the enantiomerically pure crystalline form of (R)-(+)-amisulpride has a greater than about 99% chemical purity. In some embodiments, the enantiomerically pure crystalline form of (R)-(+)-amisulpride has a greater than about 95% enantiomeric purity. In some embodiments, the enantiomerically pure crystalline form of (R)-(+)-amisulpride has a greater than about 98% enantiomeric purity. In some embodiments, the enantiomerically pure crystalline form of (R)-(+)-amisulpride has a greater than about 99% enantiomeric purity.

Provided herein is a method of preparing an enantiomerically pure crystalline form of (R)-(+)-amisulpride consisting essentially of the following steps:
(a) heating (R)-(+)-amisulpride (e.g., crude, amorphous, or Form A of (R)-(+)-amisulpride) in the presence of an isolation reagent to a first elevated temperature to form a crystallization mixture;
(b) adding a seed amount of Form A of (R)-(+)-amisulpride to the crystallization mixture to form a seeded mixture;
(c) cooling the seeded mixture to a first reduced temperature over a first period of time;
(d) cooling the seeded mixture from step (c) to a second reduced temperature over a second period of time; and
(e) filtering the seeded mixture from step (d) to provide an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°.

Provided herein is a method of preparing an enantiomerically pure crystalline form of (R)-(+)-amisulpride consisting of the following steps:
(a) heating (R)-(+)-amisulpride (e.g., crude, amorphous, or Form A of (R)-(+)-amisulpride) in the presence of an isolation reagent to a first elevated temperature to form a crystallization mixture;
(b) adding a seed amount of Form A of (R)-(+)-amisulpride to the crystallization mixture to form a seeded mixture;
(c) cooling the seeded mixture to a first reduced temperature over a first period of time;
(d) cooling the seeded mixture from step (c) to a second reduced temperature over a second period of time; and
(e) filtering the seeded mixture from step (d) to provide an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°.

### Crystalline Forms

Provided herein is an enantiomerically pure crystalline form of (R)-(+)-amisulpride prepared by a method disclosed herein, wherein the crystalline form of (R)-(+)-amisulpride has a greater than about 95% chemical purity and about 95% enantiomeric purity. In some embodiments, the crystalline form of (R)-(+)-amisulpride has a greater than about 98% chemical purity and about 98% enantiomeric purity. In some embodiments, the crystalline form of (R)-(+)-amisulpride has a greater than about 99% chemical purity and about 99% enantiomeric purity.

Also provided herein is an enantiomerically pure crystalline form of (S)-(−)-amisulpride prepared by a method disclosed herein, wherein the crystalline form of (S)-(−)-amisulpride has a greater than about 95% chemical purity and about 95% enantiomeric purity. In some embodiments, the crystalline form of (S)-(−)-amisulpride has a greater than about 98% chemical purity and about 98% enantiomeric purity. In some embodiments, the crystalline form of (S)-(−)-amisulpride has a greater than about 99% chemical purity and about 99% enantiomeric purity.

In some embodiments, provided herein is an enantiomerically pure crystalline form of (R)-(+)-amisulpride or (S)-(−)-amisulpride prepared by a method disclosed herein, comprising less than about 5.0 wt %, about 2.5 wt %, about 1.0 wt %, about 0.5 wt %, about 0.2 wt %, or about 0.1 wt % of a compound of the following formula:

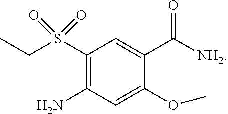 [SMILES: CCS(=O)(=O)c1cc(C(N)=O)c(OC)cc1N]

In some embodiments, provided herein is an enantiomerically pure crystalline form of (R)-(+)-amisulpride or (S)-(−)-amisulpride prepared by a method disclosed herein, comprising less than about 0.2 wt % of a compound of the following formula:

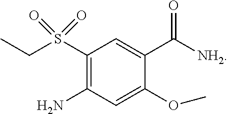 [SMILES: CCS(=O)(=O)c1cc(C(N)=O)c(OC)cc1N]

In some embodiments, provided herein is an enantiomerically pure crystalline form of (R)-(+)-amisulpride or (S)-(−)-amisulpride prepared by a method disclosed herein, comprising less than about 5.0 wt %, about 2.5 wt %, about 1.0 wt %, about 0.5 wt %, about 0.2 wt %, or about 0.1 wt % of a compound of the following formula:

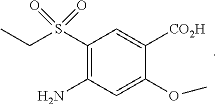 [SMILES: CCS(=O)(=O)c1cc(C(=O)O)c(OC)cc1N]

In some embodiments, provided herein is an enantiomerically pure crystalline form of (R)-(+)-amisulpride or (S)-(−)-amisulpride prepared by a method disclosed herein, comprising less than about 0.2 wt % of a compound of the following formula:

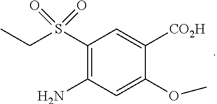 [SMILES: CCS(=O)(=O)c1cc(C(=O)O)c(OC)cc1N]

In some embodiments, provided herein is an enantiomerically pure crystalline form of (R)-(+)-amisulpride or (S)-(−)-amisulpride prepared by a method disclosed herein, comprising less than about 5.0 wt %, about 2.5 wt %, about 1.0 wt %, about 0.5 wt %, about 0.2 wt %, or about 0.1 wt % of a compound of the following formula:

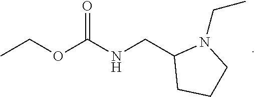

In some embodiments, provided herein is an enantiomerically pure crystalline form of (R)-(+)-amisulpride or (S)-(−)-amisulpride prepared by a method disclosed herein, comprising less than about 0.2 wt % of a compound of the following formula:

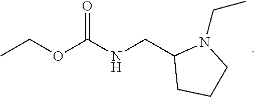

In some embodiments, provided herein is an enantiomerically pure crystalline form of (R)-(+)-amisulpride prepared by a method disclosed herein, comprising less than about 5.0 wt %, about 2.5 wt %, about 1.0 wt %, about 0.5 wt %, about 0.2 wt %, or about 0.1 wt % of a compound of the following formula:

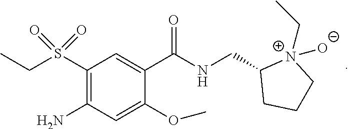 [SMILES: CC[N+]1([O-])CCC[C@@H]1CNC(=O)c1cc(S(=O)(=O)CC)c(N)cc1OC]

In some embodiments, provided herein is an enantiomerically pure crystalline form of (R)-(+)-amisulpride prepared by a method disclosed herein, comprising less than about 0.2 wt % of a compound of the following formula:

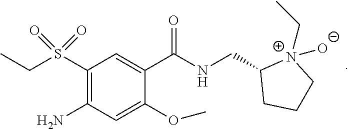 [SMILES: CC[N+]1([O-])CCC[C@@H]1CNC(=O)c1cc(S(=O)(=O)CC)c(N)cc1OC]

In some embodiments, provided herein is an enantiomerically pure crystalline form of (R)-(+)-amisulpride prepared by a method disclosed herein, comprising less than about 0.2 wt % of a compound of the following formula:

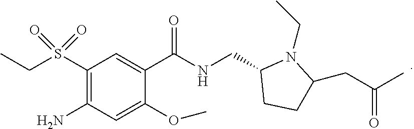 [SMILES: CCN1C(CC(C)=O)CC[C@@H]1CNC(=O)c1cc(S(=O)(=O)CC)c(N)cc1OC]

In some embodiments, provided herein is an enantiomerically pure crystalline form of (R)-(+)-amisulpride prepared by a method disclosed herein, comprising less than about 0.2 wt % of a compound of the following formula:

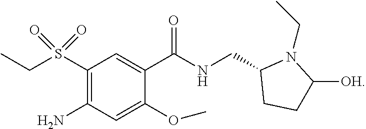 [SMILES: CCN1C(O)CC[C@@H]1CNC(=O)c1cc(S(=O)(=O)CC)c(N)cc1OC]

In some embodiments, provided herein is an enantiomerically pure crystalline form of (S)-(−)-amisulpride prepared by a method disclosed herein, comprising less than about 5.0 wt %, about 2.5 wt %, about 1.0 wt %, about 0.5 wt %, about 0.2 wt %, or about 0.1 wt % of a compound of the following formula:

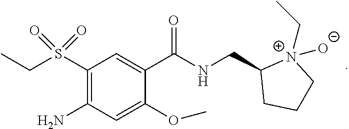 [SMILES: CC[N+]1([O-])CCC[C@H]1CNC(=O)c1cc(S(=O)(=O)CC)c(N)cc1OC]

In some embodiments, provided herein is an enantiomerically pure crystalline form of (S)-(−)-amisulpride prepared by a method disclosed herein, comprising less than about 0.2 wt % of a compound of the following formula:

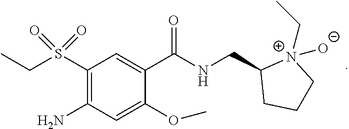 [SMILES: CC[N+]1([O-])CCC[C@H]1CNC(=O)c1cc(S(=O)(=O)CC)c(N)cc1OC]

In some embodiments, provided herein is an enantiomerically pure crystalline form of (S)-(−)-amisulpride prepared by a method disclosed herein, comprising less than about 0.2 wt % of a compound of the following formula:

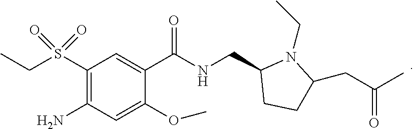 [SMILES: CCN1C(CC(C)=O)CC[C@H]1CNC(=O)c1cc(S(=O)(=O)CC)c(N)cc1OC]

In some embodiments, provided herein is an enantiomerically pure crystalline form of (S)-(−)-amisulpride prepared by a method disclosed herein, comprising less than about 0.2 wt % of a compound of the following formula:

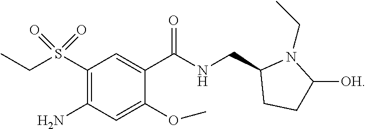 [SMILES: CCN1C(O)CC[C@H]1CNC(=O)c1cc(S(=O)(=O)CC)c(N)cc1OC]

In various embodiments the methods described herein provide enantiomerically pure crystalline forms of (R)-amisulpride of Form A having an enantiomeric purity of greater than about 90%, an enantiomeric purity of greater than about 95%, an enantiomeric purity of greater than about 97%, an enantiomeric purity of greater than about 99%, an enantiomeric purity of greater than about 99.5%, an enantiomeric purity of greater than about 99.7%, or an enantiomeric purity of greater than about 99.9%. In various embodiments these methods provide crystalline forms of (R)-amisulpride of Form A that have a greater than about 90% chemical purity, greater than about 95% chemical purity, greater than about 97% chemical purity, greater than about 99% chemical purity, greater than about 99.5% chemical purity, greater than about 99.7% chemical purity, or greater than about 99.9% chemical purity. In various embodiments, these methods provide crystalline (R)-amisulpride of Form A that has less than about 8000 ppm residual solvents, less than about 6000 ppm residual solvents, less than about 4000 ppm residual solvents, less than about 2000 ppm residual solvents, less than about 1000 ppm residual solvents, less than about 800 ppm residual solvents, or less than about 500 ppm residual solvents.

In various embodiments the methods described herein provide enantiomerically pure crystalline forms of (S)-amisulpride of Form A′ having an enantiomeric purity of greater than about 90%, an enantiomeric purity of greater than about 95%, an enantiomeric purity of greater than about 97%, an enantiomeric purity of greater than about 99%, an enantiomeric purity of greater than about 99.5%, an enantiomeric purity of greater than about 99.7%, or an enantiomeric purity of greater than about 99.9%. In various embodiments these methods provide crystalline forms of (S)-amisulpride of Form A′ that have a greater than about 90% chemical purity, greater than about 95% chemical purity, greater than about 97% chemical purity, greater than about 99% chemical purity, greater than about 99.5% chemical purity, greater than about 99.7% chemical purity, or greater than about 99.9% chemical purity. In various embodiments, these methods provide crystalline (S)-amisulpride of Form A′ that has less than about 8000 ppm residual solvents, less than about 6000 ppm residual solvents, less than about 4000 ppm residual solvents, less than about 2000 ppm residual solvents, less than about 1000 ppm residual solvents, less than about 800 ppm residual solvents, or less than about 500 ppm residual solvents.

In some embodiments, the methods herein provide an enantiomerically pure crystalline form of (R)-(+)-amisulpride, wherein the crystalline form of (R)-(+)-amisulpride has a particle size distribution D₁₀ value of less than about 15 μm, as measured by laser diffraction particle size analysis. In some embodiments, the particle size distribution D₁₀ value is less than about 10 μm, as measured by laser diffraction particle size analysis. In some embodiments, the particle size distribution D₅₀ value is less than about 50 μm, as measured by laser diffraction particle size analysis. In some embodiments, the particle size distribution D₅₀ value is less than about 30 μm, as measured by laser diffraction particle size analysis. In some embodiments, the particle size distribution D₉₀ value is less than about 150 μm, as measured by laser diffraction particle size analysis. In some embodiments, the particle size distribution D₉₀ value is less than about 100 μm, as measured by laser diffraction particle size analysis. In some embodiments, the crystalline form of (R)-(+)-amisulpride has a particle size distribution D₁₀ value of less than about 10 μm; a particle size distribution D₅₀ value of less than about 50 μm; and a particle size distribution D₉₀ value of less than about 150 μm, as measured by laser diffraction particle size analysis. In some embodiments, the crystalline form of (R)-(+)-amisulpride has a particle size distribution D₁₀ value of less than about 10 μm; a particle size distribution D₅₀ value of less than about 30 μm; and a particle size distribution D₉₀ value of less than about 100 μm, as measured by laser diffraction particle size analysis.

In some embodiments, the methods herein provide an enantiomerically pure crystalline form of (R)-(+)-amisulpride, wherein the crystalline form of (R)-(+)-amisulpride has a particle size distribution D₁₀ value of about 2 to about 20 μm, as measured by laser diffraction particle size analysis. In some embodiments, the particle size distribution D₁₀ value is about 2 to about 10 μm, as measured by laser diffraction particle size analysis. In some embodiments, the particle size distribution D₅₀ value is about 5 to about 50 μm, as measured by laser diffraction particle size analysis. In some embodiments, the particle size distribution D₅₀ value is about 15 to about 30 μm, as measured by laser diffraction particle size analysis. In some embodiments, the particle size distribution D₉₀ value of about 40 to about 150 μm, as measured by laser diffraction particle size analysis. In some embodiments, the particle size distribution D₉₀ value of about 75 to about 100 μm, as measured by laser diffraction particle size analysis. In some embodiments, the crystalline form of (R)-(+)-amisulpride has a particle size distribution D₁₀ value of about 2 to about 10 μm; a particle size distribution D₅₀ value of about 5 to about 50 μm; and a particle size distribution D₉₀ value of about 40 to about 150 μm, as measured by laser diffraction particle size analysis. In some embodiments, the crystalline form of (R)-(+)-amisulpride has a particle size distribution D₁₀ value of about 2 to about 10 μm; a particle size distribution D₅₀ value of about 15 to about 30 μm; and a particle size distribution D₉₀ value of about 75 to about 100 μm, as measured by laser diffraction particle size analysis.

In some embodiments, the methods herein provide an enantiomerically pure crystalline form of (S)-(−)-amisulpride, wherein the crystalline form of (S)-(−)-amisulpride has a particle size distribution D₁₀ value of less than about 15 μm, as measured by laser diffraction particle size analysis. In some embodiments, the particle size distribution D₁₀ value is less than about 10 μm, as measured by laser diffraction particle size analysis. In some embodiments, the particle size distribution D₅₀ value is less than about 50 μm, as measured by laser diffraction particle size analysis. In some embodiments, the particle size distribution D₅₀ value is less than about 30 μm, as measured by laser diffraction particle size analysis. In some embodiments, the particle size distribution D₉₀ value is less than about 150 μm, as measured by laser diffraction particle size analysis. In some embodiments, the particle size distribution D₉₀ value is less than about 100 μm, as measured by laser diffraction particle size analysis. In some embodiments, the crystalline form of (S)-(−)-amisulpride has a particle size distribution D₁₀ value of less than about 10 μm; a particle size distribution D₅₀ value of less than about 50 μm; and a particle size distribution D₉₀ value of less than about 150 μm, as measured by laser diffraction particle size analysis. In some embodiments, the crystalline form of (S)-(−)-amisulpride has a particle size distribution D₁₀ value of less than about 10 μm; a particle size distribution D₅₀ value of less than about 30 μm; and a particle size distribution D₉₀ value of less than about 100 μm, as measured by laser diffraction particle size analysis.

In some embodiments, the methods herein provide an enantiomerically pure crystalline form of (S)-(−)-amisulpride, wherein the crystalline form of (S)-(−)-amisulpride has a particle size distribution D₁₀ value of about 2 to about 20 μm, as measured by laser diffraction particle size analysis. In some embodiments, the particle size distribution D₁₀ value is about 2 to about 10 μm, as measured by laser diffraction particle size analysis. In some embodiments, the particle size distribution D₅₀ value is about 5 to about 50 μm, as measured by laser diffraction particle size analysis. In some embodiments, the particle size distribution D₅₀ value is about 15 to about 30 μm, as measured by laser diffraction particle size analysis. In some embodiments, the particle size distribution D₉₀ value of about 40 to about 150 μm, as measured by laser diffraction particle size analysis. In some embodiments, the particle size distribution D₉₀ value of about 75 to about 100 μm, as measured by laser diffraction particle size analysis. In some embodiments, the crystalline form of (S)-(−)-amisulpride has a particle size distribution D₁₀ value of about 2 to about 10 μm; a particle size distribution D₅₀ value of about 5 to about 50 μm; and a particle size distribution D₉₀ value of about 40 to about 150 μm, as measured by laser diffraction particle size analysis. In some embodiments, the crystalline form of (S)-(−)-amisulpride has a particle size distribution D₁₀ value of about 2 to about 10 μm; a particle size distribution D₅₀ value of about 15 to about 30 μm; and a particle size distribution D₉₀ value of about 75 to about 100 μm, as measured by laser diffraction particle size analysis.

In various embodiments, the methods herein provide a crystalline form of (R)-amisulpride characterized by the following properties, an XRPD pattern comprising peaks, in terms of 2-theta, at 7.0±0.2°, 9.7±0.2°, and 15.4±0.2° and one or more of the following:
(a) the powder x-ray diffraction pattern further comprising peaks, in terms of 2-theta, at 9.3±0.2° and 19.4±0.2°;
(b) the powder x-ray diffraction 14.9±0.2°, 16.9±0.2°, and 20.1±0.2°; and pattern further comprising peaks, in terms of 2-theta, and
(c) the powder x-ray diffraction pattern further comprising peaks, in terms of 2-theta, at 21.0±0.2°, and 23.2±0.2°.

In various embodiments, the methods herein provide a crystalline form of (S)-amisulpride characterized by the following properties, an XRPD pattern comprising peaks, in terms of 2-theta, at 7.0±0.2°, 9.7±0.2°, and 15.4±0.2° and one or more of the following:
(a) the powder x-ray diffraction pattern further comprising peaks, in terms of 2-theta, at 9.3±0.2° and 19.4±0.2°;
(b) the powder x-ray diffraction 14.9±0.2°, 16.9±0.2°, and 20.1±0.2°; and pattern further comprising peaks, in terms of 2-theta, and
(c) the powder x-ray diffraction pattern further comprising peaks, in terms of 2-theta, at 21.0±0.2°, and 23.2±0.2°.

In various embodiments, the methods herein provide (R)-amisulpride of crystalline Form A characterized by an XRPD pattern substantially in accord with FIG. 1A and/or FIG. 6A. An XRPD pattern substantially in accord with FIG. 1A or FIG. 6A includes, but is not limited to, an XRPD pattern comprising peaks, in terms of 2-theta, at 7.0±0.2° and 9.7±0.2°, and further peaks at 15.4±0.2° and/or 19.4±0.2°. In various embodiments, the methods herein provide a crystalline form of (R)-amisulpride characterized by three or more peaks in its XRPD pattern selected from those at 7.0±0.2°, 9.7±0.2°, 15.4±0.2°, 16.9±0.2°, 19.4±0.2°, 20.1±0.2°, 21.0±0.2°, 23.2±0.2°, and 29.3±0.2°, in terms of 2-theta.

In various embodiments, the methods herein provide (S)-amisulpride of crystalline Form A′ characterized by an XRPD pattern substantially in accord with FIG. 2A and/or FIG. 6B. An XRPD pattern substantially in accord with FIG. 2A or FIG. 6B includes, but is not limited to, an XRPD pattern comprising peaks, in terms of 2-theta, at 7.0±0.2° and 9.7±0.2°, and further peaks at 15.4±0.2° and/or 19.4±0.2°. In various embodiments, the methods herein provide a crystalline form of (S)-amisulpride characterized by three or more peaks in its XRPD pattern selected from those at 7.0±0.2°, 9.7±0.2°, 15.4±0.2°, 16.9±0.2°, 19.4±0.2°, 20.1±0.2°, 21.0±0.2°, 23.2±0.2°, and 29.3±0.2°, in terms of 2-theta.

In various embodiments, the methods describe herein provide a crystalline form of enantiomeric amisulpride that is the substantially non-hygroscopic. In various embodiments, the methods provide a crystalline (R)-amisulpride of Form A that, as measured by dynamic vapor sorption (DVS), at 25° C. scanned over 0 to 95% relative humidity, has a maximum mass change in water sorption isotherms of less than about (i) 1%, (ii) 0.5%, (iii) 0.3%, or (iv) 0.2%. In various embodiments, the methods describe herein provide a crystalline form of enantiomeric amisulpride that is the substantially non-hygroscopic. In various embodiments, the methods provide a crystalline (S)-amisulpride of Form A′ that, as measured by dynamic vapor sorption (DVS), at 25° C. scanned over 0 to 95% relative humidity, has a maximum mass change in water sorption isotherms of less than about (i) 1%, (ii) 0.5%, (iii) 0.3%, or (iv) 0.2%.

In various embodiments, the methods describe herein provide a crystalline form of (R)-amisulpride characterized by the following properties, an XRPD pattern comprising peaks, in terms of 2-theta, at 7.0±0.2° and 9.7±0.2°, and further peaks at 15.4±0.2° and/or 19.4±0.2°, and an enantiomeric purity of greater than about 99%, a chemical purity greater than about 99%, a residual solvent content of less than about 800 ppm, and is substantially non-hygroscopic.

In various embodiments, the methods describe herein provide a crystalline form of (S)-amisulpride characterized by the following properties, an XRPD pattern comprising peaks, in terms of 2-theta, at 7.0±0.2° and 9.7±0.2°, and further peaks at 15.4±0.2° and/or 19.4±0.2°, and an enantiomeric purity of greater than about 99%, a chemical purity greater than about 99%, a residual solvent content of less than about 800 ppm, and is substantially non-hygroscopic.

### Pharmaceutical Compositions

According to an embodiment, the disclosure provides a composition comprising crystalline form disclosed herein and a pharmaceutically acceptable carrier. In some embodiments, the amount of the crystalline form in compositions of this disclosure is such that is effective to treat, prevent, and/or manage various neurological and/or psychiatric diseases, disorders and/or symptoms in a subject. In some embodiments, a composition of this disclosure is formulated for administration to a subject in need of such composition. In some embodiments, a composition of this disclosure is formulated for oral administration to a subject.

As used herein, the term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

In some embodiments the pharmaceutical compositions of the present inventions comprise one or more pharmaceutically acceptable excipients, including, but not limited to, one or more binders, bulking agents, buffers, stabilizing agents, surfactants, wetting agents, lubricating agents, diluents, viscosity enhancing or reducing agents, emulsifiers, suspending agents, preservatives, antioxidants, opaquing agents, glidants, processing aids, colorants, sweeteners, taste-masking agents, perfuming agents, flavoring agents, diluents and other known additives to provide an elegant presentation of the drug or aid in the manufacturing of a medicament or pharmaceutical product comprising a composition of the present inventions. Examples of carriers and excipients well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005.

In various embodiments, the compositions are formulated with one or more pharmaceutically acceptable excipients in accordance with known and established practice. Thus, in various embodiments the compositions are formulated as, for example, a liquid, powder, granules, elixir, injectable solution, or suspension. Formulations for oral use are preferred and may be provided, for instance, as tablets, caplets, or capsules, wherein the pharmacologically active ingredients are mixed with an inert solid diluent. Tablets may also include granulating and disintegrating agents, and may be coated or uncoated. Formulations for topical use may be provided, for example as topical solutions, lotions, creams, ointments, gels, foams, patches, powders, solids, sponges, tapes, vapors, pastes or tinctures.

The amount of the crystalline forms of the disclosure that may be combined with the carrier materials to produce a composition in a single dosage form will vary depending upon a variety of factors, including the host treated and the particular mode of administration. It should also be understood that a specific dosage and treatment regimen for any particular subject will depend upon a variety of factors, including the age of the subject, body weight of subject, general health of the subject, sex of the subject, time of administration, rate of excretion, drug combination, and the judgment of the treating physician and the severity of the particular disease being treated.

Provided herein is a pharmaceutical composition comprising the enantiomerically pure crystalline form of (R)-(+)-amisulpride prepared by a method disclosed herein, and a pharmaceutically acceptable carrier. In some embodiments, the pharmaceutical composition further comprises the enantiomerically pure crystalline form of (S)-(−)-amisulpride prepared by a method disclosed herein. In some embodiments, the crystalline form of (R)-(+)-amisulpride prepared by a method disclosed herein and the crystalline form of (S)-(−)-amisulpride prepared by a method disclosed herein are in a ratio of about 65:35 to about 88:12 by weight of free base. In some embodiments, the crystalline form of (R)-(+)-amisulpride and the crystalline form of (S)-(−)-amisulpride are in a ratio of about 75:25 to about 88:12 by weight of free base. In some embodiments, the crystalline form of (R)-(+)-amisulpride and the crystalline form of (S)-(−)-amisulpride are in a ratio of about 80:20 to about 88:12 by weight of free base. In some embodiments, the crystalline form of (R)-(+)-amisulpride and the crystalline form of (S)-(−)-amisulpride are in a ratio of about 85:15 by weight of free base.

In some embodiments, the pharmaceutical composition comprises less than about 1.0 wt % of a compound of the following formula:

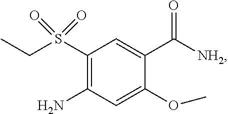 [SMILES: CCS(=O)(=O)c1cc(C(N)=O)c(OC)cc1N]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 0.2 wt % of a compound of the following formula:

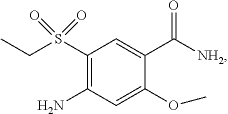 [SMILES: CCS(=O)(=O)c1cc(C(N)=O)c(OC)cc1N]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 1.0 wt % of a compound of the following formula:

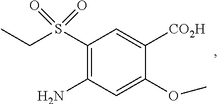 [SMILES: CCS(=O)(=O)c1cc(C(=O)O)c(OC)cc1N]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 0.2 wt % of a compound of the following formula:

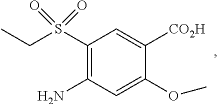 [SMILES: CCS(=O)(=O)c1cc(C(=O)O)c(OC)cc1N]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 1.0 wt % of a compound of the following formula:

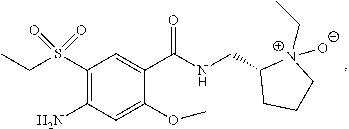 [SMILES: CC[N+]1([O-])CCC[C@@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 0.2 wt % of a compound of the following formula:

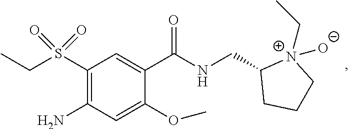 [SMILES: CC[N+]1([O-])CCC[C@@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 1.0 wt % of a compound of the following formula:

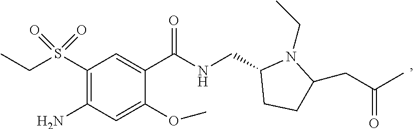 [SMILES: CCN1C(CC(C)=O)CC[C@@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 0.2 wt % of a compound of the following formula:

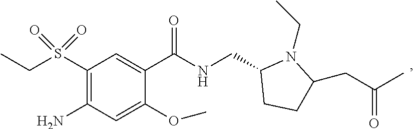 [SMILES: CCN1C(CC(C)=O)CC[C@@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 1.0 wt % of a compound of the following formula:

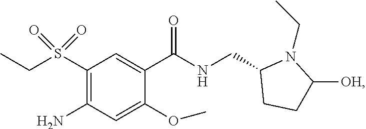 [SMILES: CCN1C(O)CC[C@@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 0.2 wt % of a compound of the following formula:

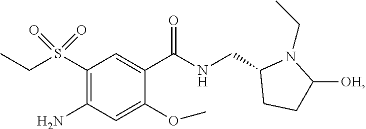 [SMILES: CCN1C(O)CC[C@@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 1.0 wt % of a compound of the following formula:

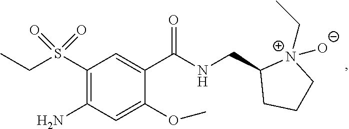 [SMILES: CC[N+]1([O-])CCC[C@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 0.2 wt % of a compound of the following formula:

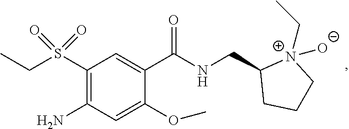 [SMILES: CC[N+]1([O-])CCC[C@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 1.0 wt % of a compound of the following formula:

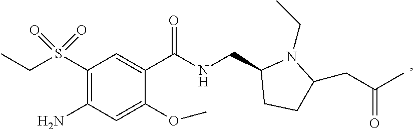 [SMILES: CCN1C(CC(C)=O)CC[C@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 0.2 wt % of a compound of the following formula:

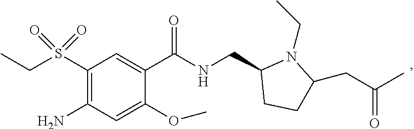 [SMILES: CCN1C(CC(C)=O)CC[C@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 1.0 wt % of a compound of the following formula:

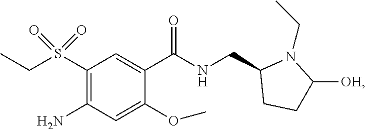 [SMILES: CCN1C(O)CC[C@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 0.2 wt % of a compound of the following formula:

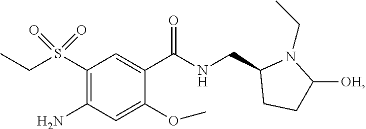 [SMILES: CCN1C(O)CC[C@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

In some embodiments, the pharmaceutical composition comprises less than about 1.0 wt % of a compound of the following formula:

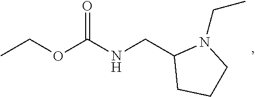

wherein the recited wt % is calculated relative to the total amount of (R)-(+)-amisulpride and (S)-(−)-amisulpride that is present in the pharmaceutical composition.

In some embodiments, the pharmaceutical composition comprises less than about 0.2 wt % of a compound of the following formula:

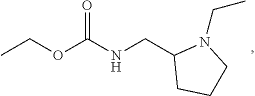

wherein the recited wt % is calculated relative to the total amount of (R)-(+)-amisulpride and (S)-(−)-amisulpride that is present in the pharmaceutical composition.

Provided herein is a pharmaceutical composition comprising the enantiomerically pure crystalline form of (S)-(−)-amisulpride prepared by a method disclosed herein, and a pharmaceutically acceptable carrier. Also provided herein is a pharmaceutical composition comprising the enantiomerically pure crystalline form of (R)-(+)-amisulpride prepared by a method disclosed herein, and a pharmaceutically acceptable carrier.

### Methods of Use

The crystalline forms provided herein can be used to treat, and/or used to manufacture a medicament to treat, a psychiatric disorder in a subject, a neurological disorder in a subject, or both a neurological disorder and a psychiatric disorder, the disorder including, but not limited to, one or more of a mood disorder, bipolar disorder (BPD), depression, bipolar depression, major depressive episodes associated with bipolar I disorder, major depressive disorder (MDD), as an adjunctive treatment of major depressive disorder; major depressive disorder with mixed features (MDD-MF), treatment resistant depression (TRD), schizophrenia, negative symptoms of schizophrenia, and schizoaffective disorder.

In some embodiments, provided herein is a method of treating a psychiatric disorder in a subject comprising administering to the subject an enantiomerically pure crystalline form of (R)-(+)-amisulpride or (S)-(−)-amisulpride prepared by a method disclosed herein, or a pharmaceutical composition disclosed herein. In some embodiments, provided herein is a method of treating a psychiatric disorder in a subject comprising administering to the subject an enantiomerically pure crystalline form of (R)-(+)-amisulpride and/or (S)-(−)-amisulpride prepared by a method disclosed herein, or a pharmaceutical composition disclosed herein. In some embodiments, the psychiatric disorder is one or more of a mood disorder, bipolar disorder (BPD), depression, bipolar depression, major depressive disorder (MDD), as an adjunctive treatment of major depressive disorder, major depressive disorder with mixed features (MDD-MF), treatment resistant depression (TRD), schizophrenia, negative symptoms of schizophrenia, and schizoaffective disorder. In some embodiments, the psychiatric disorder is a depressive order. In some embodiments, the psychiatric disorder is bipolar disorder. In some embodiments, the psychiatric disorder is bipolar depression. In some embodiments, the psychiatric disorder is major depressive disorder (MDD). In some embodiments, the psychiatric disorder is major depressive disorder with mixed features (MDD-MF). In some embodiments, the psychiatric disorder is treatment resistant depression (TRD). In some embodiments, the psychiatric disorder is schizophrenia.

### EXAMPLES

Non-limiting examples of various embodiments of making crystalline enantiomerically pure amisulpride of Form A and Form A′, or characterized by an XRPD pattern comprising peaks, in terms of 2-theta, at least at 7.0±0.2° and 9.7±0.2°, and further peaks at 15.4±0.2° and/or 19.4±0.2°, are further illustrated and described in Examples 1-15.

Aspects, embodiments, and features of the inventions may be further understood from the following examples, which should not be construed as limiting the scope of the inventions.

Example 1 provides a general reaction scheme (Scheme 1A) to the preparation of (S)-amisulpride of Form A′. Example 2 provides an example of the application of Scheme 1A of Example 1 to prepare a lab scale batch (e.g., under 100 g) of (S)-amisulpride of Form A′. Example 4 provides an example of the application of Scheme 1A of Example 1 to prepare a more manufacturing scale batch (˜25 kg) of (S)-amisulpride of Form A′. Example 10 provides an example of the application of Scheme 1A of Example 1, using alternative conditions, to prepare (S)-amisulpride of Form A′. Example 11 provides an example of the application of Scheme 1A of Example 1, using alternative conditions, to prepare a lab scale batch (S)-amisulpride of Form A′. Example 14 provides an example of the application of Scheme 1A of Example 1, using alternative conditions, to prepare a manufacturing scale batch (200 kg) of (S)-amisulpride of Form A′.

Example 3 provides a general reaction scheme (Scheme 1B) to the preparation of (R)-amisulpride of Form A. Example 5 provides an example of the application of Scheme 1B of Example 3 to prepare a more manufacturing scale batch (˜25 kg) of (R)-amisulpride of Form A. Example 12 provides an example of the application of Scheme 1B of Example 3, using alternative conditions, to prepare (R)-amisulpride of Form A. Example 13 provides an example of the application of Scheme 1B of Example 3, using alternative conditions, to prepare a lab scale batch (R)-amisulpride of Form A. Example 15 provides an example of the application of Scheme 1B of Example 1, using alternative conditions, to prepare a manufacturing scale batch (200 kg) of (R)-amisulpride of Form A.

Example 6 provides a general reaction scheme (Scheme 2A) to the preparation of (S)-amisulpride of Form A′.

Example 7 provides a general reaction scheme (Scheme 2B) to the preparation of (R)-amisulpride of Form A. Example 8 provides an example of the application of Scheme 2B of Example 7 to prepare a lab scale batch (e.g., under 100 g) of (R)-amisulpride of Form A.

Example 9 provides characterization data of crystalline amisulpride enantiomers made substantially according to Example 4 and Example 5.

Example 16 provides PSD characterization of crystalline (R) and (S)-amisulpride and formulations comprising (R) and (S)-amisulpride.

### Example 1: General Method of Making (S)-4-Amino-N-[(1-ethyl-2-pyrrolidinyl)methyl]-5-(ethylsulfonyl)-2-methoxybenzamide of Form A′

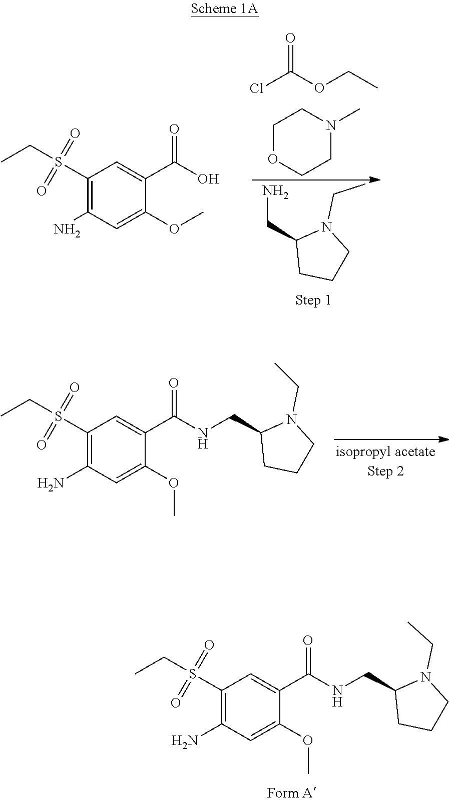

Scheme 1A, Step 1: (S)-4-Amino-N-[(1-ethyl-2-pyrrolidinyl)methyl]-5-(ethylsulfonyl)-2-methoxybenzamide (crude freebase): To a mixture of 4-amino-5-(ethylsulfonyl)-2-methoxybenzoic acid in acetone at −10±5° C. is added ethyl chloroformate. Then 4-methylmorpholine is added at a rate (reaction is exothermic) so as to maintain the internal temperature below 0° C. The reaction is stirred for 1 hour at −10° C. and then (S)-(1-ethyl pyrrolidin-2-yl)methanamine is added. After stirring for about 2 hours (and a minimum of 1 hour) the reaction mixture is concentrated and diluted with aqueous potassium carbonate and isopropyl acetate. The aqueous layer is removed and the organic layer is washed with water twice. The water is removed via azeotropic distillation until the water content of the isopropyl acetate solution is below 0.5 wt %. The solution is concentrated to approximately 35 wt % and the temperature adjusted to 45° C. The solution is seeded at 31° C. with about 2 wt % (S)-4-Amino-N-[(1-ethyl-2-pyrrolidinyl)methyl]-5-(ethylsulfonyl)-2-methoxybenzamide of Form A′ and stirred at 45° C. for 30 min. The mixture is cooled to 15° C. and stirred for 1 h. The suspension is then filtered and the product cake is washed with isopropyl acetate. The wet-cake is dried under vacuum at 40° C.±5° C. to constant weight to yield (S)-4-amino-N-[(1-ethyl-2-pyrrolidinyl)methyl]-5-(ethylsulfonyl)-2-methoxybenzamide (crude freebase).

Scheme 1A, Step 2 (recrystallization): (S)-4-Amino-N-[(1-ethyl-2-pyrrolidinyl)methyl]-5-(ethylsulfonyl)-2-methoxybenzamide substantially (Form A′): Isopropyl acetate is added to (S)-4-Amino-N-[(1-ethyl-2-pyrrolidinyl)methyl]-5-(ethylsulfonyl)-2-methoxybenzamide (crude freebase) and the mixture is heated to 55° C. to achieve dissolution. The resulting solution is then passed through a filter. The filtrate is concentrated by distillation and the resulting solution is cooled to 50° C. and seeded with Form A′. The resulting slurry is stirred at 50° C. for 1.5 hours and then slowly cooled to 15° C. The suspension is then filtered and the product cake is washed with isopropyl acetate. The wet-cake is dried under vacuum at 40° C.±5° C. to constant weight to yield (S)-4-amino-N-[(1-ethyl-2-pyrrolidinyl)methyl]-5-(ethylsulfonyl)-2-methoxybenzamide substantially of Form A′.

Example 2: (S)-4-Amino-N-[(1-ethyl-2-pyrrolidinyl)methyl]-5-(ethylsulfonyl)-2-methoxybenzamide of Form A′; Scheme 1A Lab Scale:

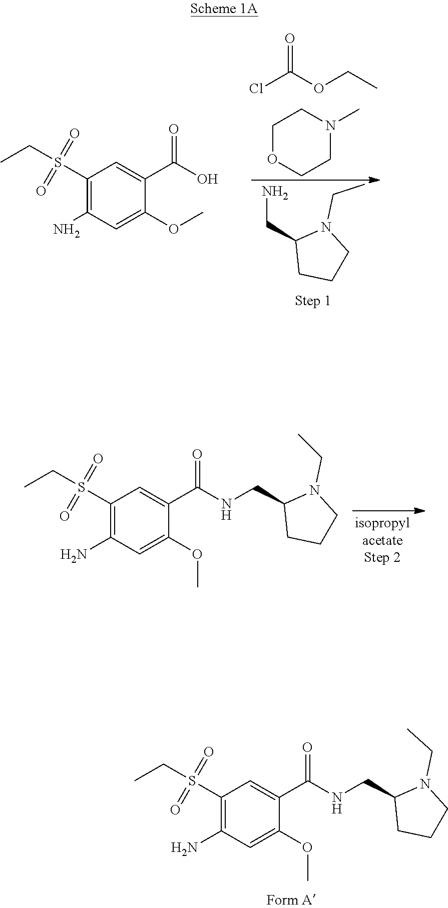

Example 2, Scheme 1A, Step 1: (S)-4-Amino-N-[(1-ethyl-2-pyrrolidinyl)methyl]-5-(ethylsulfonyl)-2-methoxybenzamide (crude freebase): To a mixture of 45 g of 4-amino-5-(ethylsulfonyl)-2-methoxybenzoic acid in 240 g of acetone at −10±5° C. was added 21.2 g of ethyl chloroformate. 22.5 g of 4-methylmorpholine was then added over 1 h. The reaction was stirred for 1 hour at −10° C. and then 23.9 g of (S)-(1-ethyl pyrrolidin-2-yl)methanamine was added. The reaction was stirred for 2 hours and concentrated to approximately 90 mL. The mixture was diluted with 213 g of 26 wt % aqueous potassium carbonate and 225 g of isopropyl acetate. The mixture was stirred for 30 min and the aqueous layer was removed. The organic layer was washed with 90 g of water twice. The isopropyl acetate solution (i.e., the organic layer) was dried via azeotropic distillation until the water content of the isopropyl acetate solution was below 0.5% by weight. The solution was concentrated to approximately 35 wt % and the temperature was adjusted to 45° C. The solution was seeded at 45° C. with 1 wt % (S)-4-Amino-N-[(1-ethyl-2-pyrrolidinyl)methyl]-5-(ethylsulfonyl)-2-methoxybenzamide of Form A′ and stirred at 45° C. for 30 min. The mixture was slowly cooled to 15° C. and stirred for 1 h. The suspension was then filtered and the product cake was washed with 54 g isopropyl acetate. The wet-cake was dried under vacuum at 40° C.±5° C. to constant weight to yield approximately 46 g of (S)-4-Amino-N-[(1-ethyl-2-pyrrolidinyl)methyl]-5-(ethylsulfonyl)-2-methoxybenzamide (crude freebase).

Example 2, Scheme 1A, Step 2 (recrystallization): (S)-4-Amino-N-[(1-ethyl-2-pyrrolidinyl)methyl]-5-(ethylsulfonyl)-2-methoxybenzamide substantially (Form A′): Isopropyl acetate (205 g) was added to 51.4 g of (S)-4-Amino-N-[(1-ethyl-2-pyrrolidinyl)methyl]-5-(ethylsulfonyl)-2-methoxybenzamide and the mixture was heated to 50° C. to achieve dissolution. The resulting solution was then passed through a filter. The filtrate was concentrated by distillation to a 35 wt % solution and the temperature of the resulting solution adjusted to 50° C., and seeded with 0.77 g of (S)-4-Amino-N-[(1-ethyl-2-pyrrolidinyl)methyl]-5-(ethylsulfonyl)-2-methoxybenzamide substantially of Form A′, and the mixture was stirred for 1 h. The resulting slurry was cooled to 15° C. over 1 h and stirred for 1.5 h at this temperature. The suspension was then filtered and the product cake was washed with 51 g of isopropyl acetate. The wet-cake was dried under vacuum at 40° C.±5° C. to constant weight to yield 45 g of (S)-4-Amino-N-[(1-ethyl-2-pyrrolidinyl)methyl]-5-(ethylsulfonyl)-2-methoxybenzamide substantially as Form A′.

### Example 3: General Method of Making (R)-4-Amino-N-[(1-ethyl-2-pyrrolidinyl)methyl]-5-(ethylsulfonyl)-2-methoxybenzamide of Form A

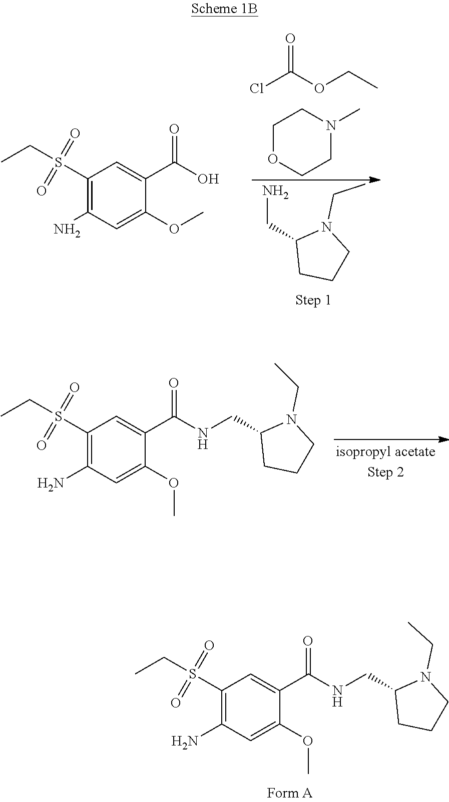

Scheme 1B, Step 1: (R)-4-Amino-N-[(1-ethyl-2-pyrrolidinyl)methyl]-5-(ethylsulfonyl)-2-methoxybenzamide (crude freebase): To a mixture of 4-amino-5-(ethylsulfonyl)-2-methoxybenzoic acid in acetone at −10±5° C. is added ethyl chloroformate, 4-Methylmorpholine is added at a rate (reaction is exothermic) so as to maintain the internal temperature below 0° C. The reaction is stirred for 1 hour at −10° C. and then (R)-(1-ethyl pyrrolidin-2-yl)methanamine is added. After stirring for 2 hours the reaction mixture is concentrated and diluted with aqueous potassium carbonate and isopropyl acetate. The aqueous layer is removed and the organic layer is washed with water twice. The water is removed via azeotropic distillation until the water content of the isopropyl acetate solution is below 0.5%. The solution is concentrated to approximately 35 wt % and the temperature adjusted to 30-35° C. The solution is seeded at 31° C. with 1 wt % (R)-4-Amino-N-[(1-ethyl-2-pyrrolidinyl)methyl]-5-(ethylsulfonyl)-2-methoxybenzamide of Form A and stirred at 31° C. for 2 h. The mixture is cooled to 20° C. and stirred for 1 h. The suspension is then filtered and the product cake is washed with isopropyl acetate. The wet-cake is dried under vacuum at 40° C.±5° C. to constant weight to yield (R)-4-amino-N-[(1-ethyl-2-pyrrolidinyl)methyl]-5-(ethylsulfonyl)-2-methoxybenzamide (crude freebase).

Scheme 1B, Step 2 (recrystallization): (R)-4-Amino-N-[(1-ethyl-2-pyrrolidinyl)methyl]-5-(ethylsulfonyl)-2-methoxybenzamide substantially (Form A): Isopropyl acetate is added to (R)-4-Amino-N-[(1-ethyl-2-pyrrolidinyl)methyl]-5-(ethylsulfonyl)-2-methoxybenzamide (crude freebase) and the mixture is heated to 50° C. to achieve dissolution. The resulting solution is then passed through a filter. The filtrate is concentrated by distillation and the resulting solution is cooled to 28° C. and seeded with Form A. The resulting slurry is cooled to 23° C. and stirred for 1.5 h at this temperature. The suspension is then filtered and the product cake is washed with isopropyl acetate. The wet-cake is dried under vacuum at 40° C.±5° C. to constant weight to yield (R)-4-amino-N-[(1-ethyl-2-pyrrolidinyl)methyl]-5-(ethylsulfonyl)-2-methoxybenzamide substantially of Form A.

### Example 4: (S)-4-Amino-N-[(1-ethyl-2-pyrrolidinyl)methyl]-5-(ethylsulfonyl)-2-methoxybenzamide of Form A′; Scheme 1A (Scaled-Up)

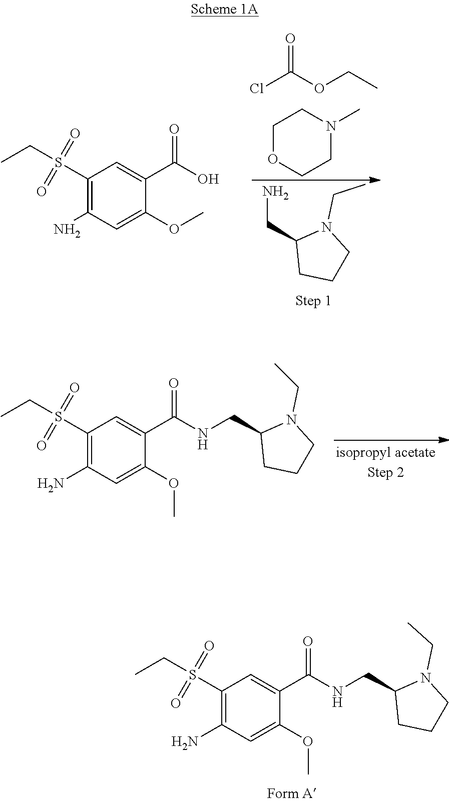

Example 4, Step 1: (S)-4-Amino-N-[(1-ethyl-2-pyrrolidinyl)methyl]-5-(ethylsulfonyl)-2-methoxybenzamide (crude freebase): First 30.0 kg of 4-amino-5-ethylsulfonyl)-2-methoxybenzoic acid and 159 kg of acetone were charged to a 400 L reactor. The mixture was cooled to −10° C. and 14 kg of ethyl chloroformate and 7 kg of acetone rinse was added to the mixture. Then 15 kg of 4-methylmorpholine and a 4 kg acetone rinse was slowly charged to the reactor (reaction is exothermic) while maintaining the temperature below 0° C. Then 15.9 kg of (S)-(1-ethylpyrrolidin-2-yl)methanamine was slowly added to the reactor while maintaining the temperature below 5° C. The reaction was stirred at 5° C. for 1 h and then heated to 22° C. The mixture was stirred for another 12 h at 20° C. and concentrated under reduced pressure to 60 L. The temperature was then adjusted to 22° C. and a solution of 26 wt % potassium carbonate (142 kg) and 150 kg isopropyl acetate was added to the mixture. The reaction mixture was stirred for about 10 min, the stirring stopped, and the layers allowed to separate. The aqueous layer was removed, and 60.0 kg of water added to the organic layer. The reaction mixture was stirred for 10 min, the stirring stopped, and the layers allowed to separate. The aqueous layer was removed, and 60.0 kg of water added to the organic layer. The reaction mixture was stirred for 10 min, the stirring stopped, and the layers allowed to separate. The aqueous layer was removed and the reactor was equipped with a Dean Stark apparatus. The solution was then stirred at reflux with the Dean Stark apparatus removing water. The reactor temperature reached 91.8° C. as the water was removed. After the removal was almost completed, the solution was concentrated to a 45 wt % solution. The reaction temperature was adjusted to 45° C. and the water content was measured and found to be <0.2% by Karl Fischer titration. Then 25 kg of isopropyl acetate was added and the solution was stirred at 45° C. and seeded with 0.3 kg of (S)-amisulpride of Form A′. The mixture was stirred for 30 min at 45° C. and the resulting slurry was cooled to 15° C. over 3.5 h. The slurry was then stirred at 15° C. for 2 h and filtered. The mother liquors from the filtration were recirculated through the reactor and filter. The isolated solid was washed with 36 kg of isopropyl acetate at 15° C. The solid was dried in a filter dryer at 35° C. to yield 35.2 kg of (S)-4-Amino-N-[(1-ethyl-2-pyrrolidinyl)methyl]-5-(ethylsulfonyl)-2-methoxybenzamide (crude freebase).

Example 4, Step 2 (recrystallization): (S)-4-Amino-N-[(1-ethyl-2-pyrrolidinyl)methyl]-5-(ethylsulfonyl)-2-methoxybenzamide substantially (Form A′): The 35.2 kg of (S)-4-Amino-N-[(1-ethyl-2-pyrrolidinyl)methyl]-5-(ethylsulfonyl)-2-methoxybenzamide (crude freebase) and 141 kg of isopropyl acetate were charged to a 400 L reactor. The mixture was heated to 60° C. and filtered. The reactor and filter were rinsed with 17.6 kg of isopropyl acetate and the combined isopropyl acetate mixture was concentrated to 98 L under reduced pressure. The temperature was adjusted to 50° C. and seeded with 0.67 kg of (S)-amisulpride of Form A′. The mixture was then stirred at 50° C. for 90 min and slowly cooled to 15° C. over 6 h, and the slurry was stirred 15° C. for 1 h and then isolated by filtration in a filter dryer. The mother liquors were recirculated to the reactor and filter dryer and the solid was washed with 35.2 kg of isopropyl acetate at 15° C. The isolated solid was transferred to a filter dryer and dried until dry (loss on drying<0.5%). This yielded 26.1 kg of (S)-4-Amino-N-[(1-ethyl-2-pyrrolidinyl)methyl]-5-(ethylsulfonyl)-2-methoxybenzamide of Form A′. Analysis by titration and analysis by mass balance indicated that the product was greater than about 99.8 wt % chemically pure. Chiral HPLC indicated that the product was greater than about 99.5% enantiomerically pure. XRPD characterization is shown in FIG. 6B and Table 8-1.

### Example 5: (R)-4-Amino-N-[(1-ethyl-2-pyrrolidinyl)methyl]-5-(ethylsulfonyl)-2-methoxybenzamide of Form A; Scheme 1B Scale-Up

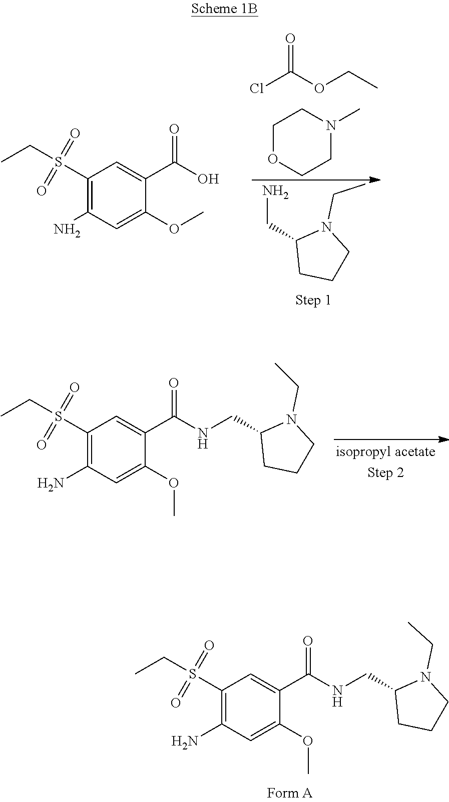

Example 5, Step 1: (R)-4-Amino-N-[(1-ethyl-2-pyrrolidinyl)methyl]-5-(ethylsulfonyl)-2-methoxybenzamide (crude freebase): First 29.7 kg of 4-amino-5-ethylsulfonyl)-2-methoxybenzoic acid and 158 kg of acetone were charged to a 400 L reactor. The mixture was cooled to −10° C. and 14 kg of ethyl chloroformate and 7 kg of acetone rinse was added to the mixture. Then 15 kg of 4-methylmorpholine and a 4 kg acetone rinse were slowly charged to the reactor (reaction is exothermic) while maintaining the temperature below 0° C. Then 15.78 kg of (R)-(1-ethylpyrrolidin-2-yl)methanamine was slowly added to the reactor while maintaining the temperature below 5° C. and the reaction was stirred at 5° C. for 1 h and then heated to 22° C. The mixture was stirred for another 11 h at 20° C. and concentrated under reduced pressure to 59 L. The temperature was then adjusted to 22° C. and solution of 26 wt % potassium carbonate (141 kg) and 149 kg isopropyl acetate was added to the mixture. The reaction mixture was stirred for 11 min, the stirring stopped, and the layers allowed to separate. The aqueous layer was removed and 59.5 kg of water added to the organic layer. The reaction mixture was stirred for 10 min, the stirring stopped, and the layers allowed to separate. The aqueous layer was removed, and 59.5 kg of water added to the organic layer. The reaction mixture was stirred for 10 min, the stirring stopped, and the layers allowed to separate. The aqueous layer was removed and the reactor was equipped with a Dean Stark apparatus. The solution was stirred at reflux with the Dean Stark apparatus removing water. The reactor temperature reached 91.8° C. as the water was removed, and the solution was concentrated to 100 L. The reaction temperature was reduced to 45° C. and the water content was measured and found to be 0.2% by Karl Fischer titration. The solution was stirred at 45° C. and seeded with 0.6 kg of (R)-amisulpride of Form A. The mixture was stirred for 30 min at 45° C. and the resulting slurry was cooled to 15° C. over 3.5 h. The slurry was stirred at 15° C. for 6.5 h and filtered. The mother liquors from the filtration were recirculated through the reactor and filter and the isolated solid was washed with 36 kg of isopropyl acetate at 15° C. The solid was dried in a vacuum oven at 35° C. to yield 33.3 kg of (R)-4-Amino-N-[(1-ethyl-2-pyrrolidinyl)methyl]-5-(ethylsulfonyl)-2-methoxybenzamide (crude freebase).

Example 5, Step 2 (recrystallization): (R)-4-Amino-N-[(1-ethyl-2-pyrrolidinyl)methyl]-5-(ethylsulfonyl)-2-methoxybenzamide substantially (Form A′): The 33.3 kg of (R)-4-Amino-N-[(1-ethyl-2-pyrrolidinyl)methyl]-5-(ethylsulfonyl)-2-methoxybenzamide (crude freebase) and 133 kg of isopropyl acetate were charged to a 400 L reactor. The mixture was heated to 60° C. and filtered. The reactor and filter were rinsed with 16.7 kg of isopropyl acetate and the combined isopropyl acetate mixture was concentrated to 93 L under reduced pressure at 50 to 60° C. The temperature was adjusted to 50° C. and seeded with 0.5 kg of (R)-amisulpride of Form A. The mixture was stirred at 50° C. for 90 min and slowly cooled to 15° C. over 6.5 h, and the slurry was stirred at 15° C. for 2 h and then isolated by filtration in a filter dryer. The mother liquors were recirculated to the reactor and filter dryer and the solid was washed with 33.5 kg of isopropyl acetate at 15° C. The isolated solid was transferred to a drying oven and dried under high vacuum until dry (loss on drying<0.5%). This yielded 24.4 kg of (R)-4-Amino-N-[(1-ethyl-2-pyrrolidinyl)methyl]-5-(ethylsulfonyl)-2-methoxybenzamide of Form A. Analysis by titration and analysis by mass balance indicated that the product was greater than about 99.8 wt % chemically pure. Chiral HPLC indicated that the product was greater than about 99.5% enantiomerically pure. XRPD characterization is shown in FIG. 6A and Table 8-1.

### Example 6: General Method of Making (S)-4-Amino-N-[(1-ethyl-2-pyrrolidinyl)methyl]-5-(ethylsulfonyl)-2-methoxybenzamide of Form A′ using a tertiary amine salt

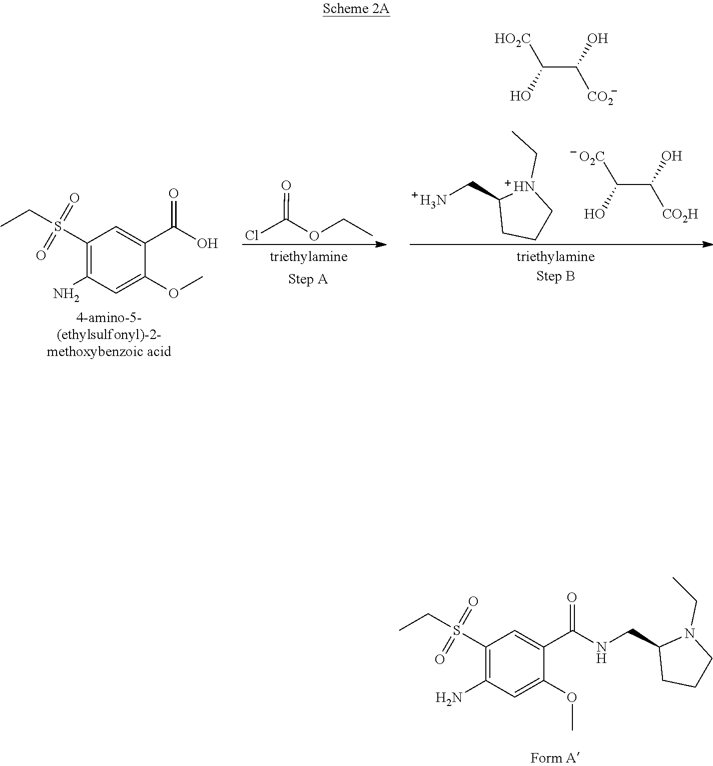

Without being held to theory, it is believed that 4-amino-5-(ethylsulfonyl)-2-methoxybenzoic acid reacts with ethyl chloroformate to form an anhydride intermediate (structure shown below) after Step A in Scheme 2A.

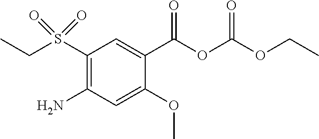 [SMILES: CCOC(=O)OC(=O)c1cc(S(=O)(=O)CC)c(N)cc1OC]

To a mixture of 4-amino-5-(ethanesulfonyl)-2-methoxybenzoic acid in acetone at −13° C. is added ethyl chloroformate, followed by triethylamine. The mixture is stirred at −10° C. for 1 h. Then (S)-1-ethylpyrrolidin-2-yl)methanamine bis D-tartrate salt, acetone, and triethylamine are added and the mixture is stirred for 1 h at 0° C. The mixture is then stirred at 20° C. for 16 h and concentrated under reduced pressure. The residue is then diluted with isopropyl acetate and aqueous potassium carbonate. The aqueous layer is removed and the organic layer is washed with water twice. The water in the resulting organic layer is removed via azeotropic distillation to give an organic solution with the water content below 0.5 wt %. This organic solution is concentrated to approximately 35 wt %. This mixture is heated to 45° C. and seeded with (S)-4-amino-N-[(1-ethyl-2-pyrrolidinyl)methyl]-5-(ethylsulfonyl)-2-methoxybenzamide of Form A′, stirred at 45° C. for 30 min, then cooled to 15° C. over 180 min. The resulting slurry is stirred at 15° C. for 1 h and filtered to give a solid. This solid is washed with cold isopropyl acetate and dried under vacuum to yield of (S)-4-amino-N-[(1-ethyl-2-pyrrolidinyl)methyl]-5-(ethylsulfonyl)-2-methoxybenzamide substantially of Form A′.

### Example 7: General Method of Making (R)-4-Amino-N-[(1-ethyl-2-pyrrolidinyl)methyl]-5-(ethylsulfonyl)-2-methoxybenzamide of Form A using a tertiary amine salt

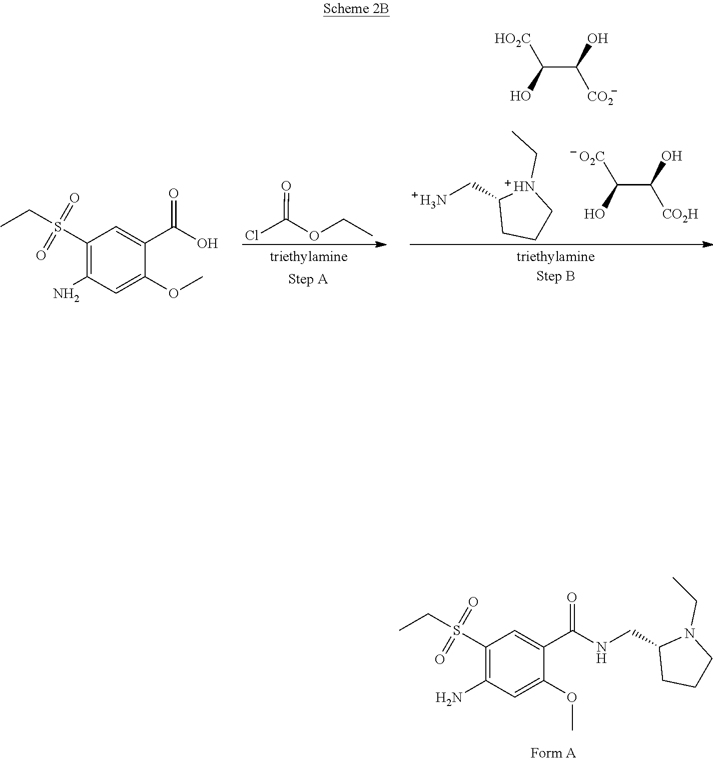

Without being held to theory, it is believed that an anhydride intermediate (structure shown below) is formed after Step A in Scheme 2B.

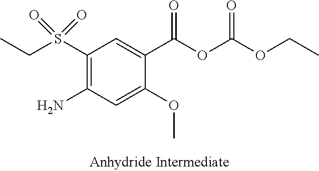 [SMILES: CCOC(=O)OC(=O)c1cc(S(=O)(=O)CC)c(N)cc1OC]

To a mixture of 4-amino-5-(ethanesulfonyl)-2-methoxybenzoic acid in acetone at −13±5° C., is added ethyl chloroformate, followed by triethylamine. The mixture is stirred at −10° C. for 1 h. Then (R)-(1-ethylpyrrolidin-2-yl)methanamine bis L-tartrate salt, acetone, and triethylamine are added and the mixture is stirred for 1 h at 0° C. The mixture is allowed to warm and stirred at 20° C. for 16 h and concentrated under reduced pressure. The residue is then diluted with isopropyl acetate and aqueous potassium carbonate. The aqueous layer is removed and the organic layer was washed with water twice. The water in the resulting organic layer is removed via azeotropic distillation to give an organic solution with the water content below 0.5 wt %. The organic solution is concentrated to give a mixture of about 35 wt % of the organic solution. The mixture is heated to 45° C. and seeded with about 1 wt % (R)-4-amino-N-[(1-ethyl-2-pyrrolidinyl)methyl]-5-(ethylsulfonyl)-2-methoxybenzamide of Form A, stirred at 45° C. for 30 min, then cooled to 15° C. over 180 min. The resulting slurry is stirred at 15° C. for 1 h, filtered to give a solid. This solid is washed with cold (15° C.) isopropyl acetate and dried under vacuum to yield of (R)-4-amino-N-[(1-ethyl-2-pyrrolidinyl)methyl]-5-(ethylsulfonyl)-2-methoxybenzamide substantially of Form A.

### Example 8: (R)-4-Amino-N-[(1-ethyl-2-pyrrolidinyl)methyl]-5-(ethylsulfonyl)-2-methoxybenzamide of Form A using a tertiary amine salt; Scheme 2B Lab Scale

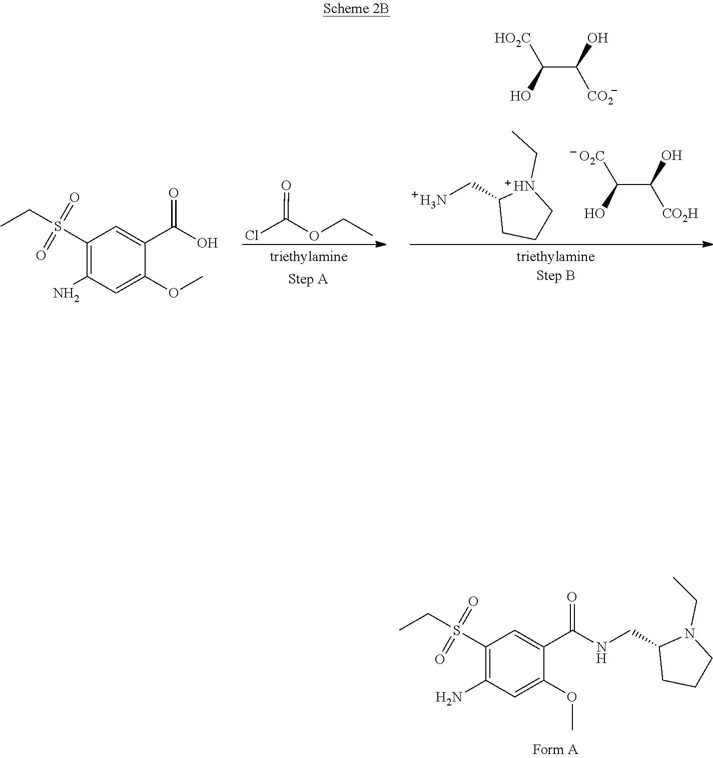

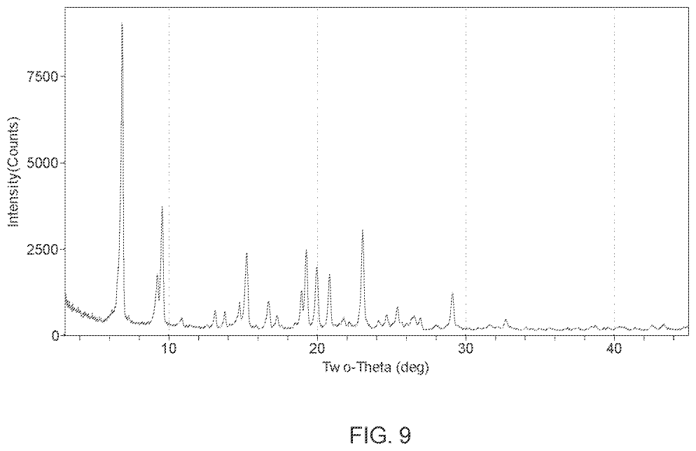

First 25 g of 4-amino-5-(ethanesulfonyl)-2-methoxybenzoic acid and 132 g of acetone were placed into a 500 mL flask. The mixture was cooled to −13° C. and 12 g of ethylchloroformate was added. Then 12.5 g of triethylamine was added and the mixture was stirred at −10° C. for 1 h. Then 44.2 g of (R)-(1-ethylpyrrolidin-2-yl)methanamine bis L-tartrate salt, 18 g of acetone, and 44 g of triethylamine were added and the mixture was stirred for 1 h at 0° C. The mixture was allowed to warm and stirred at 20° C. for 16 h and concentrated under reduced pressure. The residue was then diluted with 125 g of isopropyl acetate and 119 g of 26 wt % aqueous potassium carbonate. The mixture was stirred and the phases separated. The aqueous layer was removed and the organic layer was washed with 50 g of water twice. The organic layer was then dried by azeotropic drying via a Dean-Stark trap until the water separation substantially stops. The organic solution was then concentrated to yield a mixture of 35 wt % of the organic solution. The mixture was heated to 45° C. and seeded with 0.5 g of (R)-4-amino-N-[(1-ethyl-2-pyrrolidinyl)methyl]-5-(ethylsulfonyl)-2-methoxybenzamide of Form A. The mixture was then stirred at 45° C. for 30 min, then cooled to 15° C. over 180 min. The resulting slurry was then stirred at 15° C. for 1 h, filtered to give a solid. This solid is washed with 31 g of cooled (15° C.) isopropyl acetate and dried under vacuum to yield 23.9 g of (R)-4-amino-N-[(1-ethyl-2-pyrrolidinyl)methyl]-5-(ethylsulfonyl)-2-methoxybenzamide substantially of Form A. ¹H NMR (400 MHz, CHLOROFORM-d) δ ppm 1.11 (t, J=7.24 Hz, 3H) 1.24 (t, J=7.43 Hz, 3H) 1.55-1.77 (m, 3H) 1.79-1.91 (m, 1H) 2.13-2.27 (m, 2H) 2.51-2.71 (m, 1H) 2.73-2.94 (m, 1H) 3.07-3.28 (m, 4H) 3.68 (ddd, J=13.60, 7.34, 2.93 Hz, 1H) 3.93 (s, 3H) 5.55 (s, 2H) 6.22 (s, 1H) 8.05 (br d, J=4.70 Hz, 1H) 8.51 (s, 1H). ¹³C NMR (101 MHz, CHLOROFORM-d) δ ppm 7.24, 14.24, 22.91, 28.44, 41.33, 47.92, 49.66, 53.65, 56.02, 62.24, 98.47, 112.27, 112.77, 136.33, 150.57, 162.42, 164.09. XRPD characterization is shown in FIG. 9 and Table 8-2 in Example 9.

### Example 9: Characterization of Crystalline Amisulpride Enantiomers Made Substantially According to Example 4 and Example 5

This example presents characterization and other data on (S)-amisulpride of Form A′ made by the method described in Example 4, and on (R)-amisulpride of Form A made by the method described in Example 5.

Table 6 summarizes various properties and data on Form A′ crystalline form of (S)-amisulpride prepared from Example 4 and Form A crystalline form of (R)-amisulpride prepared from Example 5 made substantially by the methods of, respectively, Examples 1-5. The FIG. references in Table 6 are to figures in the present application.

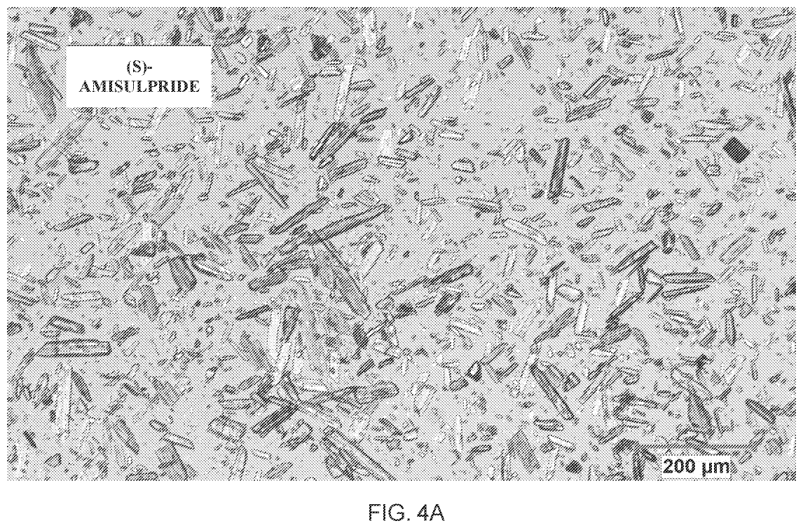

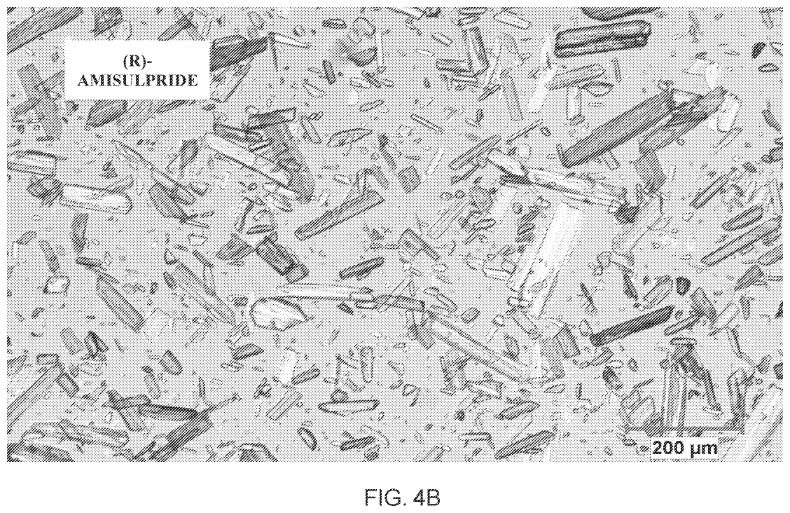

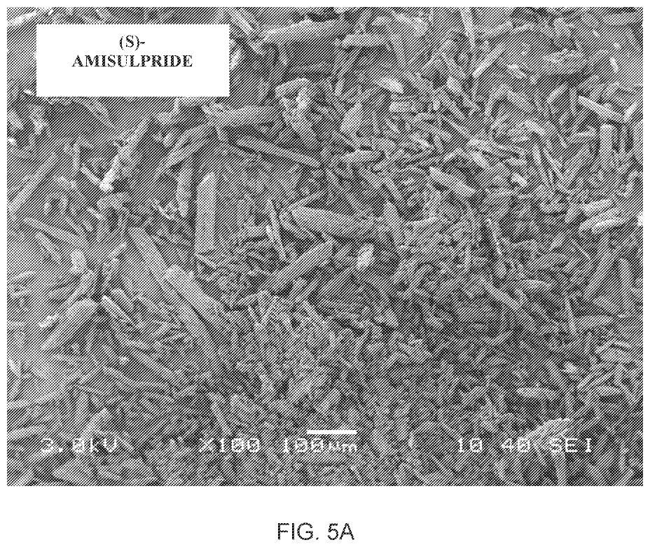

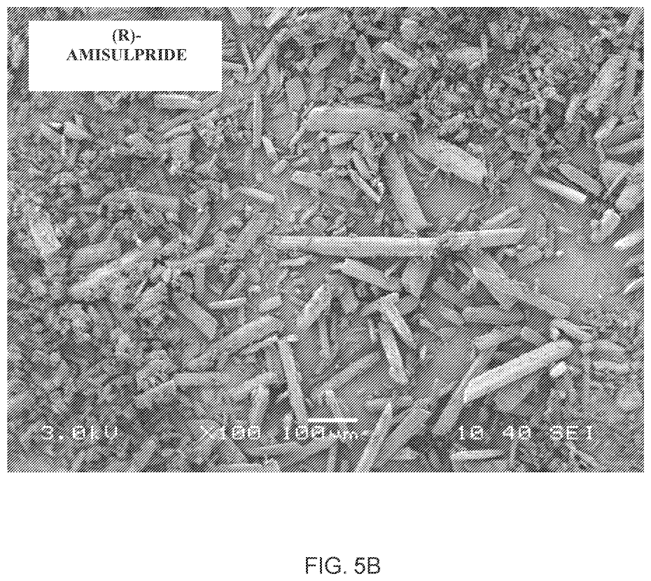

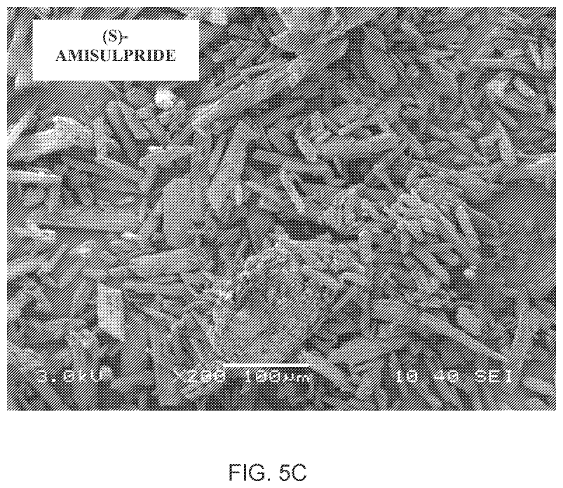

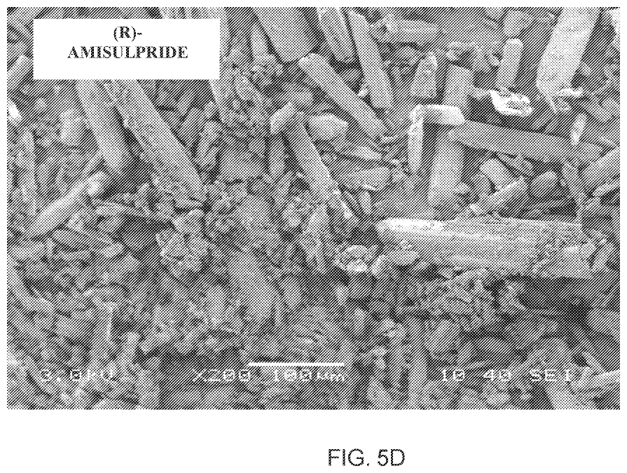

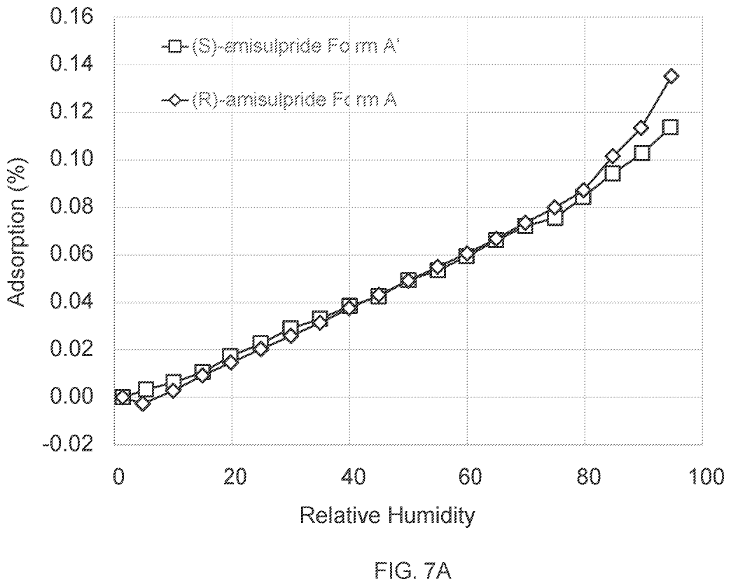

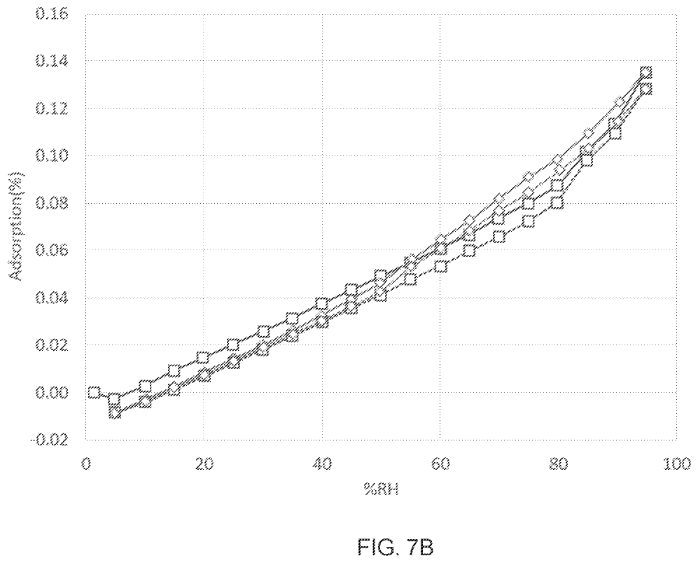

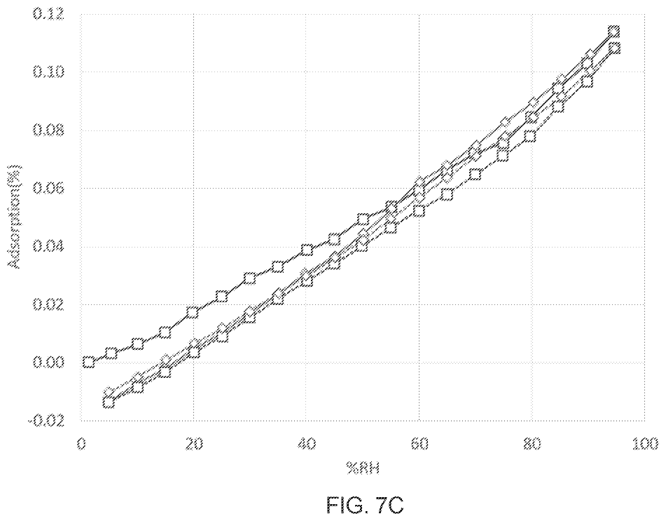

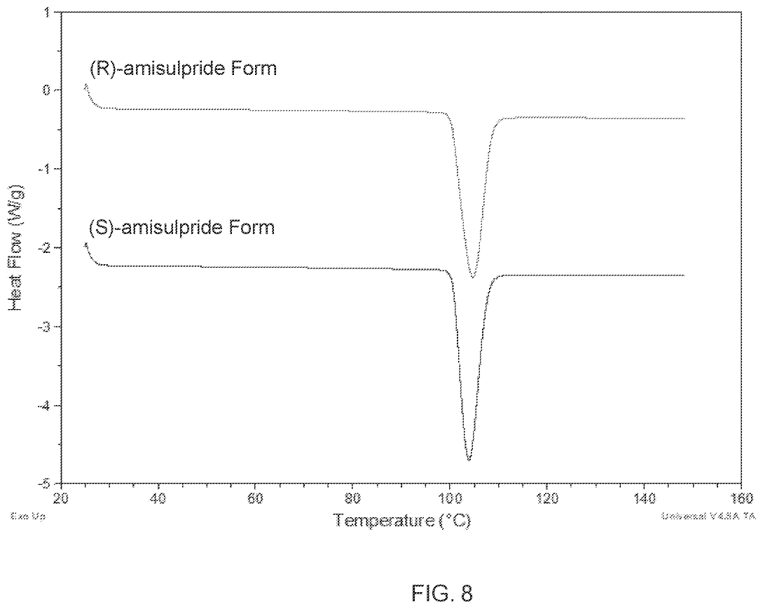

TABLE 6

| | (S)-amisulpride | (R)-amisulpride |
|---|---|---|
| Physical Property | Form A′ | Form A |
| PSD | FIG. 3 | FIG. 3 |
| Average PSD D10 (μm) | 9.5 | 14.3 |
| Average PSD D50 (μm) | 32.3 | 47.7 |
| Average PSD D90 (μm) | 116.9 | 133.9 |
| PLM Image | FIG. 4A | FIG. 4B |
| SEM Images | FIGS. 5A, 5C | FIGS. 5B, 5D |
| Crystal Habit | Needles/Rods | Needles/Rods |
| XRPD Pattern | FIG. 6B | FIG. 6A |
| DVS Isotherm | FIG. 7 | FIG. 7 |
| DVS Ads. at 95% RH | 0.09 | 0.14 |
| SSA (m²/g) | 0.65 | 0.51 |
| Melting Point, ° C. | 104 | 104 |
| DSC Thermogram | FIG. 8 | FIG. 8 |
| DSC Peak (° C.) ΔHf (J/g) | 103.9 (63) | 104.7 (64) |

Prior to analysis, the Form A′ crystals of (S)-amisulpride and Form A crystals of (R)-amisulpride were separately pre-sieved through a 30 mesh screen. All material was passed through screen during this pre-sieving step including aggregates.

Particle size distribution (PSD) measurements were measured by laser diffraction using a Malvern Instruments Mastersizer 3000 particle size analyzer instrument operated with the instrumentation and parameters described in Table 7.

TABLE 7
PSD Instrument and Data Collection Parameters

| Overhead Stirrer: | Impeller Stainless Steel Blade | |
|---|---|---|
| | (A100 Series) - 7.9 mm ( 5/16″) | |
| | bore diameter; 2.5 cm (1.0″) blade | |
| | diameter (LIGHTNIN ®) or similar | |
| | Stainless Steel Shaft - 30.5 cm (12″) | |
| | L; 9.5 mm (⅜″) | |
| | diameter shaft or similar | |
| Stirrer/Pump Speed: | 2100 | rpm |
| Ultrasound: | No Sonication | |
| Sample Refractive Index: | 1.5 (red and blue light) | |
| Sample Absorption: | 0.1 (red and blue light) | |
| Dispersant: | 0.2% Span 85 (Sorbitan | |
| | trioleate) in Hexanes | |
| Dispersant Refractive Index: | 1.38 | |
| Model: | General Purpose - Normal Sensitivity | |
| Sample Measurement Time: | 18 | seconds |
| Sample Measurement Snaps: | 18000 | |
| Background Measurement Time: | 18 | seconds |

| | |
|---|---|
| Background Measurement Snaps: | 18000 |
| Number of Measurement Cycles: | 1 |

The samples were first sieved through a 30 mesh screen prior to suspension. Approximately 400 mg of the sieved sample was weighed into a 50 ml beaker and 40 ml of a 0.2% v/v Span 85 (Sorbitan trioleate) in hexanes dispersant solution (e.g., a dispersant solution prepared by dissolving 2 mL of Span 85 (Sorbitan trioleate) in 1 L of hexanes) added. The overhead stirrer impeller was placed slightly above the bottom of the beaker (about 1 to 1.5 cm) and stirred at 500 rpm. After 1 minute, a disposable graduated transfer pipette was used to remove about 3 mL of the sample suspension from the center position of the beaker between impeller shaft and beaker side, and midway between top of vortex and beaker bottom. The entire contents of the pipette were added to the Malvern sample cell without inverting the pipette and the pipette rinsed twice with the circulating dispersant from the sample cell. The obscuration should be between 7% and 15%, if the obscuration was less than 7%, another aliquot of sample was added, making sure to transfer the entire aliquot taken up in the transfer pipette to the instrument, in order to avoid, e.g., potential artifacts due to settling of the suspension within the pipette. If the obscuration was greater than 15%, the instrument was drained and rinsed, and the measurement performed using a smaller sample suspension aliquot. After a sample cell with acceptable obscuration was obtained, the sample was circulated in the Mastersizer Hydro MV circulation system for two minutes and the sample dispersion measured.

Polarized light microscopy (PLM) measurements were made using a Nikon Microphot polarizing light microscope. Samples were prepared in Isopar G with 3% Lecithin and observed using cross-polarized light and imaged using cross-polarized light with a quarter wave plate.

Scanning electron microscope (SEM) measurements were conducted by Au sputter-coating samples using the Denton Desk II SEM coating system and the coated samples were imaged using the JEOL 6560 scanning electron microscope equipped with a tungsten filament at an accelerating voltage of 3 kV. The SEM showed small aggregates and fine particles coating the surfaces for both the (S)-amisulpride and (R)-amisulpride crystal batches.

XRPD analysis was performed using a Rigaku MiniFlex II Desktop X-Ray diffractometer using Cu radiation. The tube voltage and amperage were set to 30 kV and 15 mA, respectively. The scattering slit was fixed at 1.250 and the receiving slit was fixed at 0.3 mm. Diffracted radiation was detected by a NaI scintillation detector. A θ-2θ continuous scan at 1.0°/min with a step size of 0.02-0.05° from 3 to 45° 2θ was used. Data were collected and analyzed using Jade 8.5.4. Each sample was prepared for analysis by placing it in a low background, round, 0.1 mm indent sample holder.

Dynamic vapor sorption (DVS) isotherms were generated using a VTI SGA-100 Symmetric Vapor Sorption Analyzer. Analysis included pre-analysis drying at 25° C. with equilibrium criteria of 0.0000 wt % change in 5 minutes or a maximum of 180 minutes. Equilibrium criteria were the lesser of 0.01 wt % change in 5 minutes or 180 minutes at each RH step. Temperature was fixed at 25° C. and the relative humidity steps (25 to 95% to 25%) were made in 5% increments. Analysis was repeated for each sample in consecutive analyses (i.e., the sample was not removed from analyzer between analyses).

Specific surface area (SSA) was measured using a Quantachrome Quadrasorb SI surface area analyzer performed at six-points over the relative pressure range of 0.1000 to 0.3500 in 0.0500 increments. Approximately 1.3 to 4.6 grams of sample were used per analysis and were weighed into bulb tubes. Samples in bulb tubes were degassed 2 hours at 40° C. under vacuum using the Quantachrome FlowVac degasser prior to surface area analysis. Nitrogen gas was used as the gas for the surface area analyses. The surface area was found to be in the range 0.5-0.7 m²/g for the samples analyzed.

Differential scanning calorimetry (DSC) analyses was performed using TA Instruments Q100 differential scanning calorimeter. Samples were analyzed in an aluminum pan with crimped lid. Each sample was heated under a 50 mL/min nitrogen purge at a heating rate of 10° C./min, from a starting temperature of 25° C. up to a final temperature of 150° C.

TABLE 8-1
XRPD Peak List of FIGS. 6A and 6B

| FIG. 6A (R)-amisulpride Form A | | | FIG. 6B (S)-amisulpride Form A′ | | |
|---|---|---|---|---|---|
| Prepared from Example 5 | | | Prepared from Example 4 | | |
| | | Relative | | | Relative |
| 2-Theta | Height | Height % | 2-Theta | Height | Height % |
| 6.22 | 204 | 2 | 6.16 | 226 | 3.2 |
| 6.88 | 10256 | 100 | 6.86 | 7180 | 100 |
| 9.22 | 1517 | 14.8 | 9.2 | 1186 | 16.5 |
| 9.6 | 3353 | 32.7 | 9.56 | 3097 | 43.1 |
| 10.88 | 305 | 3 | 10.9 | 275 | 3.8 |
| 13.14 | 781 | 7.6 | 13.12 | 428 | 6 |
| 13.8 | 480 | 4.7 | 13.74 | 336 | 4.7 |
| 14.8 | 872 | 8.5 | 14.76 | 802 | 11.2 |
| 15.28 | 2739 | 26.7 | 15.26 | 2790 | 38.9 |
| 16.76 | 736 | 7.2 | 16.78 | 747 | 10.4 |
| 17.32 | 311 | 3 | 17.32 | 373 | 5.2 |
| 18.54 | 241 | 2.4 | 18.92 | 907 | 12.6 |
| 18.96 | 1739 | 17 | 19.28 | 2105 | 29.3 |
| 19.3 | 2567 | 25 | 20.02 | 1832 | 25.5 |
| 20.02 | 3159 | 30.8 | 20.84 | 1216 | 16.9 |
| 20.88 | 1976 | 19.3 | 21.75 | 203 | 2.8 |
| 21.78 | 543 | 5.3 | 23.1 | 2633 | 36.7 |
| 23.08 | 2629 | 25.6 | 24.2 | 249 | 3.5 |
| 24.16 | 192 | 1.9 | 24.66 | 253 | 3.5 |
| 24.68 | 327 | 3.2 | 25.38 | 507 | 7.1 |
| 25.42 | 792 | 7.7 | 26.58 | 400 | 5.6 |
| 26.56 | 595 | 5.8 | 26.98 | 314 | 4.4 |
| 26.92 | 483 | 4.7 | 28.02 | 106 | 1.5 |
| 28.02 | 173 | 1.7 | 29.1 | 975 | 13.6 |
| 29.14 | 1251 | 12.2 | 31.6 | 114 | 1.6 |
| 29.5 | 167 | 1.6 | 32.68 | 250 | 3.5 |
| 31.66 | 136 | 1.3 | 34.06 | 66 | 0.9 |
| 32.14 | 172 | 1.7 | 35.62 | 80 | 1.1 |
| 32.74 | 315 | 3.1 | 38.42 | 124 | 1.7 |
| 33.1 | 197 | 1.9 | 38.76 | 143 | 2 |
| 34.02 | 68 | 0.7 | 40.28 | 108 | 1.5 |
| 34.49 | 86 | 0.8 | 40.58 | 115 | 1.6 |
| 35.66 | 97 | 0.9 | 42.58 | 114 | 1.6 |
| 38.46 | 225 | 2.2 | 43.36 | 173 | 2.4 |
| 38.76 | 284 | 2.8 | | | |
| 39.36 | 92 | 0.9 | | | |
| 40.26 | 209 | 2 | | | |
| 40.66 | 110 | 1.1 | | | |
| 42.6 | 151 | 1.5 | | | |
| 43.4 | 238 | 2.3 | | | |

TABLE 8-2
XRPD Peak List of FIG. 9
FIG. 9. (R)-amisulpride Form A
Prepared from Example 8

| | | Relative |
|---|---|---|
| 2-Theta | Height | Height % |
| 6.16 | 306 | 3.5 |
| 6.84 | 8637 | 100.0 |
| 9.22 | 1461 | 16.9 |
| 9.54 | 3399 | 39.4 |
| 10.84 | 257 | 3.0 |
| 13.12 | 471 | 5.5 |
| 13.76 | 442 | 5.1 |
| 14.76 | 714 | 8.3 |
| 15.24 | 2206 | 25.5 |
| 15.86 | 123 | 1.4 |
| 16.68 | 777 | 9.0 |
| 17.28 | 355 | 4.1 |
| 18.94 | 1105 | 12.8 |
| 19.26 | 2300 | 26.6 |
| 19.96 | 1763 | 20.4 |
| 20.82 | 1495 | 17.3 |
| 21.78 | 244 | 2.8 |
| 23.04 | 2799 | 32.4 |
| 24.10 | 208 | 2.4 |
| 24.68 | 329 | 3.8 |
| 25.40 | 550 | 6.4 |
| 25.70 | 122 | 1.4 |
| 26.52 | 348 | 4.0 |
| 26.96 | 318 | 3.7 |
| 28.06 | 104 | 1.2 |
| 29.10 | 1041 | 12.1 |
| 31.64 | 113 | 1.3 |
| 32.70 | 264 | 3.1 |
| 34.06 | 92 | 1.1 |
| 35.62 | 73 | 0.8 |
| 37.60 | 67 | 0.8 |
| 38.48 | 106 | 1.2 |
| 38.72 | 133 | 1.5 |

TABLE 9A
DVS Water Sorption Isotherm (R)-Amisulpride Data of FIG. 7

| | | (R)-amisulpride |
|---|---|---|
| | | Form A prepared |
| | | from Example 5 |
| Relative | Elapse | Change Mass |
| Humidity % | time (min) | (wt %) |
| 1.39 | 155.38 | 0.000 |
| 4.79 | 327.39 | −0.003 |
| 10.00 | 340.39 | 0.003 |
| 14.96 | 359.89 | 0.009 |
| 19.81 | 366.91 | 0.015 |
| 24.91 | 374.93 | 0.020 |
| 30.02 | 382.94 | 0.026 |
| 34.96 | 390.96 | 0.031 |
| 39.94 | 398.96 | 0.038 |
| 44.99 | 406.96 | 0.043 |
| 49.99 | 415.98 | 0.049 |
| 54.96 | 424.99 | 0.055 |
| 59.96 | 434.01 | 0.061 |
| 64.91 | 443.03 | 0.067 |
| 69.86 | 452.04 | 0.073 |
| 74.86 | 461.04 | 0.080 |
| 79.80 | 470.04 | 0.087 |
| 84.74 | 479.04 | 0.101 |
| 89.58 | 488.06 | 0.113 |
| 94.72 | 497.06 | 0.135 |

TABLE 9B
DVS Water Sorption Isotherm (S)-Amisulpride Data of FIG. 7

| | | (S)-amisulpride |
|---|---|---|
| | | Form A′ prepared |
| | | from Example 4 |
| Relative | Elapse | Change Mass |
| Humidity % | time (min) | (wt %) |
| 1.41 | 180.73 | 0.000 |
| 5.38 | 195.14 | 0.003 |
| 10.12 | 207.64 | 0.006 |
| 15.00 | 219.64 | 0.011 |
| 19.73 | 227.64 | 0.017 |
| 24.91 | 235.64 | 0.023 |
| 29.95 | 243.64 | 0.029 |
| 34.98 | 251.64 | 0.033 |
| 40.01 | 260.64 | 0.038 |
| 44.94 | 269.64 | 0.042 |
| 50.00 | 278.64 | 0.049 |
| 55.02 | 287.66 | 0.053 |
| 59.89 | 296.66 | 0.059 |
| 64.92 | 305.66 | 0.066 |
| 69.86 | 314.66 | 0.072 |
| 74.97 | 323.66 | 0.076 |
| 79.71 | 332.66 | 0.084 |
| 84.74 | 341.66 | 0.094 |
| 89.75 | 350.66 | 0.103 |
| 94.55 | 359.68 | 0.114 |

Tables 9A and 9B presents the DVS water sorption for crystalline enantiomeric amisulpride of Form A and Form A′ shown in FIG. 7A. As can be seen, both crystalline (S)-amisulpride Form A′ and crystalline (R)-amisulpride Form A are substantially non-hygroscopic, exhibiting a maximum mass change of less than 0.15%.

### Example 10. Alternative General Procedure: (S)-4-Amino-N-[(1-ethyl-2-pyrrolidinyl)methyl]-5-(ethylsulfonyl)-2-methoxybenzamide substantially (Form A′)

Scheme 1A, Step 1: (S)-4-Amino-N-[(1-ethyl-2-pyrrolidinyl)methyl]-5-(ethylsulfonyl)-2-methoxybenzamide (crude freebase): To a mixture of 4-amino-5-(ethylsulfonyl)-2-methoxybenzoic acid in acetone at 20±5° C. was added ethyl chloroformate. The mixture was cooled. 4-Methylmorpholine was added followed by (S)-(1-ethyl pyrrolidin-2-yl)methanamine. The mixture was warmed to about 20-25° C., and after stirring for about 2 hours, the reaction mixture was concentrated and diluted with aqueous potassium carbonate and isopropyl acetate. The aqueous layer was removed and the organic layer was further diluted with isopropyl acetate. The organic layer was then washed with water twice. The isopropyl acetate solution (i.e., the organic layer) was dried via azeotropic distillation until the water content of the isopropyl acetate solution was below 0.3 wt %. The solution was concentrated to approximately 39 wt % followed by addition of methyl t-butyl ether. The temperature was adjusted to 45° C. The solution was seeded at 45° C. with (S)-4-Amino-N-[(1-ethyl-2-pyrrolidinyl)methyl]-5-(ethylsulfonyl)-2-methoxybenzamide in crystalline Form A′ and stirred at 45° C. for 45 minutes. The mixture was cooled to 15° C. and stirred for 1 h. The suspension was then filtered and the product cake was washed with isopropyl acetate and methyl t-butyl ether. The wet-cake was dried under vacuum at 40° C.±5° C. to a constant weight to yield (S)-4-amino-N-[(1-ethyl-2-pyrrolidinyl)methyl]-5-(ethylsulfonyl)-2-methoxybenzamide (crude freebase).

Scheme 1A, Step 2 (recrystallization): (S)-4-Amino-N-[(1-ethyl-2-pyrrolidinyl)methyl]-5-(ethylsulfonyl)-2-methoxybenzamide substantially (Form A′): Isopropyl acetate was added to (S)-4-Amino-N-[(1-ethyl-2-pyrrolidinyl)methyl]-5-(ethylsulfonyl)-2-methoxybenzamide (crude freebase from Step 1) and the mixture was heated to about 55° C. to achieve dissolution. The resulting solution was then passed through a filter. The filtrate was concentrated by distillation followed by methyl t-butyl ether addition. The resulting solution was cooled to 50° C. and seeded with Form A′. The resulting slurry was stirred at 50° C. for 1 hour and then slowly cooled to 15° C. The suspension was then filtered and the product cake was washed with isopropyl acetate and methyl t-butyl ether. The wet-cake was dried under vacuum at 35° C.±5° C. to constant weight to yield (S)-4-amino-N-[(1-ethyl-2-pyrrolidinyl)methyl]-5-(ethylsulfonyl)-2-methoxybenzamide, substantially as Form A′.

### Example 11. Alternative Lab Scale Procedure: (S)-4-Amino-N-[(1-ethyl-2-pyrrolidinyl)methyl]-5-(ethylsulfonyl)-2-methoxybenzamide substantially (Form A′)

Scheme 1A, Step 1: (S)-4-Amino-N-[(1-ethyl-2-pyrrolidinyl)methyl]-5-(ethylsulfonyl)-2-methoxybenzamide (crude freebase): To a mixture of 55 g of 4-amino-5-(ethylsulfonyl)-2-methoxybenzoic acid in 292 g of acetone at 20±5° C. was added 24 g of ethyl chloroformate. The mixture was cooled to 0° C. and 27.5 g of 4-methylmorpholine was added at <25° C. The mixture was cooled to 10° C. and 28.6 g of (S)-(1-ethyl pyrrolidin-2-yl)methanamine was added. The reaction mixture was warmed to 20-25° C. and stirred for 2 hours. Subsequently, the reaction mixture was concentrated to approximately 110 mL. The mixture was diluted with 233 g of 29 wt % aqueous potassium carbonate and 165 g of isopropyl acetate. The mixture was stirred for 10 min and the aqueous layer was removed. 110 g of DI water was added to the organic along with 138 g of isopropyl acetate. The mixture was stirred for 10 min and the aqueous layer was removed. 110 g of DI water was added to the organic layer, the mixture was stirred for 10 min, and the aqueous layer was removed. The isopropyl acetate solution (i.e., the organic layer) was dried via azeotropic distillation. The isopropyl acetate solution was further concentrated until the water content was below 0.3% by weight. 27.5 grams of methyl t-butyl ether was added and the system was prepared for seeding. The solution was seeded at 45° C. with (S)-4-Amino-N-[(1-ethyl-2-pyrrolidinyl)methyl]-5-(ethylsulfonyl)-2-methoxybenzamide of Form A′ and stirred at 45° C. for 45 min. The mixture was slowly cooled to 15° C. and stirred for 1 h. The suspension was then filtered and the product cake was washed with 44 g isopropyl acetate and 44 g of methyl t-butyl ether. The wet-cake was dried under vacuum at 40° C.±5° C. to constant weight to yield approximately 62 g of (S)-4-Amino-N-[(1-ethyl-2-pyrrolidinyl)methyl]-5-(ethylsulfonyl)-2-methoxybenzamide of Form A′ (crude freebase).

Scheme 1A, Step 2 (recrystallization): (S)-4-Amino-N-[(1-ethyl-2-pyrrolidinyl)methyl]-5-(ethylsulfonyl)-2-methoxybenzamide substantially (Form A′): Isopropyl acetate (242 g) was added to 60.5 g of (S)-4-Amino-N-[(1-ethyl-2-pyrrolidinyl)methyl]-5-(ethylsulfonyl)-2-methoxybenzamide and the mixture was heated to 55° C. to achieve dissolution. The resulting solution was then passed through a filter and rinsed with 34 g of isopropyl acetate. The filtrate was concentrated by distillation to about a 39 wt % solution and 24 g of methyl t-butyl ether were added. The resulting solution was adjusted to 50° C. and seeded with 0.45 g of (S)-4-Amino-N-[(1-ethyl-2-pyrrolidinyl)methyl]-5-(ethylsulfonyl)-2-methoxybenzamide substantially of Form A′, and the mixture was stirred for 1 h. The mixture was cooled to about 38° C. at a rate of about −0.1° C./min. Then, the mixture was cooled to about 10-15° C. at a rate of about −0.5° C./min. The resulting slurry was stirred at 10-15° C. for 1 h. Then, the resulting slurry was filtered and the product cake was washed with 61 g of isopropyl acetate and 25 g of methyl t-butyl ether. The wet-cake was dried under vacuum at 35° C.±5° C. to constant weight to yield 54.6 g of (S)-4-Amino-N-[(1-ethyl-2-pyrrolidinyl)methyl]-5-(ethylsulfonyl)-2-methoxybenzamide substantially as Form A′.

### Example 12. Alternative General Procedure: (R)-4-Amino-N-[(1-ethyl-2-pyrrolidinyl)methyl]-5-(ethylsulfonyl)-2-methoxybenzamide substantially (Form A)

Scheme 1A, Step 1: (R)-4-Amino-N-[(1-ethyl-2-pyrrolidinyl)methyl]-5-(ethylsulfonyl)-2-methoxybenzamide (crude freebase): To a mixture of 4-amino-5-(ethylsulfonyl)-2-methoxybenzoic acid in acetone at 20±5° C. was added ethyl chloroformate. The mixture was cooled. 4-Methylmorpholine was added followed by (R)-(1-ethyl pyrrolidin-2-yl)methanamine. The mixture was warmed to about 20-25° C. and stirred for about 2 h, then the reaction mixture was concentrated and diluted with aqueous potassium carbonate and isopropyl acetate. The aqueous layer was removed and the organic layer was further diluted with isopropyl acetate and washed with water twice. The water was removed via azeotropic distillation (until the water content of the isopropyl acetate solution was below about 0.3 wt %). The solution was concentrated to approximately 39 wt % followed by addition of methyl t-butyl ether. The temperature was adjusted to 45° C. The solution was seeded at 45° C. with about 1.5 wt % (R)-4-Amino-N-[(1-ethyl-2-pyrrolidinyl)methyl]-5-(ethylsulfonyl)-2-methoxybenzamide of Form A and stirred at 45° C. for 45 minutes. The mixture was cooled to 15° C. and stirred for 1 h. The suspension was then filtered and the product cake was washed with isopropyl acetate and methyl t-butyl ether. The wet-cake was dried under vacuum at 40° C.±5° C. to constant weight to yield (R)-4-amino-N-[(1-ethyl-2-pyrrolidinyl)methyl]-5-(ethylsulfonyl)-2-methoxybenzamide of Form A (crude freebase).

Scheme 1A, Step 2 (recrystallization): (R)-4-Amino-N-[(1-ethyl-2-pyrrolidinyl)methyl]-5-(ethylsulfonyl)-2-methoxybenzamide substantially (Form A): Isopropyl acetate was added to (R)-4-Amino-N-[(1-ethyl-2-pyrrolidinyl)methyl]-5-(ethylsulfonyl)-2-methoxybenzamide (crude freebase) and the mixture was heated to 55° C. to achieve dissolution. The resulting solution was then passed through a filter. The filtrate was concentrated by distillation followed by methyl t-butyl ether addition. The resulting solution was cooled to 50° C. and seeded with Form A. The resulting slurry was stirred at 50° C. for 1 hour and then slowly cooled to 15° C. The suspension was then filtered and the product cake was washed with isopropyl acetate and methyl t-butyl ether. The wet-cake was dried under vacuum at 35° C.±5° C. to constant weight to yield (R)-4-amino-N-[(1-ethyl-2-pyrrolidinyl)methyl]-5-(ethylsulfonyl)-2-methoxybenzamide substantially of Form A.

### Example 13. Alternative Lab Scale Procedure: (R)-4-Amino-N-[(1-ethyl-2-pyrrolidinyl)methyl]-5-(ethylsulfonyl)-2-methoxybenzamide of Form A

Scheme 1B, Step 1: (R)-4-Amino-N-[(1-ethyl-2-pyrrolidinyl)methyl]-5-(ethylsulfonyl)-2-methoxybenzamide (crude freebase): To a mixture of 75 g of 4-amino-5-(ethylsulfonyl)-2-methoxybenzoic acid in 393 g of acetone at about 20° C. was added 33 g of ethyl chloroformate. The mixture was cooled to about 0° C. and 37.5 g of 4-methylmorpholine was added at less than 25° C. The mixture was cooled to about 5° C. and 39 g of (R)-(1-ethyl pyrrolidin-2-yl)methanamine was added. The reaction mixture was warmed to about 20-25° C. and stirred for 2 hours. Subsequently, the reaction mixture was concentrated to approximately 150 mL. The mixture was diluted with 315 g of 29 wt % aqueous potassium carbonate and 225 g of isopropyl acetate. The mixture was stirred for 10 min and the aqueous layer was removed. 150 g of DI water was added to the organic along with 188 g of isopropyl acetate. The mixture was stirred for 10 min and the aqueous layer was removed. 150 g of DI water was added to the organic layer and the mixture was stirred for 10 min. The aqueous layer was removed. The isopropyl acetate solution (i.e., the organic layer) was dried via azeotropic distillation. The isopropyl acetate solution was further concentrated until the water content was below 0.3% by weight. Methyl t-butyl ether (38 g) was added and the system was prepared for seeding. The solution was seeded at about 45° C. with (R)-4-Amino-N-[(1-ethyl-2-pyrrolidinyl)methyl]-5-(ethylsulfonyl)-2-methoxybenzamide of Form A and stirred at about 45° C. for 45 min. The mixture was slowly cooled to about 15° C. and stirred for 1 h. The suspension was then filtered and the product cake was washed with 60 g isopropyl acetate and 60 g of methyl t-butyl ether. The wet-cake was dried under vacuum at about 40° C. to constant weight to yield approximately 85.5 g of (R)-4-Amino-N-[(1-ethyl-2-pyrrolidinyl)methyl]-5-(ethylsulfonyl)-2-methoxybenzamide of Form A (crude freebase).

Scheme 1B, Step 2 (recrystallization): (R)-4-Amino-N-[(1-ethyl-2-pyrrolidinyl)methyl]-5-(ethylsulfonyl)-2-methoxybenzamide substantially (Form A): Isopropyl acetate (337 g) was added to 83.6 g of (R)-4-Amino-N-[(1-ethyl-2-pyrrolidinyl)methyl]-5-(ethylsulfonyl)-2-methoxybenzamide and the mixture was heated to about 55° C. to achieve dissolution. The resulting solution was then passed through a filter and rinsed with approximately 42 g of isopropyl acetate. The filtrate was concentrated by distillation to about 39 wt % solution and methyl t-butyl ether (33 g) was added. The resulting solution was adjusted to about 50° C. and seeded with (R)-4-Amino-N-[(1-ethyl-2-pyrrolidinyl)methyl]-5-(ethylsulfonyl)-2-methoxybenzamide substantially of Form A, and the mixture was stirred for 1 h. The resulting slurry was cooled to about 15° C. and stirred for 1 h at this temperature. The suspension was then filtered and the product cake was washed with isopropyl acetate (84 g) and 34 g methyl t-butyl ether (34 g). The wet-cake was dried under vacuum at about 35° C. to constant weight to yield 76.8 g of (R)-4-Amino-N-[(1-ethyl-2-pyrrolidinyl)methyl]-5-(ethylsulfonyl)-2-methoxybenzamide substantially as Form A.

### Example 14. Alternative Procedure: (S)-4-Amino-N-[(1-ethyl-2-pyrrolidinyl)methyl]-5-(ethylsulfonyl)-2-methoxybenzamide of Form A′; Scheme 1A (Scaled-Up)

Scheme 1A, Step 1: (S)-4-Amino-N-[(1-ethyl-2-pyrrolidinyl)methyl]-5-(ethylsulfonyl)-2-methoxybenzamide (crude freebase): 200 kg of 4-amino-5-ethylsulfonyl-2-methoxybenzoic acid and 1,060 kg of acetone were charged to a 750 gallon reactor. While mixing at 20° C., 88 kg of ethyl chloroformate and 7 kg of acetone rinse was added to the mixture. After cooling to 0° C., 100 kg of 4-methylmorpholine and a 7 kg acetone rinse was slowly charged to the reactor while maintaining the temperature below 25° C. After cooling to 10° C., 104 kg of (S)-(1-ethylpyrrolidin-2-yl)methanamine was added and the reaction was stirred at 22° C. for 8 h. The mixture was concentrated under reduced pressure to about 400 L. The temperature was then adjusted to 25° C. and a solution of 29 wt % potassium carbonate (848 kg) and 600 kg isopropyl acetate was added to the mixture. The phases were mixed, the stirring stopped, and the layers were allowed to separate. The aqueous layer was removed and 400 kg of water along with 500 kg of isopropyl acetate were added to the organic layer. The reaction mixture was mixed, the stirring stopped, and the layers allowed to separate. The aqueous layer was removed and 400 kg of water added to the organic layer. The reaction mixture was stirred for 10 min, the stirring stopped, and the layers were allowed to separate. The aqueous layer was removed and the reactor was equipped with a Dean-Stark apparatus. The solution was then stirred at reflux with the Dean Stark apparatus removing water. After the water content was reduced, the mixture was then concentrated to not less than 40 wt %. The water content was measured and found to be not more than 0.3% by Karl Fischer titration. The system was diluted with isopropyl acetate as required, then the temperature was adjusted to 55° C. and methyl t-butyl ether (100 kg) was added. The temperature was adjusted to 45° C. and seeded with 4 kg of (S)-amisulpride of Form A′. The mixture was stirred for 45 min at 45° C. and the resulting slurry was cooled to 15° C. over 2.5 h. The slurry was then stirred at 15° C. for 3 h and filtered. The isolated solid was washed with 160 kg of isopropyl acetate and 160 kg of methyl t-butyl ether at 15° C. The solid was dried in a filter dryer at 40° C. to yield 230 kg of (S)-4-Amino-N-[(1-ethyl-2-pyrrolidinyl)methyl]-5-(ethylsulfonyl)-2-methoxybenzamide of Form A′ (crude freebase).

Scheme 1A, Step 2 (recrystallization): (S)-4-Amino-N-[(1-ethyl-2-pyrrolidinyl)methyl]-5-(ethylsulfonyl)-2-methoxybenzamide substantially (Form A′): 230 kg of (S)-4-Amino-N-[(1-ethyl-2-pyrrolidinyl)methyl]-5-(ethylsulfonyl)-2-methoxybenzamide (crude freebase) and 922 kg of isopropyl acetate were charged to a 750 gal reactor. The mixture was heated to NLT 55° C. and filtered. The reactor and filter were rinsed with 115 kg of isopropyl acetate and the combined isopropyl acetate mixture was concentrated to approximately 600 L under reduced pressure. The temperature was adjusted to 55° C. and 95 kg of methyl t-butyl ether was added. The mixture was cooled to 50° C. and seeded with 3.5 kg of (S)-amisulpride of Form A′. The mixture was then stirred at 50° C. for 90 min. The mixture was cooled to about 38° C. at a rate of about −0.1° C./min. Then, the mixture was cooled to about 10-15° C. at a rate of about −0.5° C./min. The resulting slurry was stirred at 10-15° C. for 1 h and then isolated by filtration in a filter dryer. The mother liquors were recirculated to the reactor and filter dryer and the solid was washed with 230 kg of isopropyl acetate and 94 kg of methyl t-butyl ether at 10-15° C. The isolated solid was dried until loss on drying less than 0.5%. This yielded 207.5 kg of (S)-4-Amino-N-[(1-ethyl-2-pyrrolidinyl)methyl]-5-(ethylsulfonyl)-2-methoxybenzamide, Form A′. Analysis by titration and analysis by mass balance indicated that the product was greater than about 99.8 wt % chemically pure. Chiral HPLC indicated that the product was greater than about 99.5% enantiomerically pure. XRPD characterization was consistent with Form A′.

### Example 15. Alternative Scaled-Up Procedure: (R)-4-Amino-N-[(1-ethyl-2-pyrrolidinyl)methyl]-5-(ethylsulfonyl)-2-methoxybenzamide of Form A; Scheme 1B (Scaled-Up)

(R)-4-Amino-N-[(1-ethyl-2-pyrrolidinyl)methyl]-5-(ethylsulfonyl)-2-methoxybenzamide Form A can also be prepared according in a method analogous to the alternative procedure of Example 14, using (R)-(1-ethylpyrrolidin-2-yl)methanamine in place of (S)-(1-ethylpyrrolidin-2-yl)methanamine.

### Example 16. Formulations of Form A′ and Form a Prepared by the Procedure of Example 14

The particle size distribution (PSD) of crystalline products prepared by the foregoing examples were examined. FIG. 10 presents a plot of the cooling profile comparison between the cooling profile X, using the recrystallization procedure of, e.g., Example 14, with cooling profile Y, using the recrystallization procedure of, e.g., Example 4 (Step 2). Recrystallization using the cooling methods of Example 14 (cooling method X) provided Form A′ having a smaller particle size distribution (e.g., D₅₀ and D₉₀ values) than Form A′ prepared by the methods of Example 4 (cooling method Y), as shown in the table below.

| | Form A′ | |
|---|---|---|
| | (averages of lots | |
| | prepared by the | Form A′ |
| | methods of | prepared by |
| Physical Property | Example 4 | Example 14 |
| PSD D₁₀ (μm) | 6.0 | 7.0 |
| PSD D₅₀ (μm) | 29.1 | 25.5 |
| PSD D₉₀ (μm) | 99.4 | 83.9 |

PSD data was obtained using a Malvern Instruments Mastersizer particle size analyzer instrument. PSD Instrument and data collection parameters are provided below.

PSD Instrument and Data Collection Parameters

| Overhead Stirrer: | Impeller Stainless Steel Blade | |
|---|---|---|
| | (A100 Series) - 7.9 mm ( 5/16″) | |
| | bore diameter; 2.5 cm (1.0″) | |
| | blade diameter (LIGHTNIN) | |
| | or similarStainless Steel Shaft - | |
| | 30.5 cm (12″) L; | |
| | 9.5 mm (⅜″) | |
| | diameter shaft or similar | |
| Stirrer/Pump Speed: | 2100 | rpm |
| Ultrasound: | No Sonication | |
| Sample Refractive Index: | 1.5 (red and blue light) | |
| Sample Absorption: | 1 (red and blue light) | |
| Dispersant: | 0.2% Span 85 (Sorbitan | |
| | trioleate) in Hexanes | |
| Dispersant Refractive Index: | 1.38 | |
| Model: | General Purpose - Normal Sensitivity | |
| Sample Measurement Time: | 12 | seconds |
| Sample Measurement Snaps: | 12000 | |
| Background Measurement Time: | 12 | seconds |

| | |
|---|---|
| Background Measurement Snaps: | 12000 |
| Number of Measurement Cycles: | 1 |

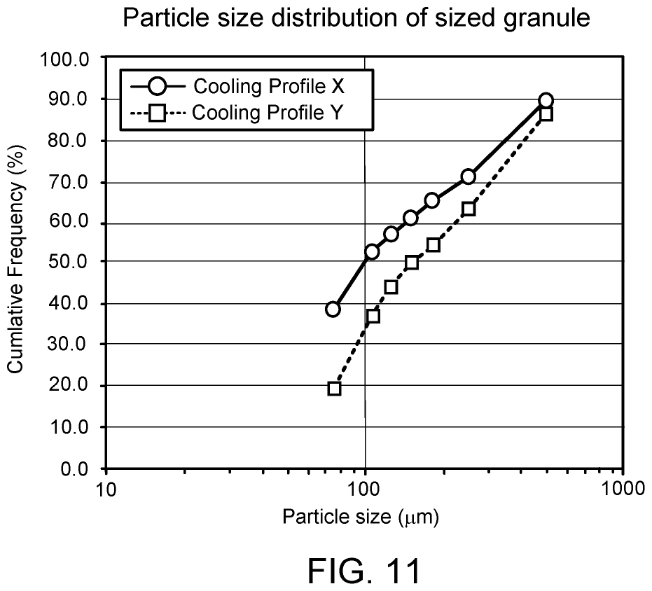

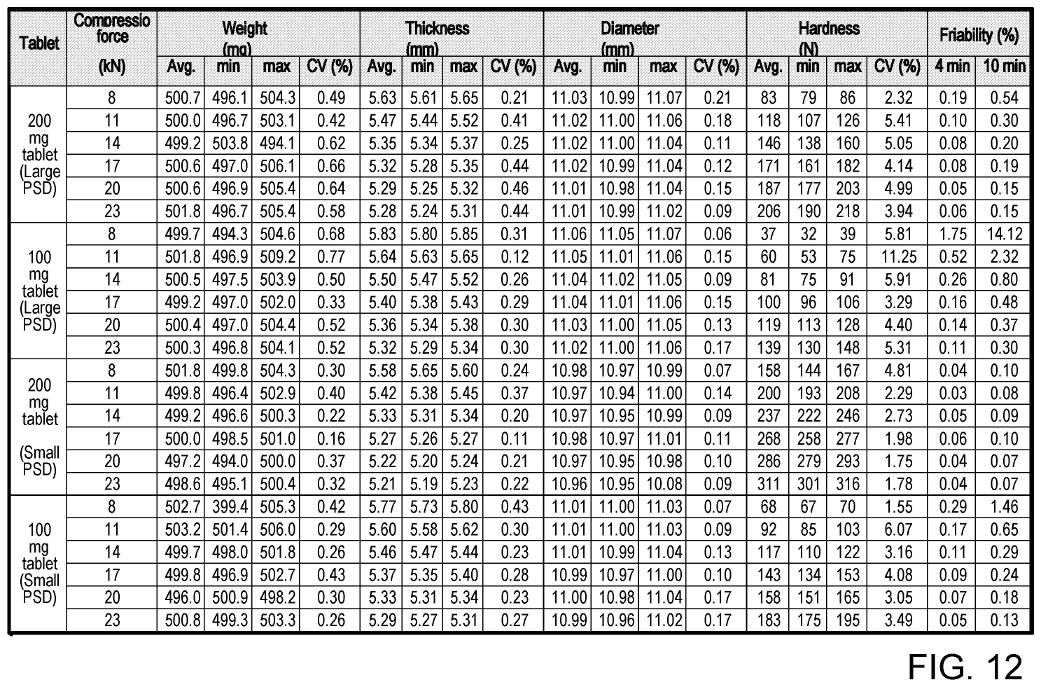

Crystalline (S)-amisulpride prepared using the method of Example 14, using cooling profile X, provided crystalline products having narrower and smaller particle size distributions (PSD). In the tablet manufacturing process, it was found that the granulation speed for crystalline amisulpride with larger PSD was significantly faster than that with smaller PSD. Spray was used to add a binder solution during the granulation process. However, faster granulation speeds increased the concentration of the polyvinyl alcohol binder solution used in the formulation. FIG. 11 shows a plot of the cumulative frequency versus particle size. If crystalline material having higher PSD was used, then it was necessary to use higher concentration of polyvinyl alcohol solution in the granulation process. Using crystalline amisulpride with smaller PSD allowed for a lower concentration of the polyvinyl alcohol binder, which allowed for more control in the tablet manufacturing process compared to crystalline amisulpride having larger PSD.

In addition, it was found that crystalline products having smaller PSD have higher hardness values, compared to tablets containing crystalline products having larger PSD. Controlled release tablets comprising (R)- and (S)-amisulpride can be prepared according to the processes provided in U.S. Pat. No. 11,160,758.

The PSD of the crystalline amisulpride used in the various formulations are provided below. Amisulpride corresponding to “small PSD” can be prepared using the recrystallization and cooling methods of, e.g., Examples 14 and 15 (i.e., cooling profile X of FIG. 10). Amisulpride corresponding to “large PSD” can be prepared using the recrystallization methods of Examples 1 and 3 (i.e., cooling profile Y of FIG. 10). The PSDs were measured using a Mastersizer 2000 particle size analyzer instrument as described above.

| Amisulpride Material | D₁₀ (μm) | D₅₀ (μm) | D₉₀ (μm) |
|---|---|---|---|
| “Form A small PSD” | 4.7 | 18.2 | 90.4 |
| “Form A large PSD” | 12.8 | 94.9 | 441.4 |
| “Form A′ small PSD” | 5.1 | 18.3 | 80.1 |
| “Form A′ large PSD” | 31.6 | 93.1 | 425.4 |

The details ofthe tablet compositions are provided below.

| | 200 mg tablet | | 100 mg tablet | | 200 mg tablet | | 100 mg tablet | |
|---|---|---|---|---|---|---|---|---|
| Component | (large PSD) | | (large PSD) | | (small PSD) | | (small PSD) | |
| “Form A large PSD” | 170 | mg | 85 | mg | — | | — | |
| “Form A small PSD” | — | | — | | 170 | mg | 85 | mg |
| “Form A′ large PSD” | 30 | mg | 15 | mg | — | | — | |
| “Form A′ small PSD” | — | | — | | 30 | mg | 15 | mg |
| D-mannitol | 29.5 | mg | 14.75 | mg | 29.5 | mg | 14.75 | mg |
| Partly pregelatinized | 29.5 | mg | 14.75 | mg | 29.5 | mg | 14.75 | mg |
| starch | | | | | | | | |
| Partially hydrolyzed | 8 | mg | 4 | mg | 8 | mg | 4 | mg |
| polyvinyl alcohol*¹ | | | | | | | | |
| Purified water*² | q.s | | q.s | | q.s | | q.s | |
| Granule total: | 267 | mg | 133.5 | mg | 267 | mg | 133.5 | mg |
| Hypromellose*³ | 125 | mg | 125 | mg | 125 | mg | 125 | mg |
| D-mannitol | 95.5 | mg | 114 | mg | 95.5 | mg | 114 | mg |
| Partly pregelatinized | — | | 115 | mg | — | | 115 | mg |
| starch | | | | | | | | |
| Light anhydrous silicic | 2.5 | mg | 2.5 | mg | 2.5 | mg | 2.5 | mg |
| acid | | | | | | | | |
| Sodium stearyl | 10 | mg | 10 | mg | 10 | mg | 10 | mg |
| fumarate | | | | | | | | |
| Core tablet total: | 500 | mg | 500 | mg | 500 | mg | 500 | mg |

q.s: quantum sufficiat
*¹Partial saponification (saponification value: 85.0-89.0 mol %, viscosity: 4.6-6.0 mPa · s)
*²It is not included in the total because it was removed in the drying process.
*³Substitution Type: 2208, Viscosity: 100 mPa · s

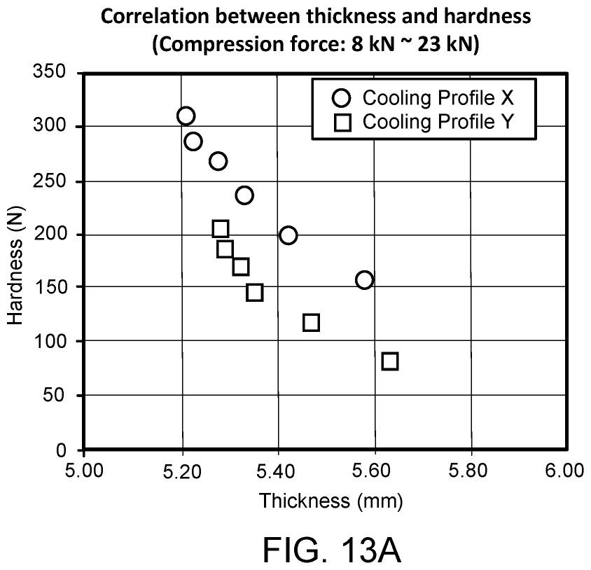

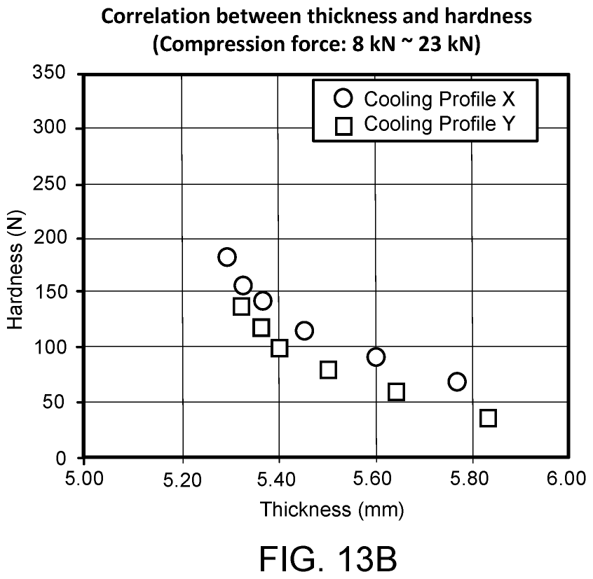

A summary of the physical properties of the core tablets is provided in FIG. 12. A plot of the correlation between tablet thickness and hardness of the 200 mg tablets is provided in FIG. 13A. A plot of the correlation between tablet thickness and hardness of the 100 mg tablets is provided in FIG. 13B.

In addition, Impurity AA of the following formula may be observed in crystalline (S)-amisulpride Form A′ prepared from Example 2 or 4, and/or crystalline (R)-amisulpride Form A prepared from Example 5 or 8:

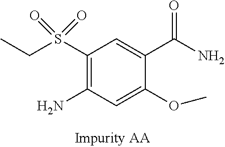 [SMILES: CCS(=O)(=O)c1cc(C(N)=O)c(OC)cc1N]

¹H NMR (400 MHz, DMSO-d) δ ppm 1.07 (t, J=7.24 Hz, 3H) 3.13 (q, J=7.30 Hz, 2H) 3.30 (s, 3H) 6.48 (s, 3H) 7.37 (br d, J=9.00 Hz, 2H) 8.10 (s, 1H); ¹³C NMR (100 MHz, DMSO-d) δ ppm 7.57, 48.78, 56.43, 98.48, 111.00, 111.10, 135.70, 151.94, 162.63, 165.11. Impurity AA may also be observed in crystalline (S)-amisulpride Form A′ prepared from Example 10, 11, 14, 15, or 16, and/or crystalline (R)-amisulpride Form A prepared from Example 5, 8, 12, 13, or 15. It may be found as a degradant in any of the products prepared from Examples 2, 4, 5, and 8 after a certain period of time (e.g., over at least one month at room temperature or an elevated temperature, or 0.01 wt % to 0.03 wt % over 6 months at 40° C., 75% relative humidity). Likewise, Impurity AA may be found as a degradant in any of the products prepared from Examples 10-16. In certain embodiments, Impurity AA may be present in about 0.001 wt % to about 0.3 wt % of any of the products prepared from Examples 2, 4, 5, and 8. In certain embodiments, Impurity AA may be present in about 0.01 wt % to about 0.2 wt % of any of the products prepared from Examples 2, 4, 5, and 8. In certain embodiments, Impurity AA may be present in about 0.001 wt % to about 0.3 wt % (e.g. 0.01 wt % to about 0.2 wt %) of any of the products prepared from Examples 2, 4, 5, and 8 over at least two months at room temperature or an elevated temperature (e.g. at least three months, at least four months, at least five months, at least six months, at least twelve months). In certain embodiments, Impurity AA may be present in about 0.01 wt % to about 0.03 wt % of any of the products prepared from Examples 2, 4, 5, and 8 over at least six months at 40° C. with 75% relative humidity. In certain embodiments, the amount of Impurity AA is less than about 1.0 wt % of (R)-4-Amino-N-[(1-ethyl-2-pyrrolidinyl)methyl]-5-(ethylsulfonyl)-2-methoxybenzamide of Form A prepared from Examples 5 and 8. In certain embodiments, the amount of Impurity AA is less than about 1.0 wt % of (S)-4-Amino-N-[(1-ethyl-2-pyrrolidinyl)methyl]-5-(ethylsulfonyl)-2-methoxybenzamide of Form A′ prepared from Example 4. In certain embodiments, the amount of Impurity AA is less than about 0.2 wt % of (R)-4-Amino-N-[(1-ethyl-2-pyrrolidinyl)methyl]-5-(ethylsulfonyl)-2-methoxybenzamide of Form A prepared from Examples 5 and 8. In certain embodiments, the amount of Impurity AA is less than about 0.2 wt % of (S)-4-Amino-N-[(1-ethyl-2-pyrrolidinyl)methyl]-5-(ethylsulfonyl)-2-methoxybenzamide of Form A′ prepared from Example 4. In certain embodiments, Impurity AA may be present in about 0.001 wt % to about 0.3 wt % of any of the products prepared from Examples 1-8 or 10-15. In certain embodiments, Impurity AA may be present in about 0.01 wt % to about 0.2 wt % of any of the products prepared from Examples 1-8 or 10-15. In certain embodiments, Impurity AA may be present in about 0.001 wt % to about 0.3 wt % (e.g. 0.01 wt % to about 0.2 wt %) of any of the products prepared from Examples 1-8 or 10-15 over at least two months at room temperature or an elevated temperature (e.g. at least three months, at least four months, at least five months, at least six months, at least twelve months). In certain embodiments, Impurity AA may be present in about 0.01 wt % to about 0.03 wt % of any of the products prepared from Examples 1-8 or 10-15 over at least six months at 40° C. with 75% relative humidity. In certain embodiments, the amount of Impurity AA is less than about 1.0 wt % of (R)-4-Amino-N-[(1-ethyl-2-pyrrolidinyl)methyl]-5-(ethylsulfonyl)-2-methoxybenzamide of Form A prepared from Examples 3, 5, 7, 8, 12, 13, or 15. In certain embodiments, the amount of Impurity AA is less than about 1.0 wt % of (S)-4-Amino-N-[(1-ethyl-2-pyrrolidinyl)methyl]-5-(ethylsulfonyl)-2-methoxybenzamide of Form A′ prepared from Examples 1, 2, 4, 6, 10, 11, or 14. In certain embodiments, the amount of Impurity AA is less than about 0.2 wt % of (R)-4-Amino-N-[(1-ethyl-2-pyrrolidinyl)methyl]-5-(ethylsulfonyl)-2-methoxybenzamide of Form A prepared from Examples 3, 5, 7, 8, 12, 13, or 15. In certain embodiments, the amount of Impurity AA is less than about 0.2 wt % of (S)-4-Amino-N-[(1-ethyl-2-pyrrolidinyl)methyl]-5-(ethylsulfonyl)-2-methoxybenzamide of Form A′ prepared from Examples 1, 2, 4, 6, 10, 11, or 14.

In addition, Impurity BB-(i) of the following formula may be present in any of the products prepared from Example 5 or 8:

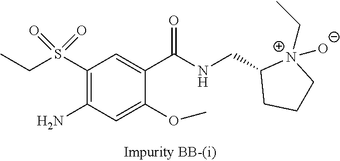 [SMILES: CC[N+]1([O-])CCC[C@@H]1CNC(=O)c1cc(S(=O)(=O)CC)c(N)cc1OC]

Impurity BB-(i) may also be present in any of the products prepared from Examples 13, 5, 7, 8, 12, 13, or 15. ¹H NMR (400 MHz, DMSO-d6) δ ppm 1.04-1.09 (m, 3H) 1.26 (t, J=7.04 Hz, 3H) 1.74 (br d, J=8.61 Hz, 1H) 1.82-1.92 (m, 1H) 1.94-2.07 (m, 2H) 3.05-3.17 (m, 4H) 3.20-3.31 (m, 2H) 3.40 (dt, J=14.87, 2.74 Hz, 1H) 3.49-3.58 (m, 1H) 3.77-3.81 (m, 3H) 3.81-3.88 (m, 1H) 6.45 (s, 1H) 6.52 (s, 2H) 8.07 (s, 1H) 9.67 (br d, J=4.70 Hz, 1H). ¹³C NMR (100 MHz, CHLOROFORM-d) δ ppm 7.24, 9.44, 20.25, 25.39, 37.72, 49.56, 56.17, 61.87, 66.96, 73.54, 98.38, 111.80, 112.18, 136.13, 151.08, 162.65, 164.52. In certain embodiments, the amount of Impurity BB-(i) is less than about 1.0 wt % of (R)-4-Amino-N-[(1-ethyl-2-pyrrolidinyl)methyl]-5-(ethylsulfonyl)-2-methoxybenzamide of Form A prepared from Example 5 or 8. In certain embodiments, the amount of Impurity BB is less than about 0.2 wt % of (R)-4-Amino-N-[(1-ethyl-2-pyrrolidinyl)methyl]-5-(ethylsulfonyl)-2-methoxybenzamide of Form A prepared from Example 5 or 8. In certain embodiments, the amount of Impurity BB-(i) is less than about 1.0 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity BB is less than about 0.2 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15.

In addition, Impurity BB-(ii) of the following formula may be present in any of the products prepared from Example 2 or 4:

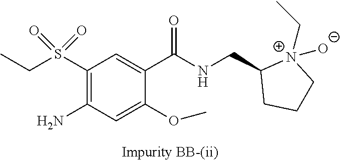 [SMILES: CC[N+]1([O-])CCC[C@H]1CNC(=O)c1cc(S(=O)(=O)CC)c(N)cc1OC]

It may also be present in any of the products from Examples 1, 2, 4, 6, 10, 11, or 14. ¹H NMR (400 MHz, CHLOROFORM-d) δ ppm 1.22 (t, J=7.24 Hz, 3H) 1.41 (t, J=7.04 Hz, 3H) 1.86 (br dd, J=10.17, 2.35 Hz, 1H) 2.04 (br dd, J=8.80, 4.50 Hz, 1H) 2.15-2.24 (m, 1H) 2.29 (br s, 1H) 3.04-3.29 (m, 4H) 3.38-3.51 (m, 2H) 3.56-3.66 (m, 2H) 3.87 (s, 3H) 4.06-4.14 (m, 1H) 5.83 (s, 2H) 6.23 (s, 1H) 8.41 (s, 1H) 8.90-8.97 (m, 1H). ¹³C NMR (100 MHz, CHLOROFORM-d) δ ppm 7.22, 9.42, 20.28, 25.34, 37.69, 49.57, 56.14, 61.93, 67.04, 73.49, 98.34, 111.89, 112.34, 136.14, 150.95, 162.61, 164.45. In certain embodiments, the amount of Impurity BB-(ii) is less than about 1.0 wt % of (S)-4-Amino-N-[(1-ethyl-2-pyrrolidinyl)methyl]-5-(ethylsulfonyl)-2-methoxybenzamide of Form A′ prepared from Example 2 or 4. In certain embodiments, the amount of Impurity BB-(ii) is less than about 0.2 wt % of (S)-4-Amino-N-[(1-ethyl-2-pyrrolidinyl)methyl]-5-(ethylsulfonyl)-2-methoxybenzamide of Form A′ prepared from Example 2 or 4. In certain embodiments, the amount of Impurity BB-(ii) is less than about 1.0 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14. In certain embodiments, the amount of Impurity BB-(ii) is less than about 0.2 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14.

In addition, Impurity CC-(i) of the following formula may be present in any of the products prepared from Examples 3, 5, 7, 8, 12, 13, or 15:

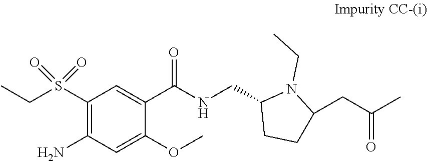 [SMILES: CCN1C(CC(C)=O)CC[C@@H]1CNC(=O)c1cc(S(=O)(=O)CC)c(N)cc1OC]

In certain embodiments, the amount of Impurity CC-(i) is less than about 1.0 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity CC-(i) is less than about 0.2 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity CC-(i) is less than about 0.15 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity CC-(i) is less than about 0.05 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15.

In addition, Impurity CC-(ii) of the following formula may be present in any of the products prepared from Example 1, 2, 4, 6, 10, 11, or 14.

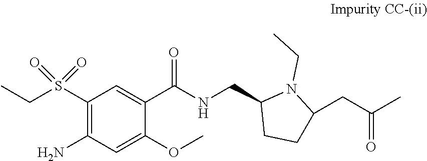 [SMILES: CCN1C(CC(C)=O)CC[C@H]1CNC(=O)c1cc(S(=O)(=O)CC)c(N)cc1OC]

In certain embodiments, the amount of Impurity CC-(ii) is less than about 1.0 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14. In certain embodiments, the amount of Impurity CC-(ii) is less than about 0.2 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14. In certain embodiments, the amount of Impurity CC-(ii) is less than about 0.15 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14. In certain embodiments, the amount of Impurity CC-(ii) is less than about 0.05 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14.

In addition, Impurity DD-(i) of the following formula may be present in any of the products prepared from Example 3, 5, 7, 8, 12, 13, or 15:

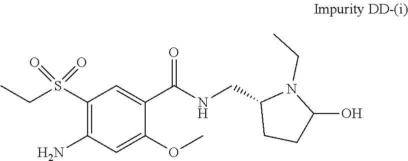 [SMILES: CCN1C(O)CC[C@@H]1CNC(=O)c1cc(S(=O)(=O)CC)c(N)cc1OC]

In certain embodiments, the amount of Impurity DD-(i) is less than about 1.0 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity DD-(i) is less than about 0.2 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity DD-(i) is less than about 0.15 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity DD-(i) is less than about 0.05 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15.

In addition, Impurity DD-(ii) of the following formula may be present in any of the products prepared from Example 1, 2, 4, 6, 10, 11, and 14:

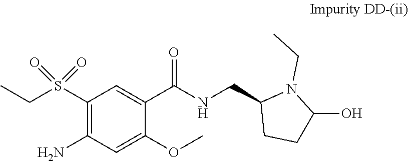 [SMILES: CCN1C(O)CC[C@H]1CNC(=O)c1cc(S(=O)(=O)CC)c(N)cc1OC]

In certain embodiments, the amount of Impurity DD-(ii) is less than about 1.0 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14. In certain embodiments, the amount of Impurity DD-(ii) is less than about 0.2 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14. In certain embodiments, the amount of Impurity DD-(ii) is less than about 0.15 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14. In certain embodiments, the amount of Impurity DD-(ii) is less than about 0.05 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14.

In addition, Impurity EE of the following formula may be present in any of the products prepared from the examples:

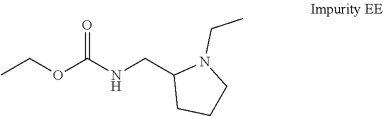

In certain embodiments, the amount of Impurity EE is less than about 1.0 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity EE is less than about 0.2 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity EE is less than about 0.15 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity EE is less than about 0.05 wt % of (R)-4-Amino-N-[(1-ethyl-2-pyrrolidinyl)methyl]-5-(ethylsulfonyl)-2-methoxybenzamide of Form A prepared from Example 3, 5, 7, 8, 12, 13, or 15. In certain embodiments, the amount of Impurity EE is less than about 1.0 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14. In certain embodiments, the amount of Impurity EE is less than about 0.2 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14. In certain embodiments, the amount of Impurity EE is less than about 0.15 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14. In certain embodiments, the amount of Impurity EE is less than about 0.05 wt % of (S)-4-Amino-N-[(1-ethyl-2-pyrrolidinyl)methyl]-5-(ethylsulfonyl)-2-methoxybenzamide of Form A′ prepared from Example 1, 2, 4, 6, 10, 11, or 14.

In addition, isopropyl acetate may be observed in any of the products of the examples. In certain embodiments, isopropyl acetate may be present in any of the products prepared from the examples in an amount that is less than about 5,000 ppm. In certain embodiments, isopropyl acetate may be present in any of the products prepared from the examples in an amount that is less than about 1,000 ppm. In certain embodiments, isopropyl acetate may be present in any of the products prepared from the examples in an amount that is less than about 500 ppm. In certain embodiments, isopropyl acetate may be present in any of the products prepared from the examples in an amount that is from about 200 ppm to about 300 ppm. In certain embodiments, isopropyl acetate may be present in any of the products prepared from the examples in an amount that is less than about 100 ppm. In certain embodiments, isopropyl acetate may be present in any of the products prepared from the examples in an amount that is less than about 50 ppm.

In addition, methyl tert-butyl ether may be observed in any of the products prepared from the examples. In certain embodiments, methyl tert-butyl ether may be present in any of the products prepared from the examples in an amount that is less than about 5,000 ppm. In certain embodiments, methyl tert-butyl ether may be present in any of the products prepared from the examples in an amount that is less than about 1,000 ppm. In certain embodiments, methyl tert-butyl ether may be present in any of the products prepared from the examples in an amount that is less than about 500 ppm. In certain embodiments, methyl tert-butyl ether may be present in any of the products prepared from the examples in an amount that is less than about 200 ppm. In certain embodiments, methyl tert-butyl ether may be present in any of the products prepared from the examples in an amount that is from about 75 ppm to about 125 ppm. In certain embodiments, methyl tert-butyl ether may be present in any of the products prepared from the examples in an amount that is less than about 100 ppm. In certain embodiments, methyl tert-butyl ether may be present in any of the products prepared from the examples in an amount that is less than about 50 ppm. In certain embodiments, methyl tert-butyl ether may be present in any of the products prepared from the examples in an amount that is less than about 10 ppm.

### Additional Embodiments

Embodiment 1. A method of preparing an enantiomerically pure crystalline form of (R)-(+)-amisulpride comprising the steps of:
(a) coupling, in the presence of a tertiary amine and an acid activating reagent, 4-amino-5-(ethylsulfonyl)-2-methoxybenzoic acid and (R)-(1-ethylpyrrolidin-2-yl) methanamine to form a reaction mixture; and
(b) isolating from the reaction mixture of step (a), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°;
(c) wherein step (b) does not comprise formation of a solvate of (R)-(+)-amisulpride.

Embodiment 2. The method of embodiment 1, wherein the coupling of step (a) comprises the steps of:
(a1) reacting 4-amino-5-(ethylsulfonyl)-2-methoxybenzoic acid with the tertiary amine and the acid activating reagent to form a first reaction mixture; and
(a2) adding (R)-(1-ethylpyrrolidin-2-yl)methanamine to the first reaction mixture.

Embodiment 3. A method of preparing an enantiomerically pure crystalline form of (S)-(−)-amisulpride comprising the steps of:
(a) coupling, in the presence of a tertiary amine and an acid activating reagent, 4-amino-5-(ethylsulfonyl)-2-methoxybenzoic acid and (S)-(1-ethylpyrrolidin-2-yl) methanamine to form a reaction mixture; and
(b) isolating from the reaction mixture of step (a), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°;
(c) wherein the step (b) does not comprise formation of a solvate of (S)-(−)-amisulpride.

Embodiment 4. The method of embodiment 3, wherein the coupling of step (a) comprises the steps of:
(a1) reacting 4-amino-5-(ethylsulfonyl)-2-methoxybenzoic acid with the tertiary amine and the acid activating reagent to form a first reaction mixture; and
(a2) adding (S)-(1-ethylpyrrolidin-2-yl)methanamine to the first reaction mixture.

Embodiment 5. The method of any one of embodiments 1-4, wherein step (b) comprises step (b1): concentrating the reaction mixture of step (a) to give a mixture of step (b1).

Embodiment 6. The method of embodiment 5, further comprising step (b2): treating the mixture of step (b1) with a base and the isolation reagent to form a step (b2) mixture.

Embodiment 7. The method of embodiment 6, wherein the base is an aqueous solution of an inorganic base.

Embodiment 8. The method of embodiment 7, wherein the base is an aqueous solution of potassium carbonate.

Embodiment 9. The method of any one of embodiments 6-8, further comprising step (b3): separating the step (b2) mixture to obtain an organic phase.

Embodiment 10. The method of embodiment 9, further comprising step (b4): concentrating the organic phase to a first concentrated solution having less than about 2 wt % water.

Embodiment 11. The method of embodiment 10, wherein the first concentrated solution has about 1.0 wt % to about 0.001 wt % water.

Embodiment 12. The method of embodiment 11, wherein the first concentrated solution has about 1.0 wt % to about 0.01 wt % water.

Embodiment 13. The method of embodiment 12, wherein the first concentrated solution has about 0.5 wt % to about 0.01 wt % water.

Embodiment 14. The method of any one of embodiments 10-13, further comprising step (b5): concentrating the first concentrated solution to a second concentrated solution, wherein the second concentrated solution has a total weight of (R)-(+)-amisulpride or (S)-(−)-amisulpride that is about 15 wt % to about 65 wt % of the second concentrated solution.

Embodiment 15. The method of embodiment 14, wherein the second concentrated solution has a total weight of (R)-(+)-amisulpride or (S)-(−)-amisulpride that is about 35 wt % to about 40 wt % of the second concentrated solution.

Embodiment 16. The method of embodiment 14, wherein the second concentrated solution has a total weight of (R)-(+)-amisulpride or (S)-(−)-amisulpride that is about 33 wt % to about 38 wt % of the second concentrated solution.

Embodiment 17. The method of embodiment 14, wherein the second concentrated solution has a total weight of (R)-(+)-amisulpride or (S)-(−)-amisulpride that is about 35 wt % of the second concentrated solution.

Embodiment 18. The method of any one of embodiments 14-17, further comprising adding S5, wherein S5 is an ether solvent, to the second concentrated solution.

Embodiment 19. The method of embodiment 18, wherein S5 is methyl tert-butyl ether.

Embodiment 20. The method of any one of embodiments 14-19, further comprising step (b6): adding a seed amount of a crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2° to the second concentrated solution to form a seeded mixture.

Embodiment 21. The method of any one of embodiments 14-19, further comprising step (b6): adding a seed amount of a crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2° to the second concentrated solution to form a seeded mixture.

Embodiment 22. The method of embodiment 20 or 21, wherein the seed amount has a total weight that is about 0.001 wt % to about 15 wt % of the second concentrated solution.

Embodiment 23. The method of embodiment 20 or 21, wherein the seed amount has a total weight that is about 0.1 wt % to about 10 wt % of the second concentrated solution.

Embodiment 24. The method of embodiment 20 or 21, wherein the seed amount has a total weight that is about 0.1 wt % to about 5 wt % of the expected yield of the (R)-(+)-amisulpride or (S)-(−)-amisulpride present in the second concentrated solution.

Embodiment 25. The method of embodiment 20 or 21, wherein the seed amount has a total weight that is about 0.1 wt % to about 2.0 wt % of the expected yield of the (R)-(+)-amisulpride or (S)-(−)-amisulpride present in the second concentrated solution.

Embodiment 26. The method of embodiment 20 or 21, wherein the seed amount has a total weight that is about 0.4 wt %, about 1.4 wt %, or about 2.0 wt % of the expected yield of the (R)-(+)-amisulpride or (S)-(−)-amisulpride present in the second concentrated solution.

Embodiment 27. The method of embodiment 20 or 21, wherein the seed amount has a total weight that is about 0.75 wt % of the expected yield of the (R)-(+)-amisulpride or (S)-(−)-amisulpride present in the second concentrated solution.

Embodiment 28. The method of any one of embodiments 20-27, further comprising step (b7): filtering the seeded mixture of step (b6) to obtain a product solid.

Embodiment 29. The method of embodiment 28, further comprising step (b8): drying the product solid to obtain a crude product.

Embodiment 30. The method of embodiment 29, further comprising step (b9): recrystallizing the crude product in the presence of the isolation reagent.

Embodiment 31. The method of embodiment 30, wherein the recrystallizing comprises (i) dissolving the crude product with the isolation reagent to form a recrystallization solution, (ii) filtering and concentrating the recrystallization solution, and (iii) adding a seed amount of a crystalline form of (R)-(+)-amisulpride or a crystalline form of (S)-(−)-amisulpride.

Embodiment 32. The method of embodiment 30, wherein the recrystallizing comprises (i) heating the crude product in the presence of an isolation reagent at a first elevated temperature to form a recrystallization solution, (ii) filtering and concentrating the recrystallization solution to form a concentrated recrystallization solution; (iii) adding S5, wherein S5 is a solvent, to the concentrated recrystallization solution; (iv) adding a seed amount of a crystalline form of (R)-(+)-amisulpride or a crystalline form of (S)-(−)-amisulpride to the concentrated recrystallization solution of step (iii) to form a seeded recrystallization solution; and (v) cooling the seeded recrystallization solution.

Embodiment 33. The method of embodiment 32, wherein the first elevated temperature is from about 50° C. to about 55° C.

Embodiment 34. The method of embodiment 32 or 33, wherein the cooling comprises cooling to a first reduced temperature over a first period of time, and then cooling to a second reduced temperature over a second period of time.

Embodiment 35. The method of embodiment 34, wherein the first reduced temperature is from about 38° C. to about 42° C.

Embodiment 36. The method of embodiment 34 or 35, wherein the first period of time is about 120 min.

Embodiment 37. The method of any one of embodiments 34-36, wherein the second reduced temperature is from about 10° C. to about 15° C.

Embodiment 38. The method of any one of embodiments 34-37, wherein the second period of time is about 60 min.

Embodiment 39. The method of any one of embodiments 34-38, wherein the cooling over the first period of time is performed at a first cooling rate.

Embodiment 40. The method of any one of embodiments 34-39, wherein the cooling over the second period of time is performed at a second cooling rate.

Embodiment 41. The method of embodiment 40, wherein the second cooling rate is greater than the first cooling rate.

Embodiment 42. The method of embodiment 39, wherein the first cooling rate is about 0.1° C./min.

Embodiment 43. The method of embodiment 40, wherein the second cooling rate is about 0.5° C./min.

Embodiment 44. A method of preparing an enantiomerically pure crystalline form of (R)-(+)-amisulpride comprising the steps of:
(a) reacting 4-amino-5-(ethylsulfonyl)-2-methoxybenzoic acid with a tertiary amine and an acid activating reagent to form a reaction mixture;
(b) adding a (R)-(1-ethylpyrrolidin-2-yl)methanamine salt to the reaction mixture to form a mixture of step (b); and
(c) isolating from the mixture of step (b), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2°.

Embodiment 45. The method of embodiment 44, wherein step (c) does not comprise formation of a solvate of (R)-(+)-amisulpride.

Embodiment 46. The method of embodiment 44 or 45, wherein the isolating of step (c) comprises
(d1) adding the isolation reagent to the mixture of step (b), followed by an aqueous base solution to form a mixture of step (d1),
(d2) separating the mixture of step (d1) to obtain an organic phase;
(d3) concentrating the organic phase of step (d2) to produce a product solution having about 5.0 wt % to about 0.001 wt % water;
(d4) adding to the product solution a seed amount of a crystalline form of (R)-(+)-amisulpride, characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (R)-(+)-amisulpride.

Embodiment 47. The method of any one of embodiments 44-46, further comprising recrystallizing the enantiomerically pure crystalline form of (R)-(+)-amisulpride of step (d6).

Embodiment 48. The method of embodiment 47, wherein the recrystallizing comprises (i) heating the enantiomerically pure crystalline form of (R)-(+)-amisulpride of step (d6) in the presence of an isolation reagent at a first elevated temperature to form a recrystallization solution, (ii) filtering and concentrating the recrystallization solution to form a concentrated recrystallization solution; (iii) adding S5, wherein S5 is a solvent, to the concentrated recrystallization solution; (iv) adding a seed amount of a crystalline form of (R)-(+)-amisulpride to the concentrated recrystallization solution of step (iii) to form a seeded recrystallization solution; and (v) cooling the seeded recrystallization solution.

Embodiment 49. The method of embodiment 48, wherein the first elevated temperature is from about 50° C. to about 55° C.

Embodiment 50. The method of embodiment 48 or 49, wherein the cooling comprises cooling to a first reduced temperature over a first period of time, and then cooling to a second reduced temperature over a second period of time.

Embodiment 51. The method of embodiment 50, wherein the first reduced temperature is from about 38° C. to about 42° C.

Embodiment 52. The method of embodiment 50 or 51, wherein the first period of time is about 120 min.

Embodiment 53. The method of any one of embodiments 50-52, wherein the second reduced temperature is from about 10° C. to about 15° C.

Embodiment 54. The method of any one of embodiments 50-53, wherein the second period of time is about 60 min.

Embodiment 55. The method of any one of embodiments 48-54, wherein S5 is an ether solvent.

Embodiment 56. The method of any one of embodiments 48-54, wherein S5 is methyl tert-butyl ether.

Embodiment 57. The method of any one of embodiments 48-56, wherein the method further comprises isolating and drying the crystalline form of (R)-(+)-amisulpride.

Embodiment 58. A method of preparing an enantiomerically pure crystalline form of (S)-(−)-amisulpride comprising the steps of:
(a) reacting 4-amino-5-(ethylsulfonyl)-2-methoxybenzoic acid with a tertiary amine and an acid activating reagent to form a reaction mixture;
(b) adding a (S)-(1-ethylpyrrolidin-2-yl)methanamine salt to the reaction mixture of step (a) to form a mixture of step (b); and
(c) isolating from the mixture of step (b), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ), at least at 7.0±0.2° and 9.7±0.2°, and further peaks at 15.4±0.2° and/or 19.4±0.2°.

Embodiment 59. The method of embodiment 58, wherein step (c) does not comprise formation of a solvate of (S)-(−)-amisulpride.

Embodiment 60. The method of embodiment 58 or 59, wherein the isolating of step (c) comprises:
(d1) adding the isolation reagent to the mixture of step (b), followed by an aqueous base solution to form a mixture of step (d1);
(d2) separating the mixture of step (d1) to obtain an organic phase;
(d3) concentrating the organic phase of step (d2) to produce a product solution with about 5.0 wt % to about 0.01 wt % water;
(d4) adding to the product solution of step (d3) with a seed amount of a crystalline form of (S)-(−)-amisulpride characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (S)-(−)-amisulpride.

Embodiment 61. The method of any one of embodiments 58-60, further comprising recrystallizing the enantiomerically pure crystalline form of (S)-(−)-amisulpride of step (d6).

Embodiment 62. The method of embodiment 61, wherein the recrystallizing comprises (i) heating the enantiomerically pure crystalline form of (S)-(−)-amisulpride of step (d6) in the presence of an isolation reagent at a first elevated temperature to form a recrystallization solution, (ii) filtering and concentrating the recrystallization solution to form a concentrated recrystallization solution; (iii) adding S5, wherein S5 is a solvent, to the concentrated recrystallization solution; (iv) adding a seed amount of a crystalline form of (S)-(−)-amisulpride to the concentrated recrystallization solution of step (iii) to form a seeded recrystallization solution; and (v) cooling the seeded recrystallization solution.

Embodiment 63. The method of embodiment 61 or 62, wherein the first elevated temperature is from about 50° C. to about 55° C.

Embodiment 64. The method of embodiment 61 or 62, wherein the cooling comprises cooling to a first reduced temperature over a first period of time, and then cooling to a second reduced temperature over a second period of time.

Embodiment 65. The method of embodiment 64, wherein the first reduced temperature is from about 38° C. to about 42° C.

Embodiment 66. The method of embodiment 64 or 65, wherein the first period of time is about 120 min.

Embodiment 67. The method of any one of embodiments 64-66, wherein the second reduced temperature is from about 10° C. to about 15° C.

Embodiment 68. The method of any one of embodiments 64-67, wherein the second period of time is about 60 min.

Embodiment 69. The method of any one of embodiments 62-68, wherein S5 is an ether solvent.

Embodiment 70. The method of any one of embodiments 62-68, wherein S5 is methyl tert-butyl ether.

Embodiment 71. The method of embodiment 46 or 60, wherein the product solution of step (d3) has about 0.001 wt % to about 0.5 wt % water.

Embodiment 72. The method of any one of embodiments 1-71, wherein the tertiary amine is of the formula

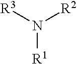

wherein:
   (i) R¹, R² and R³ are each independently C₁₋₆ alkyl, C₃₋₆ cycloalkyl, 3-10 membered monocyclic or bicyclic heterocycloalkyl, or 5-10 membered monocyclic heteroaryl; or
   (ii) R¹ is C₁₋₆ alkyl, C₃₋₆ cycloalkyl, 3-10 membered monocyclic or bicyclic heterocycloalkyl, or 5-10 membered monocyclic heteroaryl, and R² and R³ together with the N atom to which they are attached form a 3-10 membered monocyclic or bicyclic heterocycloalkyl or a 5-10 membered monocyclic heteroaryl.

Embodiment 73. The method of embodiment 72, wherein R² and R³ together with the N atom to which they are attached form a 3-10 membered monocyclic or bicyclic heterocycloalkyl or a 5-10 membered monocyclic heteroaryl.

Embodiment 74. The method of any one of embodiments 1-73, wherein the tertiary amine is triethyl amine.

Embodiment 75. The method of any one of embodiments 1-73, wherein the tertiary amine is 4-methylmorpholine.

Embodiment 76. The method of any one of embodiments 1-75, wherein the acid activating reagent is of the formula

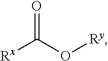

wherein R^{x} is halogen and R^{y} is C₁₋₅ alkyl.

Embodiment 77. The method of any one of embodiments 1-75, wherein the acid activating reagent is ethyl chloroformate.

Embodiment 78. The method of any one of embodiments 1-77, wherein the isolation reagent is of the formula

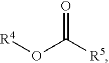

wherein R⁴ is C₁₋₆ alkyl; and R⁵ is C₁₋₅ alkyl or C₁₋₅ alkoxide.

Embodiment 79. A method of preparing an enantiomerically pure crystalline form of (R)-(+)-amisulpride comprising the steps of:
(a) heating (R)-(+)-amisulpride in the presence of an isolation reagent to a first elevated temperature to form a crystallization mixture;
(b) adding a seed amount of Form A of (R)-(+)-amisulpride to the crystallization mixture to form a seeded mixture;
(c) cooling the seeded mixture to a first reduced temperature over a first period of time;
(d) cooling the seeded mixture from step (c) to a second reduced temperature over a second period of time; and
(e) filtering the seeded mixture from step (d) to provide an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°.

Embodiment 80. A method of preparing an enantiomerically pure crystalline form of (S)-(−)-amisulpride comprising the steps of:
(a) heating (S)-(−)-amisulpride in the presence of an isolation reagent to a first elevated temperature to form a crystallization mixture;
(b) adding a seed amount of Form A′ of (S)-(−)-amisulpride to the crystallization mixture to form a seeded mixture;
(c) cooling the seeded mixture to a first reduced temperature over a first period of time;
(d) cooling the seeded mixture from step (c) to a second reduced temperature over a second period of time; and
(e) filtering the seeded mixture from step (d) to provide an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°.

Embodiment 81. The method of embodiment 79 or 80, wherein the first elevated temperature is from about 50° C. to about 55° C.

Embodiment 82. The method of any one of embodiments 79-81, wherein the first reduced temperature is from about 38° C. to about 42° C.

Embodiment 83. The method of any one of embodiments 79-82, wherein the first period of time is about 120 min.

Embodiment 84. The method of any one of embodiments 79-83, wherein the second reduced temperature is from about 10° C. to about 15° C.

Embodiment 85. The method of any one of embodiments 79-84, wherein the second period of time is about 60 min.

Embodiment 86. The method of any one of embodiments 79-85, wherein the heating of step (a) is further performed in the presence of S5, wherein S5 is an ether solvent.

Embodiment 87. The method of embodiment 86, wherein S5 is methyl tert-butyl ether.

Embodiment 88. The method of any one of embodiments 1-87, wherein the isolation reagent is of the formula

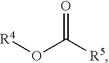

wherein R⁴ is C₃₋₆ alkyl; and R⁵ is C₁₋₅ alkyl.

Embodiment 89. The method of any one of embodiments 1-88, wherein the isolation reagent is not ethyl acetate.

Embodiment 90. The method of any one of embodiments 1-88, wherein the isolation reagent is isopropyl acetate.

Embodiment 91. The method of any one of embodiments 1-87, wherein the isolation reagent is of the formula

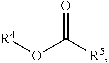

wherein R⁴ is C₁₋₆ alkyl; and R⁵ is C₁₋₅ alkoxide.

Embodiment 92. The method of any one of embodiments 1-87, wherein the isolation reagent is diethyl carbonate or dimethyl carbonate.

Embodiment 93. The method of any one of embodiments 1-87, wherein the isolation reagent is of the formula R⁶COR⁷, where each of R⁶ and R⁷ is independently C₁₋₅ alkyl.

Embodiment 94. The method of any one of embodiments 44-57, 71-78, and 88-93, wherein the (R)-(1-ethylpyrrolidin-2-yl)methanamine salt is a bis tartrate salt of (R)-(1-ethylpyrrolidin-2-yl)methanamine.

Embodiment 95. The method of any one of embodiments 44-57, 71-78, and 88-93, wherein the (R)-(1-ethylpyrrolidin-2-yl)methanamine salt is a bis L-tartrate salt of (R)-(1-ethylpyrrolidin-2-yl)methanamine.

Embodiment 96. The method of any one of embodiments 58-78 and 88-93, wherein the (S)-(1-ethylpyrrolidin-2-yl)methanamine salt is a bis tartrate salt of (S)-(1-ethylpyrrolidin-2-yl)methanamine.

Embodiment 97. The method of any one embodiments 58-78 and 88-93, wherein the (S)-(1-ethylpyrrolidin-2-yl)methanamine salt is a bis D-tartrate salt of (S)-(1-ethylpyrrolidin-2-yl)methanamine.

Embodiment 98. The method of any one of embodiments 1-87, further comprising recrystallizing the enantiomerically pure crystalline form of (R)-(+)-amisulpride or the enantiomerically pure crystalline form of (S)-(−)-amisulpride in the presence of isopropyl acetate.

Embodiment 99. The method of any one of embodiments 20, 22-43, 46-57, 71-78, 79, 81-95, and 98, wherein the seed amount of a crystalline form of (R)-(+)-amisulpride has a greater than about 95% chemical purity and a greater than about 95% enantiomeric purity.

Embodiment 100. The method of any one of embodiments 20, 22-43, 46-57, 71-78, 79, 81-95, and 98, wherein the seed amount of a crystalline form of (R)-(+)-amisulpride has a greater than about 98% chemical purity and a greater than about 98% enantiomeric purity.

Embodiment 101. The method of any one of embodiments 21-43, 60-78, 80-93, and 96-98, wherein the seed amount of a crystalline form of (S)-(−)-amisulpride has a greater than about 95% chemical purity and a greater than about 95% enantiomeric purity.

Embodiment 102. The method of any one of embodiments 21-43, 60-78, 80-93, and 96-98, wherein the seed amount of a crystalline form of (S)-(−)-amisulpride has a greater than about 98% chemical purity and a greater than about 98% enantiomeric purity.

Embodiment 103. The method of any one of embodiments 1, 2, 5-57, 71-79, 81-95, and 98-100, wherein the enantiomerically pure crystalline form of (R)-(+)-amisulpride has a greater than about 95% chemical purity.

Embodiment 104. The method of any one of embodiments 1, 2, 5-57, 71-79, 81-95, and 98-100, wherein the enantiomerically pure crystalline form of (R)-(+)-amisulpride has a greater than about 98% chemical purity.

Embodiment 105. The method of any one of embodiments 1, 2, 5-57, 71-79, 81-95, and 98-100, wherein the enantiomerically pure crystalline form of (R)-(+)-amisulpride has a greater than about 99% chemical purity.

Embodiment 106. The method of any one of embodiments 1, 2, 5-57, 71-79, 81-95, and 98-100, wherein the enantiomerically pure crystalline form of (R)-(+)-amisulpride has a greater than about 95% enantiomeric purity.

Embodiment 107. The method of any one of embodiments 1, 2, 5-57, 71-79, 81-95, and 98-100, wherein the enantiomerically pure crystalline form of (R)-(+)-amisulpride has a greater than about 98% enantiomeric purity.

Embodiment 108. The method of any one of embodiments 1, 2, 5-57, 71-79, 81-95, and 98-100, wherein the enantiomerically pure crystalline form of (R)-(+)-amisulpride has a greater than about 99% enantiomeric purity.

Embodiment 109. The method of any one of embodiments 3-43, 58-78, 80-93, 96-98, 101, and 102, wherein the enantiomerically pure crystalline form of (S)-(−)-amisulpride has a greater than about 95% chemical purity.

Embodiment 110. The method of any one of embodiments 3-43, 58-78, 80-93, 96-98, 101, and 102, wherein the enantiomerically pure crystalline form of (S)-(−)-amisulpride has a greater than about 98% chemical purity.

Embodiment 111. The method of any one of embodiments 3-43, 58-78, 80-93, 96-98, 101, and 102, wherein the enantiomerically pure crystalline form of (S)-(−)-amisulpride has a greater than about 99% chemical purity.

Embodiment 112. The method of any one of embodiments 3-43, 58-78, 80-93, 96-98, 101, and 102, wherein the enantiomerically pure crystalline form of (S)-(−)-amisulpride has a greater than about 95% enantiomeric purity.

Embodiment 113. The method of any one of embodiments 3-43, 58-78, 80-93, 96-98, 101, and 102, wherein the enantiomerically pure crystalline form of (S)-(−)-amisulpride has a greater than about 98% enantiomeric purity.

Embodiment 114. The method of any one of embodiments 3-43, 58-78, 80-93, 96-98, 101, and 102, wherein the enantiomerically pure crystalline form of (S)-(−)-amisulpride has a greater than about 99% enantiomeric purity.

Embodiment 115. An enantiomerically pure crystalline form of (R)-(+)-amisulpride, wherein the crystalline form of (R)-(+)-amisulpride has a particle size distribution D₁₀ value of about 2 to about 10 μm; a particle size distribution D₅₀ value of about 15 to about 30 μm; and a particle size distribution D₉₀ value of about 75 to about 100 μm, as measured by a laser diffraction particle size analyzer.

Embodiment 116. An enantiomerically pure crystalline form of (S)-(−)-amisulpride, wherein the crystalline form of (S)-(−)-amisulpride has a particle size distribution D₁₀ value of about 2 to about 10 μm; a particle size distribution D₅₀ value of about 15 to about 30 μm; and a particle size distribution D₉₀ value of about 75 to about 100 μm, as measured by a laser diffraction particle size analyzer.

Embodiment 117. An enantiomerically pure crystalline form of (R)-(+)-amisulpride or (S)-(−)-amisulpride prepared by the method of any one of embodiments 1-114, comprising less than about 1.0 wt % of a compound of the following formula:

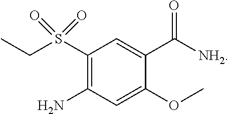 [SMILES: CCS(=O)(=O)c1cc(C(N)=O)c(OC)cc1N]

Embodiment 118. An enantiomerically pure crystalline form of (R)-(+)-amisulpride or (S)-(−)-amisulpride prepared by the method of any one of embodiments 1-114, comprising less than about 0.2 wt % of a compound of the following formula:

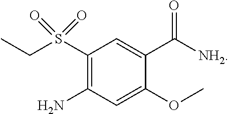 [SMILES: CCS(=O)(=O)c1cc(C(N)=O)c(OC)cc1N]

Embodiment 119. An enantiomerically pure crystalline form of (R)-(+)-amisulpride or (S)-(−)-amisulpride prepared by the method of any one of embodiments 1-114, comprising less than about 1.0 wt % of a compound of the following formula:

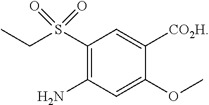 [SMILES: CCS(=O)(=O)c1cc(C(=O)O)c(OC)cc1N]

Embodiment 120. An enantiomerically pure crystalline form of (R)-(+)-amisulpride or (S)-(−)-amisulpride prepared by the method of any one of embodiments 1-114, comprising less than about 0.2 wt % of a compound of the following formula:

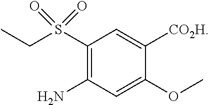 [SMILES: CCS(=O)(=O)c1cc(C(=O)O)c(OC)cc1N]

Embodiment 121. An enantiomerically pure crystalline form of (R)-(+)-amisulpride or (S)-(−)-amisulpride prepared by the method of any one of embodiments 1-114, comprising less than about 1.0 wt % of a compound of the following formula:

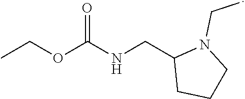

Embodiment 122. An enantiomerically pure crystalline form of (R)-(+)-amisulpride or (S)-(−)-amisulpride prepared by the method of any one of embodiments 1-114, comprising less than about 0.2 wt % of a compound of the following formula:

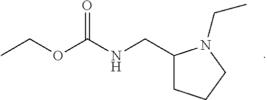

Embodiment 123. An enantiomerically pure crystalline form of (R)-(+)-amisulpride prepared by the method of any one of embodiments 1, 2, 5-57, 71-79, 81-95, 98-100, and 103-108, comprising less than about 1.0 wt % of a compound of the following formula:

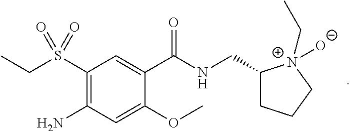 [SMILES: CC[N+]1([O-])CCC[C@@H]1CNC(=O)c1cc(S(=O)(=O)CC)c(N)cc1OC]

Embodiment 124. An enantiomerically pure crystalline form of (R)-(+)-amisulpride prepared by the method of any one of embodiments 1, 2, 5-57, 71-79, 81-95, 98-100, and 103-108, comprises less than about 0.2 wt % of a compound of the following formula:

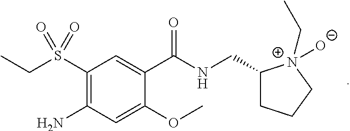 [SMILES: CC[N+]1([O-])CCC[C@@H]1CNC(=O)c1cc(S(=O)(=O)CC)c(N)cc1OC]

Embodiment 125. An enantiomerically pure crystalline form of (R)-(+)-amisulpride prepared by the method of any one of embodiments 1, 2, 5-57, 71-79, 81-95, 98-100, and 103-108, comprising less than about 1.0 wt % of a compound of the following formula:

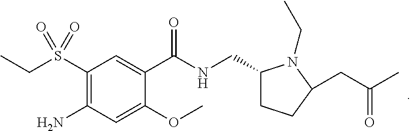 [SMILES: CCN1C(CC(C)=O)CC[C@@H]1CNC(=O)c1cc(S(=O)(=O)CC)c(N)cc1OC]

Embodiment 126. An enantiomerically pure crystalline form of (R)-(+)-amisulpride prepared by the method of any one of embodiments 1, 2, 5-57, 71-79, 81-95, 98-100, and 103-108, comprises less than about 0.2 wt % of a compound of the following formula:

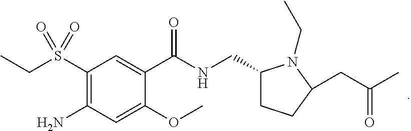 [SMILES: CCN1C(CC(C)=O)CC[C@@H]1CNC(=O)c1cc(S(=O)(=O)CC)c(N)cc1OC]

Embodiment 127. An enantiomerically pure crystalline form of (R)-(+)-amisulpride prepared by the method of any one of embodiments 1, 2, 5-57, 71-79, 81-95, 98-100, and 103-108, comprising less than about 1.0 wt % of a compound of the following formula:

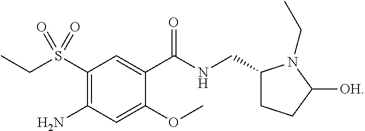 [SMILES: CCN1C(O)CC[C@@H]1CNC(=O)c1cc(S(=O)(=O)CC)c(N)cc1OC]

Embodiment 128. An enantiomerically pure crystalline form of (R)-(+)-amisulpride prepared by the method of any one of embodiments 1, 2, 5-57, 71-79, 81-95, 98-100, and 103-108, comprises less than about 0.2 wt % of a compound of the following formula:

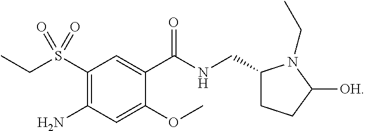 [SMILES: CCN1C(O)CC[C@@H]1CNC(=O)c1cc(S(=O)(=O)CC)c(N)cc1OC]

Embodiment 129. An enantiomerically pure crystalline form of (S)-(−)-amisulpride prepared by the method of any one of embodiments 3-43, 58-78, 80-93, 96-98, 101, 102, and 110-114, comprising less than about 1.0 wt % of a compound of the following formula:

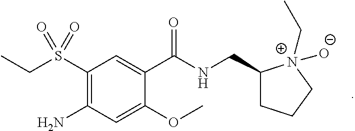 [SMILES: CC[N+]1([O-])CCC[C@H]1CNC(=O)c1cc(S(=O)(=O)CC)c(N)cc1OC]

Embodiment 130. An enantiomerically pure crystalline form of (S)-(−)-amisulpride prepared by the method of any one of embodiments 3-43, 58-78, 80-93, 96-98, 101, 102, and 110-114, comprising less than about 0.2 wt % of a compound of the following formula:

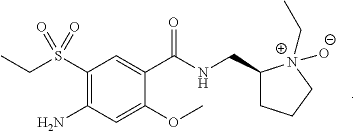 [SMILES: CC[N+]1([O-])CCC[C@H]1CNC(=O)c1cc(S(=O)(=O)CC)c(N)cc1OC]

Embodiment 131. An enantiomerically pure crystalline form of (S)-(−)-amisulpride prepared by the method of any one of embodiments 3-43, 58-78, 80-93, 96-98, 101, 102, and 110-114, comprising less than about 1.0 wt % of a compound of the following formula:

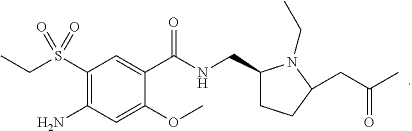 [SMILES: CCN1C(CC(C)=O)CC[C@H]1CNC(=O)c1cc(S(=O)(=O)CC)c(N)cc1OC]

Embodiment 132. An enantiomerically pure crystalline form of (S)-(−)-amisulpride prepared by the method of any one of embodiments 3-43, 58-78, 80-93, 96-98, 101, 102, and 110-114, comprising less than about 0.2 wt % of a compound of the following formula:

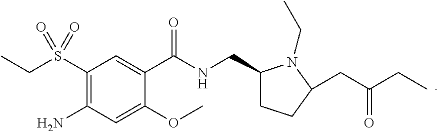 [SMILES: CCC(=O)CC1CC[C@@H](CNC(=O)c2cc(S(=O)(=O)CC)c(N)cc2OC)N1CC]

Embodiment 133. An enantiomerically pure crystalline form of (S)-(−)-amisulpride prepared by the method of any one of embodiments 3-43, 58-78, 80-93, 96-98, 101, 102, and 110-114, comprising less than about 1.0 wt % of a compound of the following formula:

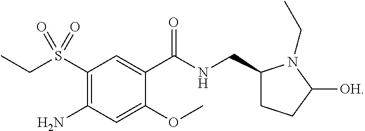 [SMILES: CCN1C(O)CC[C@H]1CNC(=O)c1cc(S(=O)(=O)CC)c(N)cc1OC]

Embodiment 134. An enantiomerically pure crystalline form of (S)-(−)-amisulpride prepared by the method of any one of embodiments 3-43, 58-78, 80-93, 96-98, 101, 102, and 110-114, comprising less than about 0.2 wt % of a compound of the following formula:

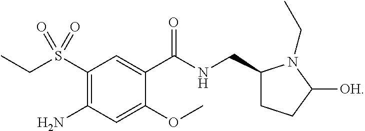 [SMILES: CCN1C(O)CC[C@H]1CNC(=O)c1cc(S(=O)(=O)CC)c(N)cc1OC]

Embodiment 135. A pharmaceutical composition comprising the enantiomerically pure crystalline form of (R)-(+)-amisulpride prepared by the method of any one of embodiments 1, 2, 5-57, 71-79, 81-95, 98-100, and 103-108, and a pharmaceutically acceptable carrier.

Embodiment 136. A pharmaceutical composition comprising the enantiomerically pure crystalline form of (S)-(−)-amisulpride prepared by the method of any one of embodiments 3-43, 58-78, 80-93, 96-98, 101, 102, and 110-114, and a pharmaceutically acceptable carrier.

Embodiment 137. A pharmaceutical composition comprising: (i) the enantiomerically pure crystalline form of (R)-(+)-amisulpride prepared by the method of any one of embodiments 1, 2, 5-57, 71-79, 81-95, 98-100, and 103-108; (ii) the enantiomerically pure crystalline form of (S)-(−)-amisulpride prepared by the method of any one of embodiments 3-43, 58-78, 80-93, 96-98, 101, 102, and 110-114; and (iii) a pharmaceutically acceptable carrier, wherein the crystalline form of (R)-(+)-amisulpride and the crystalline form of (S)-(−)-amisulpride are in a ratio of about 65:35 to about 88:12 by weight of free base.

Embodiment 138. The pharmaceutical composition of embodiment 137, wherein the crystalline form of (R)-(+)-amisulpride and the crystalline form of (S)-(−)-amisulpride are in a ratio of about 75:25 to about 88:12 by weight of free base.

Embodiment 139. The pharmaceutical composition of embodiment 137 or 138, wherein the crystalline form of (R)-(+)-amisulpride and the crystalline form of (S)-(−)-amisulpride are in a ratio of about 80:20 to about 88:12 by weight of free base.

Embodiment 140. The pharmaceutical composition of any one of embodiments 137-139, wherein the crystalline form of (R)-(+)-amisulpride and the crystalline form of (S)-(−)-amisulpride are in a ratio of about 85:15 by weight of free base.

Embodiment 141. The pharmaceutical composition of any one of embodiments 135-140, comprising less than about 1.0 wt % of a compound of the following formula:

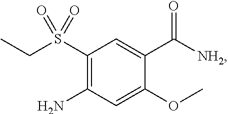 [SMILES: CCS(=O)(=O)c1cc(C(N)=O)c(OC)cc1N]

wherein the recited wt % is calculated relative to the total amount of (R)-(+)-amisulpride and (S)-(−)-amisulpride that is present in the pharmaceutical composition.

Embodiment 142. The pharmaceutical composition of any one of embodiments 135-140, comprising less than about 0.2 wt % of a compound of the following formula:

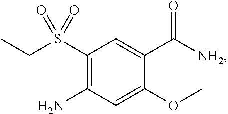 [SMILES: CCS(=O)(=O)c1cc(C(N)=O)c(OC)cc1N]

wherein the recited wt % is calculated relative to the total amount of (R)-(+)-amisulpride and (S)-(−)-amisulpride that is present in the pharmaceutical composition.

Embodiment 143. The pharmaceutical composition of any one of embodiments 135-142, comprising less than about 1.0 wt % of a compound of the following formula:

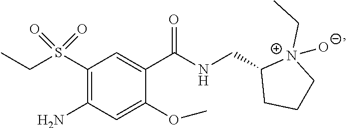 [SMILES: CC[N+]1([O-])CCC[C@@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

Embodiment 144. The pharmaceutical composition of any one of embodiments 135-142, comprising less than about 0.2 wt % of a compound of the following formula:

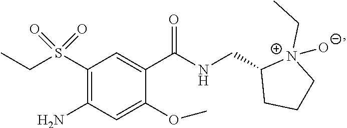 [SMILES: CC[N+]1([O-])CCC[C@@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

Embodiment 145. The pharmaceutical composition of any one of embodiments 135-144, comprising less than about 1.0 wt % of a compound of the following formula:

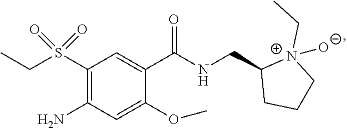 [SMILES: CC[N+]1([O-])CCC[C@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

Embodiment 146. The pharmaceutical composition of any one of embodiments 135-144, comprising less than about 0.2 wt % of a compound of the following formula:

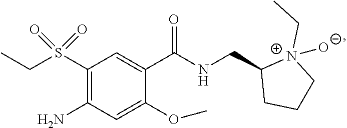 [SMILES: CC[N+]1([O-])CCC[C@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

Embodiment 147. The pharmaceutical composition of any one of embodiments 135-146, comprising less than about 1.0 wt % of a compound of the following formula:

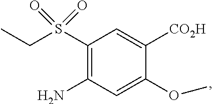 [SMILES: CCS(=O)(=O)c1cc(C(=O)O)c(OC)cc1N]

wherein the recited wt % is calculated relative to the total amount of (R)-(+)-amisulpride and (S)-(−)-amisulpride that is present in the pharmaceutical composition.

Embodiment 148. The pharmaceutical composition of any one of embodiments 135-147, comprising less than about 1.0 wt % of a compound of the following formula:

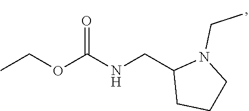

wherein the recited wt % is calculated relative to the total amount of (R)-(+)-amisulpride and (S)-(−)-amisulpride that is present in the pharmaceutical composition.

Embodiment 149. The pharmaceutical composition of any one of embodiments 135-148, comprising less than about 1.0 wt % of a compound of the following formula:

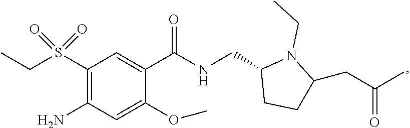 [SMILES: CCN1C(CC(C)=O)CC[C@@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

Embodiment 150. The pharmaceutical composition of any one of embodiments 135-149, comprising less than about 1.0 wt % of a compound of the following formula:

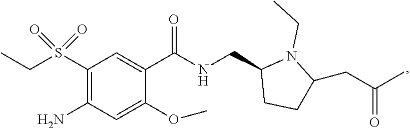 [SMILES: CCN1C(CC(C)=O)CC[C@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

Embodiment 151. The pharmaceutical composition of any one of embodiments 135-150, comprising less than about 1.0 wt % of a compound of the following formula:

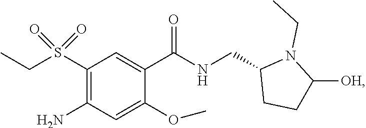 [SMILES: CCN1C(O)CC[C@@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

Embodiment 152. The pharmaceutical composition of any one of embodiments 135-151, comprising less than about 1.0 wt % of a compound of the following formula:

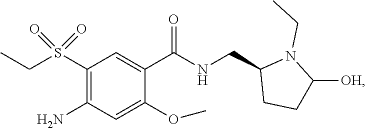 [SMILES: CCN1C(O)CC[C@H]1CNC(=O)c1cc(S(=O)(=O)CC)c(N)cc1OC]

wherein the recited wt % is calculated relative to the total amount of (R)-(+)-amisulpride and (S)-(−)-amisulpride that is present in the pharmaceutical composition.

Embodiment 153. A method of treating a psychiatric disorder in a subject comprising administering to the subject an enantiomerically pure crystalline form of (R)-(+)-amisulpride or (S)-(−)-amisulpride prepared by any one of embodiments 1-114, or an enantiomerically pure crystalline form of (R)-(+)-amisulpride or (S)-(−)-amisulpride of any one of embodiments 115-134, or a pharmaceutical composition of any one of embodiments 135-152.

Embodiment 154. The method according to embodiment 153, wherein the psychiatric disorder is a depressive order.

Embodiment 155. The method according to embodiment 153, wherein the psychiatric disorder is bipolar disorder.

Embodiment 156. The method according to embodiment 153, wherein the psychiatric disorder is bipolar depression.

Embodiment 157. The method according to embodiment 153, wherein the psychiatric disorder is major depressive disorder (MDD).

Embodiment 158. The method according to embodiment 153, wherein the psychiatric disorder is major depressive disorder with mixed features (MDD-MF).

Embodiment 159. The method according to embodiment 153, wherein the psychiatric disorder is treatment resistant depression (TRD).

Embodiment 160. The method according to embodiment 153, wherein the psychiatric disorder is schizophrenia.

Embodiment 161. An enantiomerically pure crystalline form of (R)-(+)-amisulpride prepared by the method of any one of embodiments 1, 2, 5-57, 71-79, 81-95, 98-100, and 103-108, wherein the crystalline form of (R)-(+)-amisulpride has a greater than about 95% chemical purity and about 95% enantiomeric purity.

Embodiment 162. An enantiomerically pure crystalline form of (R)-(+)-amisulpride prepared by the method of any one of embodiments 1, 2, 5-57, 71-79, 81-95, 98-100, and 103-108, wherein the crystalline form of (R)-(+)-amisulpride has a greater than about 98% chemical purity and about 98% enantiomeric purity.

Embodiment 163. An enantiomerically pure crystalline form of (R)-(+)-amisulpride prepared by the method of any one of embodiments 1, 2, 5-57, 71-79, 81-95, 98-100, and 103-108, wherein the crystalline form of (R)-(+)-amisulpride has a greater than about 99% chemical purity and about 99% enantiomeric purity.

Embodiment 164. An enantiomerically pure crystalline form of (S)-(−)-amisulpride prepared by the method of any one of embodiments 3-43, 58-78, 80-93, 96-98, 101, 102, and 110-114, wherein the crystalline form of (S)-(−)-amisulpride has a greater than about 95% chemical purity and about 95% enantiomeric purity.

Embodiment 165. An enantiomerically pure crystalline form of (S)-(−)-amisulpride prepared by the method of any one of embodiments 3-43, 58-78, 80-93, 96-98, 101, 102, and 110-114, wherein the crystalline form of (S)-(−)-amisulpride has a greater than about 98% chemical purity and about 98% enantiomeric purity.

Embodiment 166. An enantiomerically pure crystalline form of (S)-(−)-amisulpride prepared method of any one of embodiments 3-43, 58-78, 80-93, 96-98, 101, 102, and 110-114, wherein the crystalline form of (S)-(−)-amisulpride has a greater than about 99% chemical purity and about 99% enantiomeric purity.

Although the invention has been described with reference to a specific embodiments this description is not meant to be construed in a limiting sense. The invention being thus described, it is apparent that the same can be varied in many ways. Such variations are not to be regarded as a departure from the spirit and scope of the present invention, and all such modifications, alternatives, and equivalents as would be apparent to those skilled in the art are intended to be included within the scope of the appended claims.

What is claimed is:

1. A method of preparing an enantiomerically pure crystalline form of (R)-(+)-amisulpride comprising the steps of:
(a) coupling, in the presence of a tertiary amine and an acid activating reagent, 4-amino-5-(ethylsulfonyl)-2-methoxybenzoic acid and (R)-(1-ethylpyrrolidin-2-yl) methanamine to form a reaction mixture; and
(b) isolating from the reaction mixture of step (a), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°;
wherein step (b) does not comprise formation of a solvate of (R)-(+)-amisulpride.

2. The method of claim 1, wherein the coupling of step (a) comprises the steps of:
(a1) reacting 4-amino-5-(ethylsulfonyl)-2-methoxybenzoic acid with the tertiary amine and the acid activating reagent to form a first reaction mixture; and
(a2) adding (R)-(1-ethylpyrrolidin-2-yl)methanamine to the first reaction mixture.

3. A method of preparing an enantiomerically pure crystalline form of (S)-(−)-amisulpride comprising the steps of:
(a) coupling, in the presence of a tertiary amine and an acid activating reagent, 4-amino-5-(ethylsulfonyl)-2-methoxybenzoic acid and (S)-(1-ethylpyrrolidin-2-yl) methanamine to form a reaction mixture; and
(b) isolating from the reaction mixture of step (a), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°;
wherein the step (b) does not comprise formation of a solvate of (S)-(−)-amisulpride.

4. The method of claim 3, wherein the coupling of step (a) comprises the steps of:
(a1) reacting 4-amino-5-(ethylsulfonyl)-2-methoxybenzoic acid with the tertiary amine and the acid activating reagent to form a first reaction mixture; and
(a2) adding (S)-(1-ethylpyrrolidin-2-yl)methanamine to the first reaction mixture.

5. The method of any one of claims 1-4, wherein step (b) comprises step (b1): concentrating the reaction mixture of step (a) to give a mixture of step (b1).

6. The method of claim 5, further comprising step (b2): treating the mixture of step (b1) with a base and the isolation reagent to form a step (b2) mixture.

7. The method of claim 6, wherein the base is an aqueous solution of an inorganic base.

8. The method of claim 7, wherein the base is an aqueous solution of potassium carbonate.

9. The method of any one of claims 6-8, further comprising step (b3): separating the step (b2) mixture to obtain an organic phase.

10. The method of claim 9, further comprising step (b4): concentrating the organic phase to a first concentrated solution having less than about 2 wt % water.

11. The method of claim 10, wherein the first concentrated solution has about 1.0 wt % to about 0.001 wt % water.

12. The method of claim 11, wherein the first concentrated solution has about 1.0 wt % to about 0.01 wt % water.

13. The method of claim 12, wherein the first concentrated solution has about 0.5 wt % to about 0.01 wt % water.

14. The method of any one of claims 10-13, further comprising step (b5): concentrating the first concentrated solution to a second concentrated solution, wherein the second concentrated solution has a total weight of (R)-(+)-amisulpride or (S)-(−)-amisulpride that is about 15 wt % to about 65 wt % of the second concentrated solution.

15. The method of claim 14, wherein the second concentrated solution has a total weight of (R)-(+)-amisulpride or (S)-(−)-amisulpride that is about 35 wt % to about 40 wt % of the second concentrated solution.

16. The method of claim 14, wherein the second concentrated solution has a total weight of (R)-(+)-amisulpride or (S)-(−)-amisulpride that is about 33 wt % to about 38 wt % of the second concentrated solution.

17. The method of claim 14, wherein the second concentrated solution has a total weight of (R)-(+)-amisulpride or (S)-(−)-amisulpride that is about 35 wt % of the second concentrated solution.

18. The method of any one of claims 14-17, further comprising adding S5, wherein S5 is a solvent, to the second concentrated solution.

19. The method of claim 18, wherein S5 is methyl tert-butyl ether.

20. The method of any one of claims 14-19, further comprising step (b6): adding a seed amount of a crystalline form of (R)-(+)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2° to the second concentrated solution to form a seeded mixture.

21. The method of any one of claims 14-19, further comprising step (b6): adding a seed amount of a crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2° to the second concentrated solution to form a seeded mixture.

22. The method of claim 20 or 21, wherein the seed amount has a total weight that is about 0.001 wt % to about 15 wt % of the second concentrated solution.

23. The method of claim 20 or 21, wherein the seed amount has a total weight that is about 0.1 wt % to about 10 wt % of the second concentrated solution.

24. The method of claim 20 or 21, wherein the seed amount has a total weight that is about 0.1 wt % to about 5 wt % of the expected yield of the (R)-(+)-amisulpride or (S)-(−)-amisulpride present in the second concentrated solution.

25. The method of claim 20 or 21, wherein the seed amount has a total weight that is about 0.1 wt % to about 2.0 wt % of the expected yield of the (R)-(+)-amisulpride or (S)-(−)-amisulpride present in the second concentrated solution.

26. The method of claim 20 or 21, wherein the seed amount has a total weight that is about 0.4 wt %, about 1.4 wt %, or about 2.0 wt % of the expected yield of the (R)-(+)-amisulpride or (S)-(−)-amisulpride present in the second concentrated solution.

27. The method of claim 20 or 21, wherein the seed amount has a total weight that is about 0.75 wt % of the expected yield of the (R)-(+)-amisulpride or (S)-(−)-amisulpride present in the second concentrated solution.

28. The method of any one of claims 20-27, further comprising step (b7): filtering the seeded mixture of step (b6) to obtain a product solid.

29. The method of claim 28, further comprising step (b8): drying the product solid to obtain a crude product.

30. The method of claim 29, further comprising step (b9): recrystallizing the crude product in the presence of the isolation reagent.

31. The method of claim 30, wherein the recrystallizing comprises (i) dissolving the crude product with the isolation reagent to form a recrystallization solution, (ii) filtering and concentrating the recrystallization solution, and (iii) adding a seed amount of a crystalline form of (R)-(+)-amisulpride or a crystalline form of (S)-(−)-amisulpride.

32. The method of claim 30, wherein the recrystallizing comprises (i) heating the crude product in the presence of an isolation reagent at a first elevated temperature to form a recrystallization solution, (ii) filtering and concentrating the recrystallization solution to form a concentrated recrystallization solution; (iii) adding S5, wherein S5 is a solvent, to the concentrated recrystallization solution; (iv) adding a seed amount of a crystalline form of (R)-(+)-amisulpride or a crystalline form of (S)-(−)-amisulpride to the concentrated recrystallization solution of step (iii) to form a seeded recrystallization solution; and (v) cooling the seeded recrystallization solution.

33. The method of claim 32, wherein the first elevated temperature is from about 50° C. to about 55° C.

34. The method of claim 32 or 33, wherein the cooling comprises cooling to a first reduced temperature over a first period of time, and then cooling to a second reduced temperature over a second period of time.

35. The method of claim 34, wherein the first reduced temperature is from about 38° C. to about 42° C.

36. The method of claim 34 or 35, wherein the first period of time is about 120 min.

37. The method of any one of claims 34-36, wherein the second reduced temperature is from about 10° C. to about 15° C.

38. The method of any one of claims 34-37, wherein the second period of time is about 60 min.

39. The method of any one of claims 34-38, wherein the cooling over the first period of time is performed at a first cooling rate.

40. The method of any one of claims 34-39, wherein the cooling over the second period of time is performed at a second cooling rate.

41. The method of claim 40, wherein the second cooling rate is greater than the first cooling rate.

42. The method of claim 39, wherein the first cooling rate is about 0.1° C./min.

43. The method of claim 40, wherein the second cooling rate is about 0.5° C./min.

44. A method of preparing an enantiomerically pure crystalline form of (R)-(+)-amisulpride comprising the steps of:
(a) reacting 4-amino-5-(ethylsulfonyl)-2-methoxybenzoic acid with a tertiary amine and an acid activating reagent to form a reaction mixture;
(b) adding a (R)-(1-ethylpyrrolidin-2-yl)methanamine salt to the reaction mixture to form a mixture of step (b); and
(c) isolating from the mixture of step (b), in the presence of an isolation reagent, an enantiomerically pure crystalline form of (R)-(+)-amisulpride characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2°.

45. The method of claim 44, wherein step (c) does not comprise formation of a solvate of (R)-(+)-amisulpride.

46. The method of claim 44 or 45, wherein the isolating of step (c) comprises
(d1) adding the isolation reagent to the mixture of step (b), followed by an aqueous base solution to form a mixture of step (d1),
(d2) separating the mixture of step (d1) to obtain an organic phase;
(d3) concentrating the organic phase of step (d2) to produce a product solution having about 5.0 wt % to about 0.001 wt % water;
(d4) adding to the product solution a seed amount of a crystalline form of (R)-(+)-amisulpride, characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (R)-(+)-amisulpride.

47. The method of any one of claims 44-46, further comprising recrystallizing the enantiomerically pure crystalline form of (R)-(+)-amisulpride of step (d6).

48. The method of claim 47, wherein the recrystallizing comprises (i) heating the enantiomerically pure crystalline form of (R)-(+)-amisulpride of step (d6) in the presence of an isolation reagent at a first elevated temperature to form a recrystallization solution, (ii) filtering and concentrating the recrystallization solution to form a concentrated recrystallization solution; (iii) adding S5, wherein S5 is a solvent, to the concentrated recrystallization solution; (iv) adding a seed amount of a crystalline form of (R)-(+)-amisulpride to the concentrated recrystallization solution of step (iii) to form a seeded recrystallization solution; and (v) cooling the seeded recrystallization solution.

49. The method of claim 48, wherein the first elevated temperature is from about 50° C. to about 55° C.

50. The method of claim 48 or 49, wherein the cooling comprises cooling to a first reduced temperature over a first period of time, and then cooling to a second reduced temperature over a second period of time.

51. The method of claim 50, wherein the first reduced temperature is from about 38° C. to about 42° C.

52. The method of claim 50 or 51, wherein the first period of time is about 120 min.

53. The method of any one of claims 50-52, wherein the second reduced temperature is from about 10° C. to about 15° C.

54. The method of any one of claims 50-53, wherein the second period of time is about 60 min.

55. The method of any one of claims 48-54, wherein S5 is an ether solvent.

56. The method of any one of claims 48-54, wherein S5 is methyl tert-butyl ether.

57. The method of any one of claims 48-56, wherein the method further comprises isolating and drying the crystalline form of (R)-(+)-amisulpride.

58. A method of preparing an enantiomerically pure crystalline form of (S)-(−)-amisulpride comprising the steps of:
(a) reacting 4-amino-5-(ethylsulfonyl)-2-methoxybenzoic acid with a tertiary amine and an acid activating reagent to form a reaction mixture;
(b) adding a (S)-(1-ethylpyrrolidin-2-yl)methanamine salt to the reaction mixture of step (a) to form a mixture of step (b); and
(c) isolating from the mixture of step (b), in the presence of an isolation reagent, an enantiomerically pure crystalline form of (S)-(−)-amisulpride characterized by an x-ray powder diffraction pattern (XRPD) comprising, when measured using CuKα radiation, at least approximate peak positions (2θ), at least at 7.0±0.2° and 9.7±0.2°, and further peaks at 15.4±0.2° and/or 19.4±0.2°.

59. The method of claim 58, wherein step (c) does not comprise formation of a solvate of (S)-(−)-amisulpride.

60. The method of claim 58 or 59, wherein the isolating of step (c) comprises:
(d1) adding the isolation reagent to the mixture of step (b), followed by an aqueous base solution to form a mixture of step (d1);
(d2) separating the mixture of step (d1) to obtain an organic phase;
(d3) concentrating the organic phase of step (d2) to produce a product solution with about 5.0 wt % to about 0.01 wt % water;
(d4) adding to the product solution of step (d3) with a seed amount of a crystalline form of (S)-(−)-amisulpride characterized by an x-ray powder diffraction pattern (XRPD) comprising, when measured using CuKα radiation, at least approximate peak positions (2θ), at 7.0±0.2° and 9.7±0.2°, and further peaks at 15.4±0.2° and/or 19.4±0.2° to form a seeded mixture;
(d5) filtering the seeded mixture of step (d4) to obtain a product solid; and
(d6) drying the product solid of step (d5) to produce the enantiomerically pure crystalline form of (S)-(−)-amisulpride.

61. The method of any one of claims 58-60, further comprising recrystallizing the enantiomerically pure crystalline form of (S)-(−)-amisulpride of step (d6).

62. The method of claim 61, wherein the recrystallizing comprises (i) heating the enantiomerically pure crystalline form of (S)-(−)-amisulpride of step (d6) in the presence of an isolation reagent at a first elevated temperature to form a recrystallization solution, (ii) filtering and concentrating the recrystallization solution to form a concentrated recrystallization solution; (iii) adding S5, wherein S5 is a solvent, to the concentrated recrystallization solution; (iv) adding a seed amount of a crystalline form of (S)-(−)-amisulpride to the concentrated recrystallization solution of step (iii) to form a seeded recrystallization solution; and (v) cooling the seeded recrystallization solution.

63. The method of claim 61 or 62, wherein the first elevated temperature is from about 50° C. to about 55° C.

64. The method of claim 61 or 62, wherein the cooling comprises cooling to a first reduced temperature over a first period of time, and then cooling to a second reduced temperature over a second period of time.

65. The method of claim 64, wherein the first reduced temperature is from about 38° C. to about 42° C.

66. The method of claim 64 or 65, wherein the first period of time is about 120 min.

67. The method of any one of claims 64-66, wherein the second reduced temperature is from about 10° C. to about 15° C.

68. The method of any one of claims 64-67, wherein the second period of time is about 60 min.

69. The method of any one of claims 62-68, wherein S5 is an ether solvent.

70. The method of any one of claims 62-68, wherein S5 is methyl tert-butyl ether.

71. The method of claim 46 or 60, wherein the product solution of step (d3) has about 0.001 wt % to about 0.5 wt % water.

72. The method of any one of claims 1-71, wherein the tertiary amine is of the formula 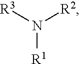
wherein:
(i) R¹, R² and R³ are each independently C₁₋₆ alkyl, C₃₋₆ cycloalkyl, 3-10 membered monocyclic or bicyclic heterocycloalkyl, or 5-10 membered monocyclic heteroaryl; or
(ii) R¹ is C₁₋₆ alkyl, C₃₋₆ cycloalkyl, 3-10 membered monocyclic or bicyclic heterocycloalkyl, or 5-10 membered monocyclic heteroaryl, and R² and R³ together with the N atom to which they are attached form a 3-10 membered monocyclic or bicyclic heterocycloalkyl or a 5-10 membered monocyclic heteroaryl.

73. The method of claim 72, wherein R² and R³ together with the N atom to which they are attached form a 3-10 membered monocyclic or bicyclic heterocycloalkyl or a 5-10 membered monocyclic heteroaryl.

74. The method of any one of claims 1-73, wherein the tertiary amine is triethyl amine.

75. The method of any one of claims 1-73, wherein the tertiary amine is 4-methylmorpholine.

76. The method of any one of claims 1-75, wherein the acid activating reagent is of the formula 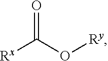
wherein R^{x} is halogen and R^{y} is C₁₋₅ alkyl.

77. The method of any one of claims 1-75, wherein the acid activating reagent is ethyl chloroformate.

78. The method of any one of claims 1-77, wherein the isolation reagent is of the formula 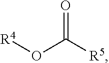
wherein R⁴ is C₁₋₆ alkyl; and R⁵ is C₁₋₅ alkyl or C₁₋₅ alkoxide.

79. A method of preparing an enantiomerically pure crystalline form of (R)-(+)-amisulpride comprising the steps of:
(a) heating (R)-(+)-amisulpride in the presence of an isolation reagent to a first elevated temperature to form a crystallization mixture;
(b) adding a seed amount of Form A of (R)-(+)-amisulpride to the crystallization mixture to form a seeded mixture;
(c) cooling the seeded mixture to a first reduced temperature over a first period of time;
(d) cooling the seeded mixture from step (c) to a second reduced temperature over a second period of time; and
(e) filtering the seeded mixture from step (d) to provide an enantiomerically pure crystalline form of (R)-(+)-amisulpride characterized by an x-ray powder diffraction pattern (XRPD) comprising, when measured using CuKα, radiation, at least approximate peak positions (2θ) at 7.0±0.2° and 9.7±0.2°, and further peaks at 15.4±0.2° and/or 19.4±0.2°.

80. A method of preparing an enantiomerically pure crystalline form of (S)-(−)-amisulpride comprising the steps of:
(a) heating (S)-(−)-amisulpride in the presence of an isolation reagent to a first elevated temperature to form a crystallization mixture;
(b) adding a seed amount of Form A′ of (S)-(−)-amisulpride to the crystallization mixture to form a seeded mixture;
(c) cooling the seeded mixture to a first reduced temperature over a first period of time;
(d) cooling the seeded mixture from step (c) to a second reduced temperature over a second period of time; and
(e) filtering the seeded mixture from step (d) to provide an enantiomerically pure crystalline form of (S)-(−)-amisulpride characterized by an x-ray powder diffraction pattern (XRPD) comprising, when measured using CuKα radiation, at least approximate peak positions (2θ) at 7.0±0.2° and 9.7±0.2°, and further peaks at 15.4±0.2° and/or 19.4±0.2°.

81. The method of claim 79 or 80, wherein the first elevated temperature is from about 50° C. to about 55° C.

82. The method of any one of claims 79-81, wherein the first reduced temperature is from about 38° C. to about 42° C.

83. The method of any one of claims 79-82, wherein the first period of time is about 120 min.

84. The method of any one of claims 79-83, wherein the second reduced temperature is from about 10° C. to about 15° C.

85. The method of any one of claims 79-84, wherein the second period of time is about 60 min.

86. The method of any one of claims 79-85, wherein the heating of step (a) is further performed in the presence of S5, wherein S5 is an ether solvent.

87. The method of claim 86, wherein S5 is methyl tert-butyl ether.

88. The method of any one of claims 1-87, wherein the isolation reagent is of the formula 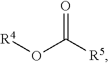
wherein R⁴ is C₃₋₆ alkyl; and R⁵ is C₁₋₅ alkyl.

89. The method of any one of claims 1-88, wherein the isolation reagent is not ethyl acetate.

90. The method of any one of claims 1-88, wherein the isolation reagent is isopropyl acetate.

91. The method of any one of claims 1-87, wherein the isolation reagent is of the formula 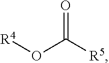
wherein R⁴ is C₁₋₆ alkyl; and R⁵ is C₁₋₅ alkoxide.

92. The method of any one of claims 1-87, wherein the isolation reagent is diethyl carbonate or dimethyl carbonate.

93. The method of any one of claims 1-87, wherein the isolation reagent is of the formula R⁶COR⁷, where each of R⁶ and R⁷ is independently C₁₋₅ alkyl.

94. The method of any one of claims 44-57, 71-78, and 88-93, wherein the (R)-(1-ethylpyrrolidin-2-yl)methanamine salt is a bis tartrate salt of (R)-(1-ethylpyrrolidin-2-yl)methanamine.

95. The method of any one of claims 44-57, 71-78, and 88-93, wherein the (R)-(1-ethylpyrrolidin-2-yl)methanamine salt is a bis L-tartrate salt of (R)-(1-ethylpyrrolidin-2-yl)methanamine.

96. The method of any one of claims 58-78 and 88-93, wherein the (S)-(1-ethylpyrrolidin-2-yl)methanamine salt is a bis tartrate salt of (S)-(1-ethylpyrrolidin-2-yl)methanamine.

97. The method of any one claims 58-78 and 88-93, wherein the (S)-(1-ethylpyrrolidin-2-yl)methanamine salt is a bis D-tartrate salt of (S)-(1-ethylpyrrolidin-2-yl)methanamine.

98. The method of any one of claims 1-87, further comprising recrystallizing the enantiomerically pure crystalline form of (R)-(+)-amisulpride or the enantiomerically pure crystalline form of (S)-(−)-amisulpride in the presence of isopropyl acetate.

99. The method of any one of claims 20, 22-43, 46-57, 71-78, 79, 81-95, and 98, wherein the seed amount of a crystalline form of (R)-(+)-amisulpride has a greater than about 95% chemical purity and a greater than about 95% enantiomeric purity.

100. The method of any one of claims 20, 22-43, 46-57, 71-78, 79, 81-95, and 98, wherein the seed amount of a crystalline form of (R)-(+)-amisulpride has a greater than about 98% chemical purity and a greater than about 98% enantiomeric purity.

101. The method of any one of claims 21-43, 60-78, 80-93, and 96-98, wherein the seed amount of a crystalline form of (S)-(−)-amisulpride has a greater than about 95% chemical purity and a greater than about 95% enantiomeric purity.

102. The method of any one of claims 21-43, 60-78, 80-93, and 96-98, wherein the seed amount of a crystalline form of (S)-(−)-amisulpride has a greater than about 98% chemical purity and a greater than about 98% enantiomeric purity.

103. The method of any one of claims 1, 2, 5-57, 71-79, 81-95, and 98-100, wherein the enantiomerically pure crystalline form of (R)-(+)-amisulpride has a greater than about 95% chemical purity.

104. The method of any one of claims 1, 2, 5-57, 71-79, 81-95, and 98-100, wherein the enantiomerically pure crystalline form of (R)-(+)-amisulpride has a greater than about 98% chemical purity.

105. The method of any one of claims 1, 2, 5-57, 71-79, 81-95, and 98-100, wherein the enantiomerically pure crystalline form of (R)-(+)-amisulpride has a greater than about 99% chemical purity.

106. The method of any one of claims 1, 2, 5-57, 71-79, 81-95, and 98-100, wherein the enantiomerically pure crystalline form of (R)-(+)-amisulpride has a greater than about 95% enantiomeric purity.

107. The method of any one of claims 1, 2, 5-57, 71-79, 81-95, and 98-100, wherein the enantiomerically pure crystalline form of (R)-(+)-amisulpride has a greater than about 98% enantiomeric purity.

108. The method of any one of claims 1, 2, 5-57, 71-79, 81-95, and 98-100, wherein the enantiomerically pure crystalline form of (R)-(+)-amisulpride has a greater than about 99% enantiomeric purity.

109. The method of any one of claims 3-43, 58-78, 80-93, 96-98, 101, and 102, wherein the enantiomerically pure crystalline form of (S)-(−)-amisulpride has a greater than about 95% chemical purity.

110. The method of any one of claims 3-43, 58-78, 80-93, 96-98, 101, and 102, wherein the enantiomerically pure crystalline form of (S)-(−)-amisulpride has a greater than about 98% chemical purity.

111. The method of any one of claims 3-43, 58-78, 80-93, 96-98, 101, and 102, wherein the enantiomerically pure crystalline form of (S)-(−)-amisulpride has a greater than about 99% chemical purity.

112. The method of any one of claims 3-43, 58-78, 80-93, 96-98, 101, and 102, wherein the enantiomerically pure crystalline form of (S)-(−)-amisulpride has a greater than about 95% enantiomeric purity.

113. The method of any one of claims 3-43, 58-78, 80-93, 96-98, 101, and 102, wherein the enantiomerically pure crystalline form of (S)-(−)-amisulpride has a greater than about 98% enantiomeric purity.

114. The method of any one of claims 3-43, 58-78, 80-93, 96-98, 101, and 102, wherein the enantiomerically pure crystalline form of (S)-(−)-amisulpride has a greater than about 99% enantiomeric purity.

115. An enantiomerically pure crystalline form of (R)-(+)-amisulpride, wherein the crystalline form of (R)-(+)-amisulpride has a particle size distribution D₁₀ value of about 2 to about 10 μm; a particle size distribution D₅₀ value of about 15 to about 30 μm; and a particle size distribution D₉₀ value of about 75 to about 100 μm, as measured by a laser diffraction particle size analyzer.

116. An enantiomerically pure crystalline form of (S)-(−)-amisulpride, wherein the crystalline form of (S)-(−)-amisulpride has a particle size distribution D₁₀ value of about 2 to about 10 μm; a particle size distribution D₅₀ value of about 15 to about 30 μm; and a particle size distribution D₉₀ value of about 75 to about 100 μm, as measured by a laser diffraction particle size analyzer.

117. An enantiomerically pure crystalline form of (R)-(+)-amisulpride or (S)-(−)-amisulpride prepared by the method of any one of claims 1-114, comprising less than about 1.0 wt % of a compound of the following formula:

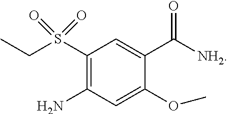 [SMILES: CCS(=O)(=O)c1cc(C(N)=O)c(OC)cc1N]

118. An enantiomerically pure crystalline form of (R)-(+)-amisulpride or (S)-(−)-amisulpride prepared by the method of any one of claims 1-114, comprising less than about 0.2 wt % of a compound of the following formula:

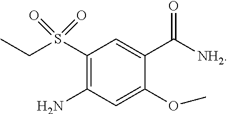 [SMILES: CCS(=O)(=O)c1cc(C(N)=O)c(OC)cc1N]

119. An enantiomerically pure crystalline form of (R)-(+)-amisulpride or (S)-(−)-amisulpride prepared by the method of any one of claims 1-114, comprising less than about 1.0 wt % of a compound of the following formula:

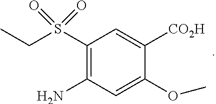 [SMILES: CCS(=O)(=O)c1cc(C(=O)O)c(OC)cc1N]

120. An enantiomerically pure crystalline form of (R)-(+)-amisulpride or (S)-(−)-amisulpride prepared by the method of any one of claims 1-114, comprising less than about 0.2 wt % of a compound of the following formula:

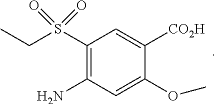 [SMILES: CCS(=O)(=O)c1cc(C(=O)O)c(OC)cc1N]

121. An enantiomerically pure crystalline form of (R)-(+)-amisulpride or (S)-(−)-amisulpride prepared by the method of any one of claims 1-114, comprising less than about 1.0 wt % of a compound of the following formula:

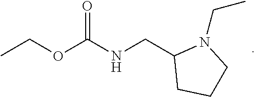

122. An enantiomerically pure crystalline form of (R)-(+)-amisulpride or (S)-(−)-amisulpride prepared by the method of any one of claims 1-114, comprising less than about 0.2 wt % of a compound of the following formula:

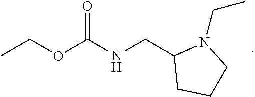

123. An enantiomerically pure crystalline form of (R)-(+)-amisulpride prepared by the method of any one of claims 1, 2, 5-57, 71-79, 81-95, 98-100, and 103-108, comprising less than about 1.0 wt % of a compound of the following formula:

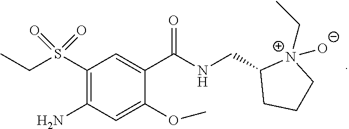 [SMILES: CC[N+]1([O-])CCC[C@@H]1CNC(=O)c1cc(S(=O)(=O)CC)c(N)cc1OC]

124. An enantiomerically pure crystalline form of (R)-(+)-amisulpride prepared by the method of any one of claims 1, 2, 5-57, 71-79, 81-95, 98-100, and 103-108, comprises less than about 0.2 wt % of a compound of the following formula:

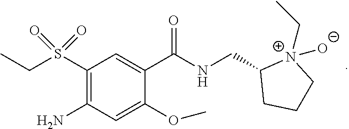 [SMILES: CC[N+]1([O-])CCC[C@@H]1CNC(=O)c1cc(S(=O)(=O)CC)c(N)cc1OC]

125. An enantiomerically pure crystalline form of (R)-(+)-amisulpride prepared by the method of any one of claims 1, 2, 5-57, 71-79, 81-95, 98-100, and 103-108, comprising less than about 1.0 wt % of a compound of the following formula:

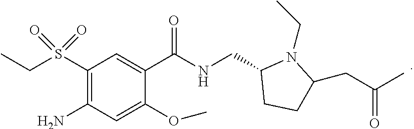 [SMILES: CCN1C(CC(C)=O)CC[C@@H]1CNC(=O)c1cc(S(=O)(=O)CC)c(N)cc1OC]

126. An enantiomerically pure crystalline form of (R)-(+)-amisulpride prepared by the method of any one of claims 1, 2, 5-57, 71-79, 81-95, 98-100, and 103-108, comprises less than about 0.2 wt % of a compound of the following formula:

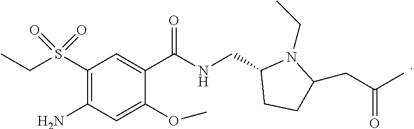 [SMILES: CCN1C(CC(C)=O)CC[C@@H]1CNC(=O)c1cc(S(=O)(=O)CC)c(N)cc1OC]

127. An enantiomerically pure crystalline form of (R)-(+)-amisulpride prepared by the method of any one of claims 1, 2, 5-57, 71-79, 81-95, 98-100, and 103-108, comprising less than about 1.0 wt % of a compound of the following formula:

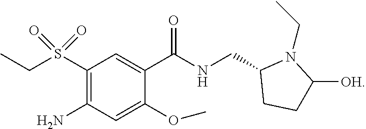 [SMILES: CCN1C(O)CC[C@@H]1CNC(=O)c1cc(S(=O)(=O)CC)c(N)cc1OC]

128. An enantiomerically pure crystalline form of (R)-(+)-amisulpride prepared by the method of any one of claims 1, 2, 5-57, 71-79, 81-95, 98-100, and 103-108, comprises less than about 0.2 wt % of a compound of the following formula:

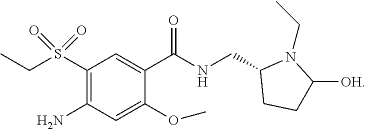 [SMILES: CCN1C(O)CC[C@@H]1CNC(=O)c1cc(S(=O)(=O)CC)c(N)cc1OC]

129. An enantiomerically pure crystalline form of (S)-(−)-amisulpride prepared by the method of any one of claims 3-43, 58-78, 80-93, 96-98, 101, 102, and 110-114, comprising less than about 1.0 wt % of a compound of the following formula:

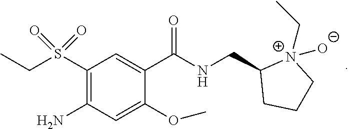 [SMILES: CC[N+]1([O-])CCC[C@H]1CNC(=O)c1cc(S(=O)(=O)CC)c(N)cc1OC]

130. An enantiomerically pure crystalline form of (S)-(−)-amisulpride prepared by the method of any one of claims 3-43, 58-78, 80-93, 96-98, 101, 102, and 110-114, comprising less than about 0.2 wt % of a compound of the following formula:

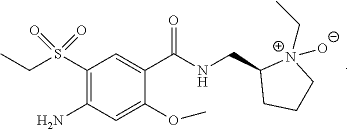 [SMILES: CC[N+]1([O-])CCC[C@H]1CNC(=O)c1cc(S(=O)(=O)CC)c(N)cc1OC]

131. An enantiomerically pure crystalline form of (S)-(−)-amisulpride prepared by the method of any one of claims 3-43, 58-78, 80-93, 96-98, 101, 102, and 110-114, comprising less than about 1.0 wt % of a compound of the following formula:

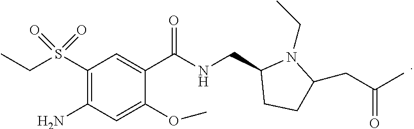 [SMILES: CCN1C(CC(C)=O)CC[C@H]1CNC(=O)c1cc(S(=O)(=O)CC)c(N)cc1OC]

132. An enantiomerically pure crystalline form of (S)-(−)-amisulpride prepared by the method of any one of claims 3-43, 58-78, 80-93, 96-98, 101, 102, and 110-114, comprising less than about 0.2 wt % of a compound of the following formula:

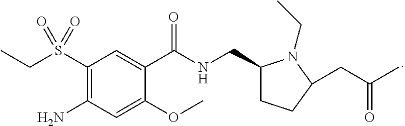 [SMILES: CCN1C(CC(C)=O)CC[C@H]1CNC(=O)c1cc(S(=O)(=O)CC)c(N)cc1OC]

133. An enantiomerically pure crystalline form of (S)-(−)-amisulpride prepared by the method of any one of claims 3-43, 58-78, 80-93, 96-98, 101, 102, and 110-114, comprising less than about 1.0 wt % of a compound of the following formula:

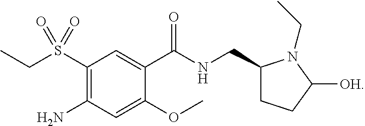 [SMILES: CCN1C(O)CC[C@H]1CNC(=O)c1cc(S(=O)(=O)CC)c(N)cc1OC]

134. An enantiomerically pure crystalline form of (S)-(−)-amisulpride prepared by the method of any one of claims 3-43, 58-78, 80-93, 96-98, 101, 102, and 110-114, comprising less than about 0.2 wt % of a compound of the following formula:

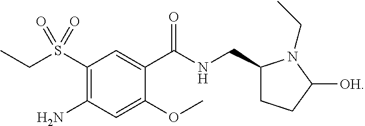 [SMILES: CCN1C(O)CC[C@H]1CNC(=O)c1cc(S(=O)(=O)CC)c(N)cc1OC]

135. A pharmaceutical composition comprising the enantiomerically pure crystalline form of (R)-(+)-amisulpride prepared by the method of any one of claims 1, 2, 5-57, 71-79, 81-95, 98-100, and 103-108, and a pharmaceutically acceptable carrier.

136. A pharmaceutical composition comprising the enantiomerically pure crystalline form of (S)-(−)-amisulpride prepared by the method of any one of claims 3-43, 58-78, 80-93, 96-98, 101, 102, and 110-114, and a pharmaceutically acceptable carrier.

137. A pharmaceutical composition comprising: (i) the enantiomerically pure crystalline form of (R)-(+)-amisulpride prepared by the method of any one of claims 1, 2, 5-57, 71-79, 81-95, 98-100, and 103-108; (ii) the enantiomerically pure crystalline form of (S)-(−)-amisulpride prepared by the method of any one of claims 3-43, 58-78, 80-93, 96-98, 101, 102, and 110-114; and (iii) a pharmaceutically acceptable carrier, wherein the crystalline form of (R)-(+)-amisulpride and the crystalline form of (S)-(−)-amisulpride are in a ratio of about 65:35 to about 88:12 by weight of free base.

138. The pharmaceutical composition of claim 137, wherein the crystalline form of (R)-(+)-amisulpride and the crystalline form of (S)-(−)-amisulpride are in a ratio of about 75:25 to about 88:12 by weight of free base.

139. The pharmaceutical composition of claim 137 or 138, wherein the crystalline form of (R)-(+)-amisulpride and the crystalline form of (S)-(−)-amisulpride are in a ratio of about 80:20 to about 88:12 by weight of free base.

140. The pharmaceutical composition of any one of claims 137-139, wherein the crystalline form of (R)-(+)-amisulpride and the crystalline form of (S)-(−)-amisulpride are in a ratio of about 85:15 by weight of free base.

141. The pharmaceutical composition of any one of claims 135-140, comprising less than about 1.0 wt % of a compound of the following formula: 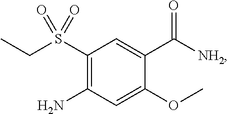 [SMILES: CCS(=O)(=O)c1cc(C(N)=O)c(OC)cc1N]
wherein the recited wt % is calculated relative to the total amount of (R)-(+)-amisulpride and (S)-(−)-amisulpride that is present in the pharmaceutical composition.

142. The pharmaceutical composition of any one of claims 135-140, comprising less than about 0.2 wt % of a compound of the following formula: 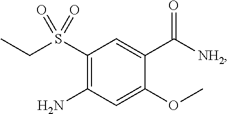 [SMILES: CCS(=O)(=O)c1cc(C(N)=O)c(OC)cc1N]
wherein the recited wt % is calculated relative to the total amount of (R)-(+)-amisulpride and (S)-(−)-amisulpride that is present in the pharmaceutical composition.

143. The pharmaceutical composition of any one of claims 135-142, comprising less than about 1.0 wt % of a compound of the following formula: 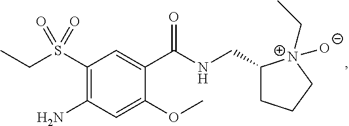 [SMILES: CC[N+]1([O-])CCC[C@@H]1CNC(=O)c1cc(S(=O)(=O)CC)c(N)cc1OC]
wherein the recited wt % is calculated relative to the total amount of (R)-(+)-amisulpride and (S)-(−)-amisulpride that is present in the pharmaceutical composition.

144. The pharmaceutical composition of any one of claims 135-142, comprising less than about 0.2 wt % of a compound of the following formula: 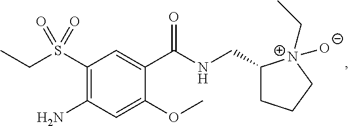 [SMILES: CC[N+]1([O-])CCC[C@@H]1CNC(=O)c1cc(S(=O)(=O)CC)c(N)cc1OC]
wherein the recited wt % is calculated relative to the total amount of (R)-(+)-amisulpride and (S)-(−)-amisulpride that is present in the pharmaceutical composition.

145. The pharmaceutical composition of any one of claims 135-144, comprising less than about 1.0 wt % of a compound of the following formula: 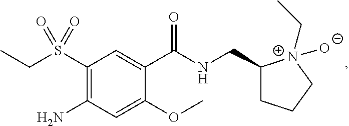 [SMILES: CC[N+]1([O-])CCC[C@H]1CNC(=O)c1cc(S(=O)(=O)CC)c(N)cc1OC]
wherein the recited wt % is calculated relative to the total amount of (R)-(+)-amisulpride and (S)-(−)-amisulpride that is present in the pharmaceutical composition.

146. The pharmaceutical composition of any one of claims 135-144, comprising less than about 0.2 wt % of a compound of the following formula: 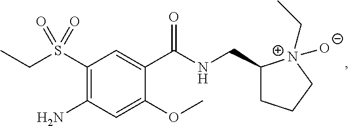 [SMILES: CC[N+]1([O-])CCC[C@H]1CNC(=O)c1cc(S(=O)(=O)CC)c(N)cc1OC]
wherein the recited wt % is calculated relative to the total amount of (R)-(+)-amisulpride and (S)-(−)-amisulpride that is present in the pharmaceutical composition.

147. The pharmaceutical composition of any one of claims 135-146, comprising less than about 1.0 wt % of a compound of the following formula: 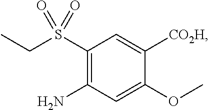 [SMILES: CCS(=O)(=O)c1cc(C(=O)O)c(OC)cc1N]
wherein the recited wt % is calculated relative to the total amount of (R)-(+)-amisulpride and (S)-(−)-amisulpride that is present in the pharmaceutical composition.

148. The pharmaceutical composition of any one of claims 135-147, comprising less than about 1.0 wt % of a compound of the following formula: 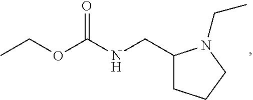
wherein the recited wt % is calculated relative to the total amount of (R)-(+)-amisulpride and (S)-(−)-amisulpride that is present in the pharmaceutical composition.

149. The pharmaceutical composition of any one of claims 135-148, comprising less than about 1.0 wt % of a compound of the following formula: 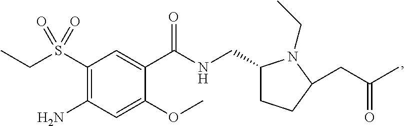 [SMILES: CCN1C(CC(C)=O)CC[C@@H]1CNC(=O)c1cc(S(=O)(=O)CC)c(N)cc1OC]
wherein the recited wt % is calculated relative to the total amount of (R)-(+)-amisulpride and (S)-(−)-amisulpride that is present in the pharmaceutical composition.

150. The pharmaceutical composition of any one of claims 135-149, comprising less than about 1.0 wt % of a compound of the following formula: 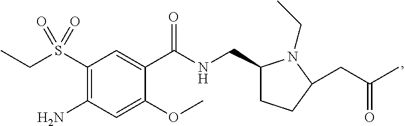 [SMILES: CCN1C(CC(C)=O)CC[C@H]1CNC(=O)c1cc(S(=O)(=O)CC)c(N)cc1OC]
wherein the recited wt % is calculated relative to the total amount of (R)-(+)-amisulpride and (S)-(−)-amisulpride that is present in the pharmaceutical composition.

151. The pharmaceutical composition of any one of claims 135-150, comprising less than about 1.0 wt % of a compound of the following formula: 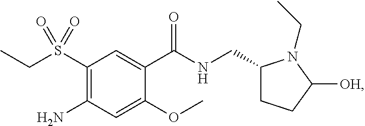 [SMILES: CCN1C(O)CC[C@@H]1CNC(=O)c1cc(S(=O)(=O)CC)c(N)cc1OC]
wherein the recited wt % is calculated relative to the total amount of (R)-(+)-amisulpride and (S)-(−)-amisulpride that is present in the pharmaceutical composition.

152. The pharmaceutical composition of any one of claims 135-151, comprising less than about 1.0 wt % of a compound of the following formula: 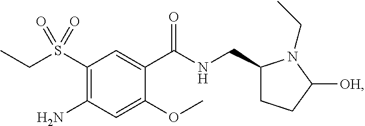 [SMILES: CCN1C(O)CC[C@H]1CNC(=O)c1cc(S(=O)(=O)CC)c(N)cc1OC]
wherein the recited wt % is calculated relative to the total amount of (R)-(+)-amisulpride and (S)-(−)-amisulpride that is present in the pharmaceutical composition.

153. A method of treating a psychiatric disorder in a subject comprising administering to the subject an enantiomerically pure crystalline form of (R)-(+)-amisulpride or (S)-(−)-amisulpride prepared by any one of claims 1-114, or an enantiomerically pure crystalline form of (R)-(+)-amisulpride or (S)-(−)-amisulpride of any one of claims 115-134, or a pharmaceutical composition of any one of claims 135-152.

154. The method according to claim 153, wherein the psychiatric disorder is a depressive order.

155. The method according to claim 153, wherein the psychiatric disorder is bipolar disorder.

156. The method according to claim 153, wherein the psychiatric disorder is bipolar depression.

157. The method according to claim 153, wherein the psychiatric disorder is major depressive disorder (MDD).

158. The method according to claim 153, wherein the psychiatric disorder is major depressive disorder with mixed features (MDD-MF).

159. The method according to claim 153, wherein the psychiatric disorder is treatment resistant depression (TRD).

160. The method according to claim 153, wherein the psychiatric disorder is schizophrenia.
